(19) United States
(12) Patent Application Publication
(10) Pub. No.: US 2011/0053907 A1
(43) Pub. Date: Mar. 3, 2011

(54) SUBSTITUTED PYRIMIDINES AND TRIAZINES AND THEIR USE IN CANCER THERAPY

(75) Inventors: Gordon William Rewcastle, Auckland (NZ); Peter Robin Shepherd, Auckland (NZ); Glaire Chaussade, Auckland (NZ); William Alexander Denny, Auckland (NZ); Swarnalatha Akuratiya Gamage, Auckland (NZ)

(73) Assignee: Auckland UniServices Limited, Auckland (NZ)

(21) Appl. No.: 12/934,616

(22) PCT Filed: Mar. 26, 2009
(86) PCT No.: PCT/NZ2009/000038

§ 371 Date: Nov. 10, 2010

Related U.S. Application Data
(60) Provisional application No. 61/040,064, filed on Mar. 27, 2008.

(51) Int. Cl. A61K 31/5377; C07D 413/14; C07D 401/14; C07D 487/04; A61K 31/53; A61P 35/00

(52) U.S. Cl. 514/210.18; 544/113; 544/216; 544/121; 514/210.2; 544/118; 514/236.2; 514/241; 514/235.8; 514/234.2

(57) ABSTRACT

Provided herein are substituted pyrimidine and triazine derivatives, including bicyclic pyrimidine derivatives, their pharmaceutical compositions, their preparation, and their use as agents or drugs for cancer therapy, either alone or in combination with radiation and/or other anticancer drugs. In one embodiment, the pyrimidine and triazine derivatives are morpholino-pyrimidine, morpholino-triazine, pyridyl-pyrimidine, and pyridyl-triazine derivatives which are selective irreversible inhibitors of the p110α isoform of PI3K.

### CROSS REFERENCE TO RELATED APPLICATION

This application claims the benefit of the priority of U.S. Provisional Application No. 61/040,064, filed Mar. 27, 2008, the disclosure of which is incorporated herein by reference in its entirety.

### FIELD

Provided herein are substituted pyrimidine and triazine derivatives, including bicyclic pyrimidine derivatives, their pharmaceutical compositions, their preparation, and their use as agents or drugs for cancer therapy, either alone or in combination with radiation and/or other anticancer drugs. In one embodiment, the substituted pyrimidine and triazine derivatives are morpholino-pyrimidine, morpholino-triazine, pyridyl-pyrimidine, and pyridyl-triazine derivatives which are selective irreversible inhibitors of the p110α isoform of PI3K.

### BACKGROUND

Phosphoinositide-3-kinases (PI3Ks) are a group of lipid kinases, which phosphorylate the 3-hydroxyl of phosphoinositides. They are classified into three classes (Classes I, II, and III) and play an important role in cellular signaling (Stephens et al., Curr. Opin. Pharmacol. 2005, 5, 357). The Class I enzymes are further classified into Class Ia and Ib based on their mechanism of activation; the Class Ia PI3Ks are heterodimeric structures consisting of a catalytic subunit (p110α, p110β, or p110δ) in complex with a regulatory p85 subunit, while the class-Ib PI3K (p110γ) is structurally similar but lacks the p85 regulatory subunit, and instead is activated by βγ subunits of heterotrimeric G-proteins (Walker et al., Mol. Cell. 2000, 6, 909). The human protein sequence of the p110α isoform is described in Volina et al., Genomics 1994, 24, 472; and Stirdivant et al., Bioorg. Med. Chem. 1997, 5, 65.

PI3Ks play a variety of roles in normal tissue physiology (Foukas & Shepherd, Biochem. Soc. Trans. 2004, 32, 330; Shepherd, Acta Physiol. Scand. 2005, 183, 3), with p110α having a specific role in cancer growth, p110β in thrombus formation mediated by integrin α_{II}β₃ (Jackson et al., Nat. Med. 2005, 11, 507), and p110γ in inflammation, rheumatoid arthritis (Camps et al., Nat. Med. 2005, 11, 936) and other chronic inflammation states (Barber et al., Nat. Med. 2005, 11, 933). The PI3K enzymes produce phosphoinositide 3,4,5-triphosphate (PIPS) from the corresponding diphosphate (PIP2), thus recruiting AKT (protein kinase B) through its Pleckstrin homology (PH) domain to the plasma membrane. Once bound, AKT is phosphorylated and activated by other membrane bound kinases and is central to a cascade of events that lead to inhibition of apoptosis (Berrie, Exp. Opin. Invest. Drugs 2001, 10, 1085).

The p110α isoform is selectively amplified and activated in a number of cancer types (Stephens et al., Curr. Opin. Pharmacol. 2005, 5, 357; Stauffer et al., Curr. Med. Chem.—Anti-Cancer Agents 2005, 5, 449). In addition, there is a high frequency of non-random mutations in specific sites, primarily in the C2 domain and or the activation loop, of the kinase in several human cancer cell lines, including colon, brain, breast, and stomach (Samuels et al., Science 2004, 304, 554). This results in a constitutively active enzyme (Ikenoue et al., Cancer Res. 2005, 65, 4562; Kang et al., Proc. Natl. Acad. Sci. USA 2005, 102, 802), making p110α one of the most highly mutated oncogenes found in human tumors. Structural studies have shown that many of the mutations occur at residues lying at the interfaces between p110α and p85α or between the kinase domain of p110α and other domains within the catalytic subunit (Miled et al., Science 2007, 317, 239; Huang et al., Science 2007, 318, 1744).

While PI3K isoenzymes play important roles in many cellular processes, published experimental studies in mice with human tumour xenografts show that the pan-PI3K inhibitor LY294002 is well-tolerated, reduces signalling through the PI3K pathway, causes reduction of tumour volume, and is more active in cell lines over-expressing mutant forms of p110α than parental control cells (Semba et al., Clin. Cancer Res. 2002, 8, 1957; Hu et al., Cancer Res. 2002, 62, 1087).

Thus, PI3K, especially the p110α isoform, is an interesting target for drug intervention. Several classes of compounds have been identified as reversible inhibitors; for example, LY 294002 (non-selective) (Walker et al., Mol. Cell. 2000, 6, 909), PI103 (slightly α-selective) (Knight et al., Cell 2006, 125, 733; Hayakawa et al., Bioorg. Med. Chem. Lett. 2007, 17, 2438; Raynaud et al., Cancer Res. 2007, 67, 5840), ZSTK474 (non-selective) (Yaguchi et al., J. Natl. Cancer Inst. 2006, 98, 545; Kong et al., Cancer Sci. 2007, 98, 1639), TGX221 (β-selective) (Jackson et al., Nat. Med. 2005, 11, 507), oxazines (γ-selective) (Lanni et al., Bioorg. Med. Chem. Lett. 2007, 17, 756), IC87114 (δ-selective) (Sadhu et al. WO 2001/81346; Billottet et al., Oncogene 2006, 25, 6648), AS605240 (γ-selective) (Camps et al., Nat. Med. 2005, 11, 936), the imidazo[1,2-a]pyridines (α-selective) (Hayakawa et al., Bioorg. Med. Chem. 2007, 15, 403; Hayakawa et al., Bioorg. Med. Chem. 2007, 15, 5837), and the imidazo[4,5-c]quinoline NVP-BEZ235 (Garcia-Echeverria, et al., WO 2006/122806).

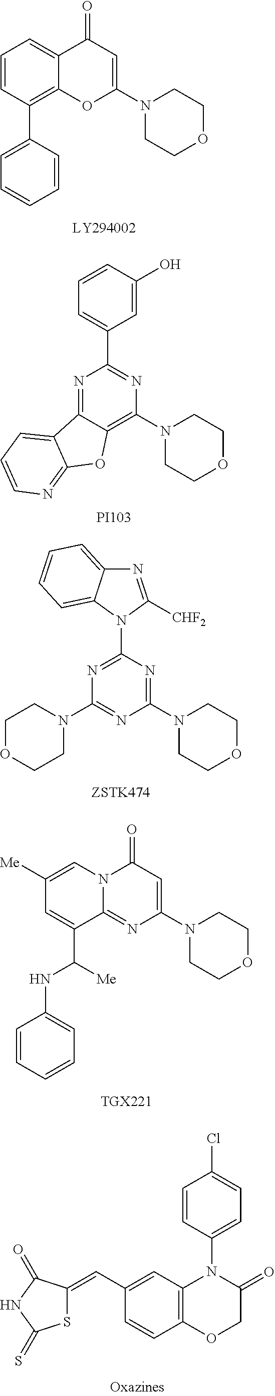

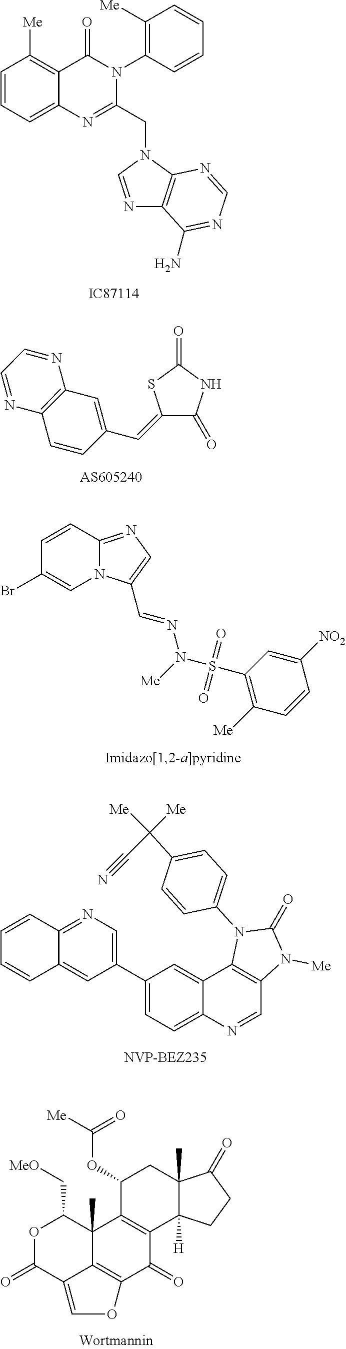

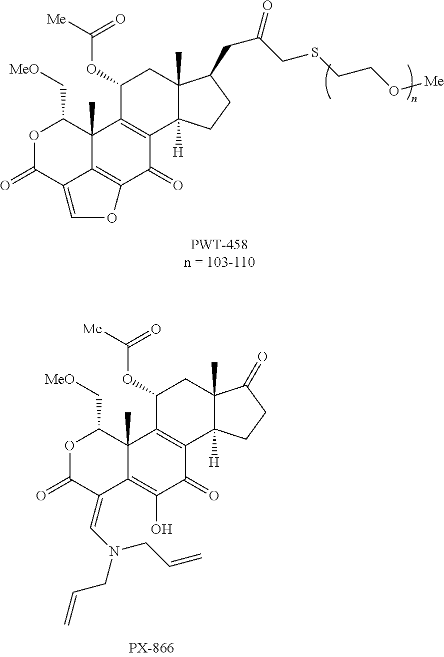

All of the above mentioned compounds function as reversible inhibitors of the appropriate PI3K isoforms. Although irreversible activity is displayed by the fungal metabolite wortmannin and its analogues, such as PWT-458 (Zhu et al, J. Med. Chem, 2006, 49, 1373) and PX-866 (Wipf et al., Org. Biomol. Chem. 2004, 2, 1911; Zask et al., J. Med. Chem. 2008, 51, 1319), these compounds are not selective for individual PI3K isoforms, undergoing reaction with a conserved lysine amino group (e.g., Lys-802 in p110α, Lys-805 in p110β, Lys-833 in p110γ, and Lys-799 in p110δ).

Despite the advances in developing PI3K inhibitors, there is an unmet need for PI3K inhibitors that are more potent and more selective, exhibit better pharmacokinetic properties, and/or produce fewer side effects than the existing PI3K inhibitors.

### SUMMARY OF THE DISCLOSURE

Provided herein is a compound of Formula Ia, Ib, Ic, or Id:

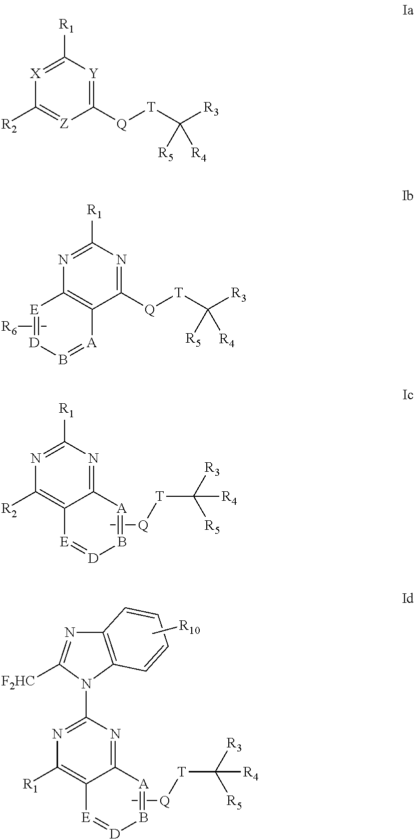

or an enantiomer, a mixture of enantiomers, or a mixture of two or more diastereomers thereof; or a pharmaceutically acceptable salt, solvate, hydrate, or prodrug thereof; wherein:

each R₁ is independently C₆₋₁₄ aryl, heteroaryl, or heterocyclyl;

each R₂ is independently C₆₋₁₄ aryl, heteroaryl, or heterocyclyl;

each R₃ and R₄ is independently hydrogen, lower alkyl, C₂₋₆ alkenyl, C₂₋₆ alkynyl, or R₅;

each R₅ is independently halogen or —OSO₂R₇;

R₆ is C₃₋₇ cycloalkyl, C₆₋₁₄ aryl, heteroaryl, or heterocyclyl;

R₇ is lower alkyl, C₂₋₆ alkenyl, C₂₋₆ alkynyl, C₃₋₇ cycloalkyl, C₆₋₁₄ aryl, heteroaryl, or heterocyclyl;

R₁₀ is (a) hydrogen, amino, or hydroxyl; or (b) lower alkyl, lower alkylamino, di(lower alkyl)amino, lower alkoxy, or carboxamido;

each Q is independently absent or a linker group;

each T is independently —CO—, —CS—, or —SO₂—;

X, Y, and Z are each independently a nitrogen atom or CR₈, with the proviso that at least two of X, Y, and Z are nitrogen atoms; wherein R₈ is hydrogen or lower alkyl; and

each A, B, D, and E is independently (i) a direct bond; (ii) a nitrogen, oxygen, or sulfur atom; or (iii) CR₉, where R₉ is hydrogen, halogen, or lower alkyl; wherein the bonds between A, B, D, and E may be saturated or unsaturated; with the proviso that no more than one of A, B, D, and E are a direct bond;

wherein each alkyl, alkenyl, alkynyl, alkoxy, alkylamino, dialkylamino, carboxamido, cycloalkyl, aryl, heteroaryl, and heterocyclyl is optionally substituted with one or more groups, each independently selected from (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl, each optionally substituted with one or more, in one embodiment, one, two, three, or four, substituents Q¹; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{x}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiment, one, two, three, or four, substituents Q¹; or (iii) R^{b} and R^{c} together with the N atom to which they are attached form heterocyclyl, optionally substituted with one or more, in one embodiment, one, two, three, or four, substituents Q¹;

wherein each Q¹ is independently selected from the group consisting of (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Also provided herein are pharmaceutical compositions comprising a compound disclosed herein, e.g., a compound of Formula I, including an enantiomer, a mixture of enantiomers, or a mixture of two or more diastereomers; or a pharmaceutically acceptable salt, solvate, hydrate, or prodrug thereof; in combination with one or more pharmaceutically acceptable carriers.

Additionally provided herein is a method for treating, preventing, or ameliorating one or more symptoms of a PI3K-mediated disorder, disease, or condition in a subject, comprising administering to the subject a therapeutically effective amount of a compound disclosed herein, e.g., a compound of Formula I, an enantiomer, a mixture of enantiomers, a mixture of two or more diastereomers; or a pharmaceutically acceptable salt, solvate, hydrate, or prodrug thereof.

Further provided herein is a method for modulating PI3K activity, comprising contacting a PI3K with a therapeutically effective amount of a compound disclosed herein, e.g., a compound of Formula I, including an enantiomer, a mixture of enantiomers, or a mixture of two or more diastereomers; or a pharmaceutically acceptable salt, solvate, hydrate, or prodrug thereof.

### BRIEF DESCRIPTION OF THE DRAWINGS

FIG. 1 shows the irreversible binding of the compound of Example 4 to PI3K.

### DETAILED DESCRIPTION

To facilitate understanding of the disclosure set forth herein, a number of terms are defined below.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The term “therapeutically effective amount” are meant to include the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins. Philadelphia, Pa., 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, 2nd Edition, Gibson Ed., CRC Press LLC: Boca Raton, Fla., 2009.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The term “alkyl” refers to a linear or branched saturated monovalent hydrocarbon radical, wherein the alkylene may optionally be substituted as described herein. The term “alkyl” also encompasses both linear and branched alkyl, unless otherwise specified. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, sec-butyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms.

The term “alkylene” refers to a linear or branched saturated divalent hydrocarbon radical, wherein the alkylene may optionally be substituted as described herein. The term “alkylene” encompasses both linear and branched alkylene, unless otherwise specified. In certain embodiments, the alkylene is a linear saturated divalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkylene groups are also referred as “lower alkylene.” Examples of alkylene groups include, but are not limited to, methylene, ethylene, propylene (including all isomeric forms), n-propylene, isopropylene, butylene (including all isomeric forms), n-butylene, isobutylene, t-butylene, pentylene (including all isomeric forms), and hexylene (including all isomeric forms). For example, C₁₋₆ alkylene refers to a linear saturated divalent hydrocarbon radical of 1 to 6 carbon atoms or a branched saturated divalent hydrocarbon radical of 3 to 6 carbon atoms.

The term “alkenyl” refers to a linear or branched monovalent hydrocarbon radical, which contains one or more, in one embodiment, one to five, carbon-carbon double bonds. The alkenyl may be optionally substituted as described herein. The term “alkenyl” also embraces radicals having “cis” and “trans” configurations, or alternatively, “Z” and “E” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propen-1-yl, propen-2-yl, allyl, butenyl, and 4-methylbutenyl.

The term “alkenylene” refers to a linear or branched divalent hydrocarbon radical, which contains one or more, in one embodiment, one to five, carbon-carbon double bonds. The alkenylene may be optionally substituted as described herein. Similarly, the term “alkenylene” also embraces radicals having “cis” and “trans” configurations, or alternatively, “E” and “Z” configurations. As used herein, the term “alkenylene” encompasses both linear and branched alkenylene, unless otherwise specified. For example, C₂₋₆ alkenylene refers to a linear unsaturated divalent hydrocarbon radical of 2 to 6 carbon atoms or a branched unsaturated divalent hydrocarbon radical of 3 to 6 carbon atoms. In certain embodiments, the alkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenylene groups include, but are not limited to, ethenylene, allylene, propenylene, butenylene, and 4-methylbutenylene.

The term “alkynyl” refers to a linear or branched monovalent hydrocarbon radical, which contains one or more, in one embodiment, one to five, carbon-carbon triple bonds. The alkynyl may be optionally substituted as described herein. The term “alkynyl” also encompasses both linear and branched alkynyl, unless otherwise specified. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkynyl groups include, but are not limited to, ethynyl (—C≡CH) and propargyl (—CH₂C≡CH). For example, C₂₋₆ alkynyl refers to a linear unsaturated monovalent hydrocarbon radical of 2 to 6 carbon atoms or a branched unsaturated monovalent hydrocarbon radical of 3 to 6 carbon atoms.

The term “alkynylene” refers to a linear or branched divalent hydrocarbon radical, which contains one or more, in one embodiment, one to five, carbon-carbon triple bonds. The alkynylene may be optionally substituted as described herein. The term “alkynylene” also encompasses both linear and branched alkynylene, unless otherwise specified. In certain embodiments, the alkynylene is a linear divalent hydrocarbon radical of 2 to 20 (C₂₋₂₀), 2 to 15 (C₂₋₁₅), 2 to 10 (C₂₋₁₀₎, or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkynylene groups include, but are not limited to, ethynylene (—C≡C—) and propargylene (—CH₂C≡C—). For example, C₂₋₆ alkynylene refers to a linear unsaturated divalent hydrocarbon radical of 2 to 6 carbon atoms or a branched unsaturated divalent hydrocarbon radical of 3 to 6 carbon atoms.

The term “cycloalkyl” refers to a cyclic saturated bridged and/or non-bridged monovalent hydrocarbon radical, which may be optionally substituted as described herein. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclohexyl, cycloheptyl, bicyclo[2.1.1]hexyl, bicyclo[2.2.1]heptyl, decalinyl, and adamantyl.

The term “cycloalkylene” refers to a cyclic saturated bridged and/or non-bridged divalent hydrocarbon radical, which may be optionally substituted as described herein. In certain embodiments, the cycloalkylene has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkylene groups include, but are not limited to, cyclopropylene (e.g., 1,1-cyclopropylene and 1,2-cyclopropylene), cyclobutylene (e.g., 1,1-cyclobutylene, 1,2-cyclobutylene, or 1,3-cyclobutylene), cyclopentylene (e.g., 1,1-cyclopentylene, 1,2-cyclopentylene, or 1,3-cyclopentylene), cyclohexylene (e.g., 1,1-cyclohexylene, 1,2-cyclohexylene, 1,3-cyclohexylene, or 1,4-cyclohexylene), cycloheptylene (e.g., 1,1-cycloheptylene, 1,2-cycloheptylene, 1,3-cycloheptylene, or 1,4-cycloheptylene), decalinylene, and adamantylene.

The term “aryl” refers to a monocyclic aromatic group and/or multicyclic monovalent aromatic group that contain at least one aromatic hydrocarbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In certain embodiments, aryl may be optionally substituted as described herein.

The term “arylene” refers to a monocyclic and/or multicyclic divalent aromatic group that contain at least one aromatic hydrocarbon ring. In certain embodiments, the arylene has from 6 to 20 (C₆₋₂₀), from 6 to 15 (C₆₋₁₅), or from 6 to 10 (C₆₋₁₀) ring atoms. Examples of arylene groups include, but are not limited to, phenylene, naphthylene, fluorenylene, azulenylene, anthrylene, phenanthrylene, pyrenylene, biphenylene, and terphenylene. Arylene also refers to bicyclic or tricyclic carbon rings, where one of the rings is aromatic and the others of which may be saturated, partially unsaturated, or aromatic, for example, dihydronaphthylene, indenylene, indanylene, or tetrahydro-naphthylene (tetralinyl). In certain embodiments, arylene may also be optionally substituted as described herein.

The term “aralkyl” or “aryl-alkyl” refers to a monovalent alkyl group substituted with aryl. In certain embodiments, the alkyl and aryl moieties are optionally substituted as described herein.

The term “heteroaryl” refers to a monocyclic aromatic group and/or multicyclic aromatic group that contain at least one aromatic ring, wherein at least one aromatic ring contains one or more heteroatoms independently selected from O, S, and N. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzothiophenyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted as described herein.

The term “heteroarylene” refers to a divalent aromatic group and/or multicyclic aromatic group that contain at least one aromatic ring, wherein at least one aromatic ring contains one or more heteroatoms independently selected from O, S, and N. Each ring of a heteroarylene group can contain one or two O atoms, one or two S atoms, and/or one to four N atoms, provided that the total number of heteroatoms in each ring is four or less and each ring contains at least one carbon atom. In certain embodiments, the heteroarylene has from 5 to 20, from 5 to 15, or from 5 to 10 ring atoms. Examples of monocyclic heteroarylene groups include, but are not limited to, furanylene, imidazolylene, isothiazolylene, isoxazolylene, oxadiazolylene, oxadiazolylene, oxazolylene, pyrazinylene, pyrazolylene, pyridazinylene, pyridylene, pyrimidinylene, pyrrolylene, thiadiazolylene, thiazolylene, thienylene, tetrazolylene, triazinylene, and triazolylene. Examples of bicyclic heteroarylene groups include, but are not limited to, benzofuranylene, benzimidazolylene, benzoisoxazolylene, benzopyranylene, benzothiadiazolylene, benzothiazolylene, benzothienylene, benzothiophenylene, benzotriazolylene, benzoxazolylene, furopyridylene, imidazopyridinylene, imidazothiazolylene, indolizinylene, indolylene, indazolylene, isobenzofuranylene, isobenzothienylene, isoindolylene, isoquinolinylene, isothiazolylene, naphthyridinylene, oxazolopyridinylene, phthalazinylene, pteridinylene, purinylene, pyridopyridylene, pyrrolopyridylene, quinolinylene, quinoxalinylene, quinazolinylene, thiadiazolopyrimidylene, and thienopyridylene. Examples of tricyclic heteroaryl groups include, but are not limited to, acridinylene, benzindolylene, carbazolylene, dibenzofuranylene, perimidinylene, phenanthrolinylene, phenanthridinylene, phenarsazinylene, phenazinylene, phenothiazinylene, phenoxazinylene, and xanthenylene. In certain embodiments, heteroaryl may also be optionally substituted as described herein.

The term “heterocyclyl” or “heterocyclic” refers to a monocyclic non-aromatic ring system and/or multicyclic ring system that contains at least one non-aromatic ring, wherein one or more of the non-aromatic ring atoms are heteroatoms independently selected from O, S, or N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may include a fused or bridged ring system, and in which the nitrogen or sulfur atoms may be optionally oxidized, the nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic radicals include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, β-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, heterocyclic may also be optionally substituted as described herein.

The term “heterocyclylene” refers to a divalent non-aromatic ring system and/or multicyclic ring system that contain at least one non-aromatic ring, wherein one or more of the non-aromatic ring atoms are heteroatoms independently selected from O, S, or N; and the remaining ring atoms are carbon atoms. In certain embodiments, the heterocyclylene group has from 3 to 20, from 3 to 15, from 3 to 10, from 3 to 8, from 4 to 7, or from 5 to 6 ring atoms. In certain embodiments, the heterocyclylene is a monocyclic, bicyclic, tricyclic, or tetracyclic ring system, which may include a fused or bridged ring system, and in which the nitrogen or sulfur atoms may be optionally oxidized, the nitrogen atoms may be optionally quaternized, and some rings may be partially or fully saturated, or aromatic. The heterocyclylene may be attached to the main structure at any heteroatom or carbon atom which results in the creation of a stable compound. Examples of such heterocyclene groups include, but are not limited to, azepinylene, benzodioxanylene, benzodioxolylene, benzofuranonylene, benzopyranonylene, benzopyranylene, benzotetrahydrofuranylene, benzotetrahydrothienylene, benzothiopyranylene, benzoxazinylene, β-carbolinylene, chromanylene, chromonylene, cinnolinylene, coumarinylene, decahydroisoquinolinylene, dihydrobenzisothiazinylene, dihydrobenzisoxazinylene, dihydrofurylene, dihydroisoindolylene, dihydropyranylene, dihydropyrazolylene, dihydropyrazinylene, dihydropyridinylene, dihydropyrimidinylene, dihydropyrrolylene, dioxolanylene, 1,4-dithianylene, furanonylene, imidazolidinylene, imidazolinylene, indolinylene, isobenzotetrahydrofuranylene, isobenzotetrahydrothienylene, isochromanylene, isocoumarinylene, isoindolinylene, isothiazolidinylene, isoxazolidinylene, morpholinylene, octahydroindolylene, octahydroisoindolylene, oxazolidinonylene, oxazolidinylene, oxiranylene, piperazinylene, piperidinylene, 4-piperidonylene, pyrazolidinylene, pyrazolinylene, pyrrolidinylene, pyrrolinylene, quinuclidinylene, tetrahydrofurylene, tetrahydroisoquinolinylene, tetrahydropyranylene, tetrahydrothienylene, thiamorpholinylene, thiazolidinylene, tetrahydroquinolinylene, and 1,3,5-trithianylene. In certain embodiments, heterocyclic may also be optionally substituted as described herein.

The term “halogen”, “halide” or “halo” refers to fluorine, chlorine, bromine, and/or iodine.

The term “optionally substituted” is intended to mean that a group, such as an alkyl, alkylene, alkenyl, alkenylene, alkynyl, alkynylene, alkoxy, alkylamino, dialkylamino, carboxamido, cycloalkyl, cycloalkylene, aryl, arylene, heteroaryl, heteroarylene, heterocyclyl, or heterocyclylene, may be substituted with one or more substituents independently selected from, e.g., (a) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl, each optionally substituted with one or more, in one embodiment, one, two, three, or four, substituents Q¹; and (b) halo, cyano (—CN), nitro (—NO₂), —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiment, one, two, three, or four, substituents Q¹; or (iii) R^{b} and R^{c} together with the N atom to which they are attached form heteroaryl or heterocyclyl, optionally substituted with one or more, in one embodiment, one, two, three, or four, substituents Q¹. As used herein, all groups that can be substituted are “optionally substituted,” unless otherwise specified.

In one embodiment, each Q¹ is independently selected from the group consisting of (a) cyano, halo, and nitro; and (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{f} and R^{g} together with the N atom to which they are attached form heteroaryl or heterocyclyl.

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of the desired enantiomer and about 5% or less of the less preferred enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

The term “solvate” refers to a compound provided herein or a salt thereof, which further includes a stoichiometric or non-stoichiometric amount of solvent bound by non-covalent intermolecular forces. Where the solvent is water, the solvate is a hydrate.

### Compounds

Compounds that have been shown to have activity against PI3K have been disclosed in WO 2004/048365 (Chiron), WO 2007/066099 and 2007/066103 (AstraZeneca), WO 2007/084786 (Novartis), WO 2006/095906 (Zenyaku Kogyo Kabushiki Kaisha), WO 2007/127183 (Genentech/Piramed), WO 2008/018426 (Chugai Seiyaku Kabushiki Kaisha), and WO2008/0322028, WO2008/0322033, WO2008/0322036, WO2008/0322041, WO2008/0322060, WO2008/0322064, WO2008/0322072, WO2008/0322089, WO2008/0322091 (AstraZeneca), WO2008/115974, and WO2008/116129 (Wyeth).

The present disclosure broadly relates to a class of compounds for use as agents or drugs for cancer therapy. In certain embodiments, the disclosure relates to a class of compounds that can be used as PI3K inhibitors. PI3K inhibitors are thought to be valuable for the treatment of cell proliferation disorders, in one embodiment, as anti tumor agents.

In one embodiment, provided herein is a compound of Formula Ia, Ib, Ic, or Id:

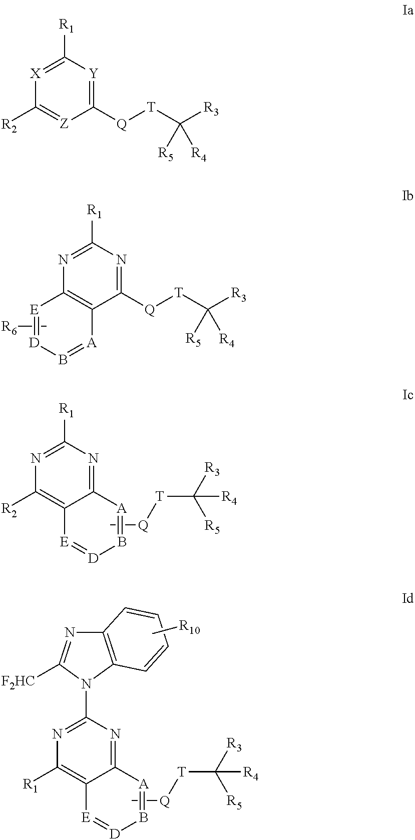

or an enantiomer, a mixture of enantiomers, or a mixture of two or more diastereomers thereof; or a pharmaceutically acceptable salt, solvate, hydrate, or prodrug thereof; wherein:

each R₁ is independently C₆₋₁₄ aryl, heteroaryl, or heterocyclyl;

each R₂ is independently C₆₋₁₄ aryl, heteroaryl, or heterocyclyl;

each R₃ and R₄ is independently hydrogen, lower alkyl, C₂₋₆ alkenyl, C₂₋₆ alkynyl, or R₅;

each R₅ is independently halogen or —OSO₂R₇;

R₆ is C₃₋₇ cycloalkyl, C₆₋₁₄ aryl, heteroaryl, or heterocyclyl;

R₇ is lower alkyl, C₂₋₆ alkenyl, C₂₋₆ alkynyl, C₃₋₇ cycloalkyl, C₆₋₁₄ aryl, heteroaryl, or heterocyclyl;

R₁₀ is (a) hydrogen, amino, or hydroxyl; or (b) lower alkyl, lower alkylamino, di(lower alkyl)amino, lower alkoxy, or carboxamido;

each Q is independently absent or a linker group;

each T is independently —CO—, —CS—, or —SO₂—;

X, Y, and Z are each independently a nitrogen atom or CR₈, with the proviso that at least two of X, Y, and Z are nitrogen atoms; wherein R₈ is hydrogen or lower alkyl; and

each A, B, D, and E is independently (i) a direct bond; (ii) a nitrogen, oxygen, or sulfur atom; or (iii) CR₉, where R₉ is hydrogen, halogen, or lower alkyl; wherein the bonds between A, B, D, and E may be saturated or unsaturated; with the proviso that no more than one of A, B, D, and E are a direct bond;

wherein each alkyl, alkenyl, alkynyl, alkoxy, alkylamino, dialkylamino, carboxamido, cycloalkyl, aryl, heteroaryl, and heterocyclyl is optionally substituted with one or more groups, each independently selected from (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl, each optionally substituted with one or more, in one embodiment, one, two, three, or four, substituents Q¹; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiment, one, two, three, or four, substituents Q¹; or (iii) R^{b} and R^{c} together with the N atom to which they are attached form heterocyclyl, optionally substituted with one or more, in one embodiment, one, two, three, or four, substituents Q¹;

wherein each Q¹ is independently selected from the group consisting of (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In one embodiment, in Formula Ia, Ib, Ic, or Id:

each R₁ is independently substituted or unsubstituted heteroaryl, or heterocyclyl;

each R₂ is independently substituted or unsubstituted C₆₋₁₄ aryl, heteroaryl, or heterocyclyl;

each R₃ and R₄ is independently hydrogen, lower alkyl, or R₅;

each R₅ is independently halogen or —OSO₂R₇;

R₆ is substituted or unsubstituted C₆₋₁₄ aryl or heteroaryl;

R₇ is substituted or unsubstituted lower alkyl or C₆₋₁₄ aryl;

R₁₀ is (a) hydrogen, amino, or hydroxyl; or (b) substituted or unsubstituted lower alkyl, lower alkylamino, di(lower alkyl)amino, lower alkoxy, or carboxamido;

each Q is independently absent or a linker group;

each T is independently —CO—, —CS—, or —SO₂—;

X, Y, and Z are each independently a nitrogen atom or CR₈, with the proviso that at least two of X, Y, and Z are nitrogen atoms; wherein R₈ is hydrogen or lower alkyl; and

each A, B, D, and E is independently (i) a direct bond; (ii) a nitrogen, oxygen, or sulfur atom; or (iii) CR₉, where R₉ is hydrogen, lower alkyl, or halogen; wherein the bonds between A, B, D, and E may be saturated or unsaturated.

In another embodiment, in Formula Ia, Ib, Ic, or Id:

each R₁ is independently substituted or unsubstituted morpholino or pyridyl;

each R₂ is independently substituted or unsubstituted aryl, heteroaryl, or heterocyclyl;

each R₃ and R₄ is independently hydrogen, lower alkyl, or R₅;

each R₅ is independently halogen or —OSO₂R₇;

R₆ is substituted or unsubstituted aryl or heteroaryl;

R₇ is substituted or unsubstituted lower alkyl or aryl;

R₁₀ is (a) hydrogen, amino, hydroxyl, or hydroxymethyl; or (b) substituted or unsubstituted lower alkylamino, di(lower alkyl)amino, lower alkoxy, or carboxamido;

each Q is independently absent or a linker group;

each T is independently —CO—, —CS—, or —SO₂—;

X, Y, and Z are each independently a nitrogen atom or CR₈, with the proviso that at least two of X, Y, and Z are nitrogen atoms; where R₈ is hydrogen or lower alkyl; and

each A, B, D, and E is independently (i) a direct bond; (ii) a nitrogen, oxygen, or sulfur atom; or (iii) CR₉, where R₉ is hydrogen, lower alkyl, or halogen; wherein the bonds between A, B, D, and E may be saturated or unsaturated.

In still another embodiment, in Formula Ia, Ib, Ic, or Id:

each R₁ is independently substituted or unsubstituted 4-morpholino or 4-pyridyl;

each R₂ is independently substituted or unsubstituted aryl, heteroaryl, or heterocyclyl;

each R₃ and R₄ is independently hydrogen, lower alkyl, or R₅;

each R₅ is independently halogen or —OSO₂R₇;

R₆ is substituted or unsubstituted aryl or heteroaryl;

R₇ is substituted or unsubstituted lower alkyl or aryl;

R₁₀ is hydrogen, amino, lower alkylamino, substituted lower alkylamino, hydroxy, lower alkoxy, substituted lower alkoxy, hydroxymethyl, carboxamido, or substituted carboxamido;

each Q is independently absent or a linker group;

each T is independently —CO—, —CS—, or —SO₂—;

X, Y, and Z are each independently a nitrogen atom or CR₈, with the proviso that at least two of X, Y, and Z are nitrogen atoms; where R₈ is hydrogen or lower alkyl; and

each A, B, D, and E is independently (i) a direct bond; (ii) a nitrogen, oxygen, or sulfur atom; or (iii) CR₉, where R₉ is hydrogen, lower alkyl, or halogen; wherein the bonds between A, B, D, and E may be saturated or unsaturated.

In one embodiment, in Formula Ia:

R₁ is substituted or unsubstituted morpholino or pyridyl;

R₂ is hydroxyphenyl, hydroxymethylphenyl, aminopyridyl, aminopyrimidyl, indazolyl, difluoromethyl-1H-benzimidazolyl, difluoromethyl-hydroxy-1H-benzimidazolyl, difluoromethyl-methoxy-1H-benzimidazolyl, difluoromethyl-ethoxy-1H-benzimidazolyl, or difluoromethyl-((N,N-dimethylamino)propoxy)-1H-benzimidazolyl;

R₃ and R₄ are each independently hydrogen, chloro, or methyl;

R₅ is chloro, bromo, or —OSO₂-methyl;

Q is a divalent linker selected from the group consisting of azetidinyleneamino, azetidinylene(methylamino), piperidyleneoxy, piperazinylene, piperidylene, piperidyleneamino, piperidylene(methylamino), pyrrolidinyleneamino, pyrrolidinylene(methylamino), and piperidylenethio;

T is —CO—, —CS—, or —SO₂—; and

X, Y, and Z are each independently a nitrogen atom or CH, with the proviso that at least two of X, Y, and Z are nitrogen atoms.

In another embodiment, in Formula Ia:

R₁ is substituted or unsubstituted 4-morpholino or 4-pyridyl;

R₂ is 3-hydroxyphenyl, 3-hydroxymethylphenyl, 2-amino-pyrid-5-yl, 2-amino-pyrimid-5-yl, indazol-4-yl, 2-difluoromethyl-1H-benzimidazolyl, 2-difluoromethyl-4-hydroxy-1H-benzimidazolyl, 2-difluoromethyl-4-methoxy-1H-benzimidazolyl, 2-difluoromethyl-4-ethoxy-1H-benzimidazolyl, or 2-difluoromethyl-4-(3-(N,N-dimethylamino)propoxy)-1H-benzimidazolyl;

R₃ and R₄ are each independently hydrogen, chloro, or methyl;

R₅ is chloro, bromo, or —OSO₂-methyl;

Q is a divalent linker selected from the group consisting of azetidinylene-4-amino, azetidinylene-4-methylamino, piperidylene-4-oxy, 1,4-piperazinylene, 1,4-piperidylene, piperidylene-3-amino, (R)-piperidylene-3-amino, (S)-piperidylene-3-amino, piperidylene-3-methylamino, (R)-piperidylene-3-methylamino, (S)-piperidylene-3-methylamino, piperidylene-4-amino, piperidylene-4-methylamino, pyrrolidinylene-3-amino, (R)-pyrrolidinylene-3-amino, (S)-pyrrolidinylene-3-amino, pyrrolidinylene-3-methylamino, (R)-pyrrolidinylene-3-methylamino, (S)-pyrrolidinylene-3-methylamino, and piperidylene-4-thio;

T is —CO—, —CS—, or —SO₂—; and

X, Y, and Z are each independently a nitrogen atom or CH, with the proviso that at least two of X, Y, and Z are nitrogen atoms.

Examples of the linker group defined by Q may include, but are not restricted to, alkylene, alkenylene, alkynylene, cycloalkylene, cycloalkenylene, arylene, heteroarylene, heterocyclylene, tetrahydropyridinylene, divalent amino (—NH—), alkyleneamino, substituted alkyleneamino, alkenyleneamino, cycloalkyleneamino, cycloalkenyleneamino, aryleneamino, heteroaryleneamino, heterocyclyleneamino, divalent aminoarylamino, divalent aminoheteroarylamino, divalent aminoheterocyclylamino, tetrahydropyridinyleneamino, azetidinylene, pyrrolidinylene, piperidinylene, piperazinylene, azetidinyleneamino, pyrrolidinyleneamino, piperidinyleneamino, piperazinyleneamino, azetidinylenecarbonylamino, pyrrolidinylenecarbonylamino, piperidinylenecarbonylamino, piperazinylenecarbonylamino, alkyleneoxy, alkenyleneoxy, alkynyleneoxy, cycloalkyleneoxy, cycloalkenyleneoxy, aryleneoxy, heteroaryleneoxy, heterocyclyleneoxy, divalent aminoalkoxy, divalent aminoalkenyloxy, divalent aminoalkynyloxy, divalent aminocycloalkyloxy, divalent aminocycloalkenyloxy, divalent aminoaryloxy, divalent aminoheteroaryloxy, azetidinyleneoxy, pyrrolidinyleneoxy, piperidinyleneoxy, piperazinyleneoxy, alkylenethio, alkenylenethio, alkynylenethio, cycloalkylenethio, cycloalkenylenethio, arylenethio, heteroarylenethio, heterocyclylenethio, divalent aminoalkylthio, divalent aminoalkenylthio, divalent aminoalkynylthio, divalent aminocycloalkylthio, divalent aminocycloalkenylthio, divalent aminoarylthio, divalent aminoheteroarylthio, and divalent aminoheterocyclylthio.

Examples of R₂ may include, but are not restricted to, 3-hydroxyphenyl, 3-(hydroxymethyl)phenyl, 3-pyridinyl, 4-pyridinyl, 3-pyridinylamino, 4-pyridinylamino, 4-indazolyl, 4-indazolylamino, 5-indazolylamino, 6-indazolylamino 7-indazolylamino, 1-benzimidazolyl, 2-methyl-1-benzimidazolyl, 2-fluoromethyl-1-benzimidazolyl, 2-difluoromethyl-1-benzimidazolyl, 2-trifluoromethyl-1-benzimidazolyl, 2-difluoromethyl-4-hydroxy-1-benzimidazolyl, 4-alkoxy-2-difluoromethyl-1-benzimidazolyl, 4-amino-2-difluoromethyl-1-benzimidazolyl, 4-alkylamino-2-difluoromethyl-1-benzimidazolyl, 2-amino-5-pyridinyl, 2-amino-4-chloro-5-pyridinyl, 2-amino-4-methyl-5-pyridinyl, 2-amino-4-trifluoromethyl-5-pyridinyl, 2-amino-4-cyano-5-pyridinyl, 2-amino-4-methoxy-5-pyridinyl, 2-amino-5-pyrimidinyl, 2,4-diamino-5-pyrimidinyl, 2-amino-4-methyl-5-pyrimidinyl, 2-amino-4-trifluoromethyl-5-pyrimidinyl, 2-amino-4-chloro-5-pyrimidinyl, 2-amino-4-cyano-5-pyrimidinyl, 2-amino-4-methoxy-5-pyrimidinyl, and 2-amino-4-oxo-5-pyrimidinyl.

Examples of R₆ may include, but are not restricted to, 3-hydroxyphenyl, 3-(hydroxymethyl)phenyl, 3-pyridinyl, 4-pyridinyl, 4-indazolyl, 5-indazolyl, 6-indazolyl, 7-indazolyl, 2-amino-5-pyridinyl, 2-amino-4-chloro-5-pyridinyl, 2-amino-4-methyl-5-pyridinyl, 2-amino-4-trifluoromethyl-5-pyridinyl, 2-amino-4-cyano-5-pyridinyl, 2-amino-4-methoxy-5-pyridinyl, 2-amino-5-pyrimidinyl, 2,4-diamino-5-pyrimidinyl, 2-amino-4-methyl-5-pyrimidinyl, 2-amino-4-trifluoromethyl-5-pyrimidinyl, 2-amino-4-chloro-5-pyrimidinyl, 2-amino-4-cyano-5-pyrimidinyl, 2-amino-4-methoxy-5-pyrimidinyl, 2-amino-4-oxo-5-pyrimidinyl.

In one embodiment, the compound of Formula Ia has the structure of Formula II:

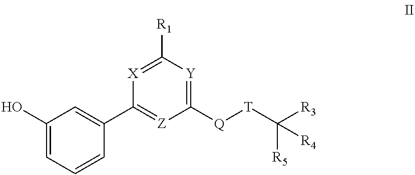

or an enantiomer, a mixture of enantiomers, or a mixture of two or more diastereomers thereof; or a pharmaceutically acceptable salt, solvate, hydrate, or prodrug thereof; wherein R₁, R₃, R₄, R₅, Q, T, X, Y, and Z are each as defined herein.

In one embodiment, in Formula II,

R₁ is substituted or unsubstituted 4-morpholino or 4-pyridyl;

R₃ and R₄ are hydrogen, lower alkyl, substituted lower alkyl, or R₅;

R₅ is halogen or —OSO₂R₇;

R₇ is substituted or unsubstituted lower alkyl or aryl;

Q is a linker group as defined herein;

T is —CO—, —CS—, or —SO₂—; and

X, Y, and Z are nitrogen atoms or CR₈, except that at least two of X, Y, and Z are nitrogen atoms; where R₈ is hydrogen or lower alkyl.

In another embodiment, the compound of Formula Ia has the structure of Formula III:

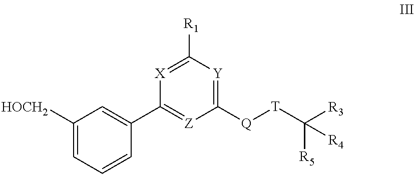

or an enantiomer, a mixture of enantiomers, or a mixture of two or more diastereomers thereof; or a pharmaceutically acceptable salt, solvate, hydrate, or prodrug thereof; wherein R₁, R₃, R₄, R₅, Q, T, X, Y, and Z are each as defined herein.

In one embodiment, in Formula III,

R₁ is substituted or unsubstituted 4-morpholino or 4-pyridyl;

R₃ and R₄ are hydrogen, lower alkyl, substituted lower alkyl, or R₅;

R₅ is halogen or —OSO₂R₇;

R₇ is substituted or unsubstituted lower alkyl or aryl;

Q is a linker group as defined herein;

T is —CO—, —CS—, or —SO₂—; and

X, Y, and Z are nitrogen atoms or CR₈, except that at least two of X, Y, and Z are nitrogen atoms; where R₈ is hydrogen or lower alkyl.

In yet another embodiment, the compound of Formula Ia has the structure of Formula IV:

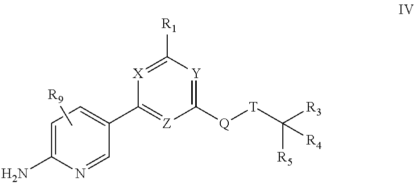

or an enantiomer, a mixture of enantiomers, or a mixture of two or more diastereomers thereof; or a pharmaceutically acceptable salt, solvate, hydrate, or prodrug thereof; wherein:

R₁, R₃, R₄, R₅, Q, T, X, Y, and Z are each as defined herein; and

R₉ is hydrogen, lower alkyl, fluoromethyl, difluoromethyl, trifluoromethyl, cyano, amino, lower alkylamino, hydroxyl, lower alkoxy, or halogen.

In one embodiment, in Formula IV,

R₁ is substituted or unsubstituted 4-morpholino or 4-pyridyl;

R₃ and R₄ are hydrogen, lower alkyl, substituted lower alkyl, or R₅;

R₅ is halogen or —OSO₂R₇;

R₇ is substituted or unsubstituted lower alkyl or aryl;

Q is a linker group as defined herein;

T is —CO—, —CS—, or —SO₂—;

X, Y, and Z are nitrogen atoms or CR₈, except that at least two of X, Y, and Z are nitrogen atoms; where R₈ is hydrogen or lower alkyl; and

R₉ is hydrogen, lower alkyl, fluoromethyl, difluoromethyl, trifluoromethyl, cyano, amino, lower alkylamino, hydroxyl, lower alkoxy, or halogen.

In yet another embodiment, the compound of Formula Ia has the structure of Formula V:

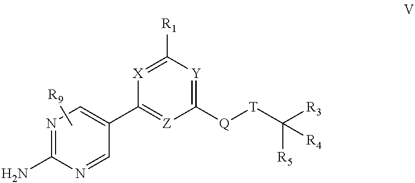

or an enantiomer, a mixture of enantiomers, or a mixture of two or more diastereomers thereof; or a pharmaceutically acceptable salt, solvate, hydrate, or prodrug thereof; wherein:

R₁, R₃, R₄, R₅, Q, T, X, Y, and Z are each as defined herein; and

R₉ is hydrogen, lower alkyl, fluoromethyl, difluoromethyl, trifluoromethyl, cyano, amino, lower alkylamino, hydroxyl, lower alkoxy, or halogen.

In one embodiment, in Formula V,

R₁ is substituted or unsubstituted 4-morpholino or 4-pyridyl;

R₃ and R₄ are hydrogen, lower alkyl, substituted lower alkyl, or R₅;

R₅ is halogen or —OSO₂R₇;

R₇ is substituted or unsubstituted lower alkyl or aryl;

Q is a linker group as defined herein;

T is —CO—, —CS—, or —SO₂—;

X, Y, and Z are nitrogen atoms or CR₈, except that at least two of X, Y, and Z are nitrogen atoms; where R₈ is hydrogen or lower alkyl; and

R₉ is hydrogen, lower alkyl, fluoromethyl, difluoromethyl, trifluoromethyl, cyano, amino, lower alkylamino, hydroxyl, lower alkoxy, or halogen.

In yet another embodiment, the compound of Formula Ia has the structure of Formula VI:

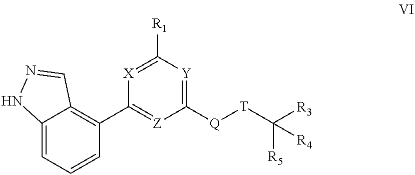

or an enantiomer, a mixture of enantiomers, or a mixture of two or more diastereomers thereof; or a pharmaceutically acceptable salt, solvate, hydrate, or prodrug thereof; wherein R₁, R₃, R₄, R₅, Q, T, X, Y, and Z are each as defined herein.

In one embodiment, in Formula VI,

R₁ is substituted or unsubstituted 4-morpholino or 4-pyridyl;

R₃ and R₄ are hydrogen, lower alkyl, substituted lower alkyl, or R₅;

R₅ is halogen or —OSO₂R₇;

R₇ is substituted or unsubstituted lower alkyl or aryl;

Q is a linker group as defined herein;

T is —CO—, —CS—, or —SO₂—; and

X, Y, and Z are nitrogen atoms or CR₈, except that at least two of X, Y, and Z are nitrogen atoms; where R₈ is hydrogen or lower alkyl.

In yet another embodiment, the compound of Formula Ia has the structure of Formula VII:

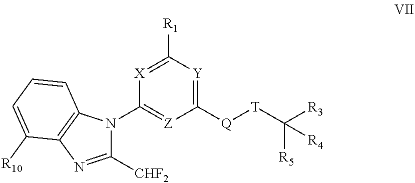

or an enantiomer, a mixture of enantiomers, or a mixture of two or more diastereomers thereof; or a pharmaceutically acceptable salt, solvate, hydrate, or prodrug thereof; wherein R₁, R₃, R₄, R₅, R₁₀, Q, T, X, Y, and Z are each as defined herein.

In one embodiment, in Formula VII,

R₁ is substituted or unsubstituted 4-morpholino or 4-pyridyl;

R₃ and R₄ are hydrogen, lower alkyl, substituted lower alkyl, or R₅;

R₅ is halogen or —OSO₂R₇;

R₇ is substituted or unsubstituted lower alkyl or aryl;

Q is a linker group as defined herein;

T is —CO—, —CS—, or —SO₂—;

X, Y, and Z are nitrogen atoms or CR₈, except that at least two of X, Y, and Z are nitrogen atoms; where R₈ is hydrogen or lower alkyl; and

R₁₀ is hydrogen, amino, lower alkylamino, substituted lower alkylamino, hydroxy, lower alkoxy, substituted lower alkoxy, hydroxymethyl, carboxamido, or substituted carboxamido.

In yet another embodiment, the compound of Formula Ia has the structure of Formula VIII:

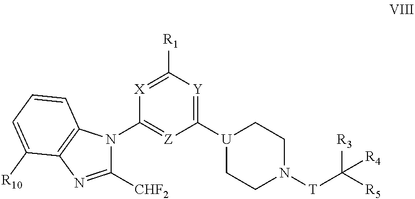

or an enantiomer, a mixture of enantiomers, or a mixture of two or more diastereomers thereof; or a pharmaceutically acceptable salt, solvate, hydrate, or prodrug thereof; wherein:

R₁, R₃, R₄, R₅, R₁₀, T, X, Y, and Z are each as defined herein; and

U is CH or N.

In one embodiment, in Formula VIII,

R₁ is substituted or unsubstituted 4-morpholino or 4-pyridyl;

R₃ and R₄ are hydrogen, lower alkyl, substituted lower alkyl, or R₅;

R₅ is halogen or —OSO₂R₇;

R₇ is substituted or unsubstituted lower alkyl or aryl;

T is —CO—, —CS—, or —SO₂—;

X, Y, and Z are nitrogen atoms or CR₈, except that at least two of X, Y, and Z are nitrogen atoms; where R₈ is hydrogen or lower alkyl;

U is CH or N; and

R₁₀ is hydrogen, amino, lower alkylamino, substituted lower alkylamino, hydroxy, lower alkoxy, substituted lower alkoxy, hydroxymethyl, carboxamido, or substituted carboxamido.

In yet another embodiment, the compound of Formula Ia has the structure of Formula IX:

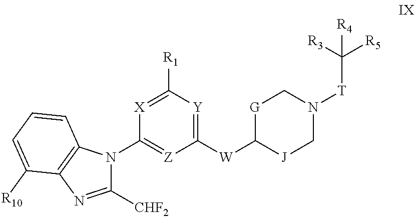

or an enantiomer, a mixture of enantiomers, or a mixture of two or more diastereomers thereof; or a pharmaceutically acceptable salt, solvate, hydrate, or prodrug thereof; wherein:

R₁, R₃, R₄, R₅, R₁₀, T, X, Y, and Z are each as defined herein;

G and J are each independently a direct bond or —CH₂—; and

W is a direct bond; or oxygen, sulfur, or NR₁₁; where R₁₁ is hydrogen, or substituted or unsubstituted lower alkyl.

In one embodiment, in Formula IX,

R₁ is substituted or unsubstituted 4-morpholino or 4-pyridyl;

R₃ and R₄ are hydrogen, lower alkyl, substituted lower alkyl, or R₅;

R₅ is halogen or —OSO₂R₇;

R₇ is substituted or unsubstituted lower alkyl or aryl;

T is —CO—, —CS—, or —SO₂—;

X, Y, and Z are nitrogen atoms or CR₈, except that at least two of X, Y, and Z are nitrogen atoms; where R₈ is hydrogen or lower alkyl;

R₁₀ is hydrogen, amino, lower alkylamino, substituted lower alkylamino, hydroxy, lower alkoxy, substituted lower alkoxy, hydroxymethyl, carboxamido, or substituted carboxamido;

G and J are each independently a direct bond or —CH₂—; and

W is a direct bond; or oxygen, sulfur, or NR₁₁; where R₁₁ is hydrogen, or substituted or unsubstituted lower alkyl.

In yet another embodiment, the compound of Formula Ia has the structure of Formula X:

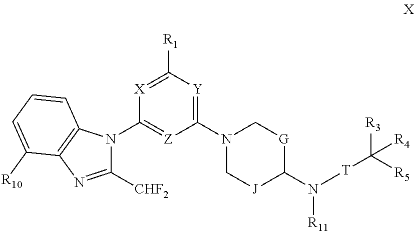

or an enantiomer, a mixture of enantiomers, or a mixture of two or more diastereomers thereof; or a pharmaceutically acceptable salt, solvate, hydrate, or prodrug thereof; wherein:

R₁, R₃, R₄, R₅, R₁₀, T, X, Y, and Z are each as defined herein;

R₁₁ is hydrogen, or substituted or unsubstituted lower alkyl; and

G and J are each independently a direct bond or —CH₂—.

In one embodiment, in Formula X,

R₁ is substituted or unsubstituted 4-morpholino or 4-pyridyl;

R₃ and R₄ are hydrogen, lower alkyl, substituted lower alkyl, or R₅;

R₅ is halogen or —OSO₂R₇;

R₇ is substituted or unsubstituted lower alkyl or aryl;

T is —CO—, —CS—, or —SO₂—;

X, Y, and Z are nitrogen atoms or CR₈, except that at least two of X, Y, and Z are nitrogen atoms; where R₈ is hydrogen or lower alkyl;

R₁₀ is hydrogen, amino, lower alkylamino, substituted lower alkylamino, hydroxy, lower alkoxy, substituted lower alkoxy, hydroxymethyl, carboxamido, or substituted carboxamido;

R₁₁ is hydrogen, or substituted or unsubstituted lower alkyl; and

G and J are each independently a direct bond or —CH₂—.

In yet another embodiment, the compound of Formula Ic has the structure of Formula XI:

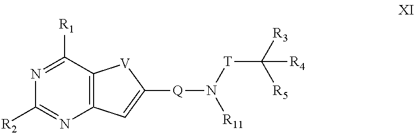

or an enantiomer, a mixture of enantiomers, or a mixture of two or more diastereomers thereof; or a pharmaceutically acceptable salt, solvate, hydrate, or prodrug thereof; wherein:

R₁, R₂, R₃, R₄, R₅, Q, and T are each as defined herein;

R₁₁ is hydrogen, or substituted or unsubstituted lower alkyl; and

V is oxygen or sulfur.

In one embodiment, in Formula XI,

R₁ is substituted or unsubstituted 4-morpholino or 4-pyridyl;

R₂ is substituted or unsubstituted aryl, heteroaryl, or heterocyclyl;

R₃ and R₄ are each independently hydrogen, lower alkyl, substituted lower alkyl, or R₅;

R₅ is halogen or —OSO₂R₇;

R₇ is substituted or unsubstituted lower alkyl or aryl;

Q is a linker group as defined herein;

T is —CO—, —CS—, or —SO₂—;

R₁₁ is hydrogen, or substituted or unsubstituted lower alkyl; and

V is oxygen or sulfur.

In yet another embodiment, the compound of Formula Ic has the structure of Formula XII:

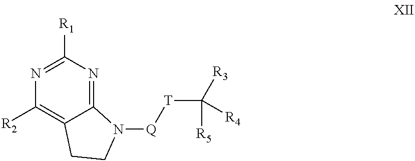

or an enantiomer, a mixture of enantiomers, or a mixture of two or more diastereomers thereof; or a pharmaceutically acceptable salt, solvate, hydrate, or prodrug thereof; wherein R₁, R₂, R₃, R₄, R₅, Q, and T are each as defined herein.

In one embodiment, in Formula XII,

R₁ is substituted or unsubstituted 4-morpholino or 4-pyridyl;

R₂ is substituted or unsubstituted aryl, heteroaryl, or heterocyclyl;

R₃ and R₄ are each independently hydrogen, lower alkyl, substituted lower alkyl, or R₅;

R₅ is halogen or —OSO₂R₇;

R₇ is substituted or unsubstituted lower alkyl or aryl;

Q is a linker group as defined herein; and

T is —CO—, —CS—, or —SO₂—.

In yet another embodiment, the compound of Formula Ic has the structure of Formula XIII:

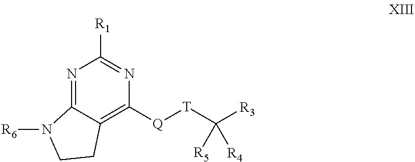

or an enantiomer, a mixture of enantiomers, or a mixture of two or more diastereomers thereof; or a pharmaceutically acceptable salt, solvate, hydrate, or prodrug thereof; wherein:

R₁, R₃, R₄, R₅, Q, and T are each as defined herein; and

R₆ is substituted or unsubstituted aryl or heteroaryl.

In one embodiment, in Formula XIII,

R₁ is substituted or unsubstituted 4-morpholino or 4-pyridyl;

R₃ and R₄ are each independently hydrogen, lower alkyl, substituted lower alkyl, or R₅;

R₅ is halogen or —OSO₂R₇;

R₆ is substituted or unsubstituted aryl or heteroaryl;

R₇ is substituted or unsubstituted lower alkyl or aryl;

Q is a linker group as defined herein; and

T is —CO—, —CS—, or —SO₂—.

In still another embodiment, the compound of Formula Ic has the structure of Formula XIV:

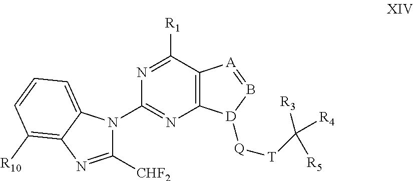

or an enantiomer, a mixture of enantiomers, or a mixture of two or more diastereomers thereof; or a pharmaceutically acceptable salt, solvate, hydrate, or prodrug thereof; wherein R₁, R₃, R₄, R₅, R₁₀, Q, T, A, B, and D are each as defined herein.

In one embodiment, in Formula XIV,

R₁ is substituted or unsubstituted 4-morpholino or 4-pyridyl;

R₃ and R₄ are hydrogen, lower alkyl, substituted lower alkyl, or R₅;

R₅ is halogen or —OSO₂R₇;

R₇ is substituted or unsubstituted lower alkyl or aryl;

Q is a linker group as defined herein;

T is —CO—, —CS—, or —SO₂—.

A, B, and D are each independently nitrogen or —CR₉;

R₉ is hydrogen or lower alkyl; and

R₁₀ is hydrogen, amino, lower alkylamino, substituted lower alkylamino, hydroxy, lower alkoxy, substituted lower alkoxy, hydroxymethyl, carboxamido, or substituted carboxamido.

The groups, R₁, R₂, R₃, R₄, R₅, R₆, R₇, R₈, R₉, R₁₀, R₁₁, A, B, D, E, G, J, Q, T, U, V, W, X, Y, and Z in Formula I, II, III, IV, V, VI, VII, VIII, IX, X, XI, XII, XIII, or XIV are further defined in the embodiments described herein. All combinations of the embodiments provided herein for such groups are within the scope of this disclosure.

In certain embodiments, each R₁ is independently C₆₋₁₄ aryl, optionally substituted as described herein. In certain embodiments, each R₁ is independently heteroaryl, optionally substituted as described herein. In certain embodiments, each R₁ is independently pyridyl, optionally substituted as described herein. In certain embodiments, each R₁ is independently 4-pyridyl, optionally substituted as described herein. In certain embodiments, each R₁ is independently heterocyclyl, optionally substituted as described herein. In certain embodiments, each R₁ is independently morpholino, optionally substituted as described herein. In certain embodiments, each R₁ is independently 4-morpholino, optionally substituted as described herein. In certain embodiments, each R₁ is independently 4-morpholino or 4-pyridyl.

In certain embodiments, each R₂ is independently C₆₋₁₄ aryl, optionally substituted as described herein. In certain embodiments, each R₂ is independently C₆₋₁₄ aryl, optionally substituted with one or more substituents, in one embodiment, one or two, substituents, each independently selected from hydroxyl and hydroxymethyl. In certain embodiments, each R₂ is independently phenyl, optionally substituted as described herein. In certain embodiments, each R₂ is independently hydroxyphenyl (e.g., 2-hydroxyphenyl, 3-hydroxyphenyl, or 4-hydroxyphenyl) or hydroxymethylphenyl (e.g., 2-hydroxymethylphenyl, 3-hydroxymethylphenyl, or 4-hydroxymethylphenyl). In certain embodiments, each R₂ is independently heteroaryl, optionally substituted as described herein. In certain embodiments, each R₂ is independently heteroaryl, optionally substituted with one or more substituents, in one embodiment, one or two, substituents, each independently selected from the group consisting of amino, difluoromethyl, hydroxyl, methoxy, ethoxy, N,N-dimethylaminopropoxy (e.g., 3-(N,N-dimethylamino)propoxy). In certain embodiments, each R₂ is independently heterocyclyl, optionally substituted as described herein. In certain embodiments, each R₂ is independently hydroxyphenyl, hydroxymethylphenyl, aminopyridyl, aminopyrimidyl, indazolyl, difluoromethyl-1H-benzimidazolyl, difluoromethyl-hydroxy-1H-benzimidazolyl, difluoromethyl-methoxy-1H-benzimidazolyl, difluoromethyl-ethoxy-1H-benzimidazolyl, or difluoromethyl-((N,N-dimethylamino)propoxy)-1H-benzimidazolyl. In certain embodiments, each R₂ is independently 3-hydroxyphenyl, 3-hydroxymethylphenyl, 2-amino-pyrid-5-yl, 2-amino-pyrimid-5-yl, indazol-4-yl, 2-difluoromethyl-1H-benzimidazolyl, 2-difluoromethyl-4-hydroxy-1H-benzimidazolyl, 2-difluoromethyl-4-methoxy-1H-benzimidazolyl, 2-difluoromethyl-4-ethoxy-1H-benzimidazolyl, or 2-difluoromethyl-4-(3-(N,N-dimethylamino)propoxy)-1H-benzimidazolyl.

In certain embodiments, each R₃ is independently hydrogen or lower alkyl (C₁₋₆). In certain embodiments, each R₃ is independently hydrogen or methyl. In certain embodiments, each R₃ is independently halogen. In certain embodiments, each R₃ is independently chloro. In certain embodiments, each R₃ is independently —OSO₂R₇. In certain embodiments, each R₃ is independently hydrogen, chloro, or methyl.

In certain embodiments, each R₄ is independently hydrogen or lower alkyl (C₁₋₆). In certain embodiments, each R₄ is independently hydrogen or methyl. In certain embodiments, each R₄ is independently halogen. In certain embodiments, each R₄ is independently chloro. In certain embodiments, each R₄ is independently —OSO₂R₇. In certain embodiments, each R₄ is independently hydrogen, chloro, or methyl.

In certain embodiments, each R₅ is independently halogen. In certain embodiments, each R₅ is independently chloro or bromo. In certain embodiments, each R₅ is independently —OSO₂R₇. In certain embodiments, each R₅ is independently chloro, bromo, or —OSO₂-methyl.

In certain embodiments, R₆ is C₃₋₇ cycloalkyl, optionally substituted as described herein. In certain embodiments, R₆ is C₆₋₁₄ aryl, optionally substituted as described herein. In certain embodiments, R₆ is C₆₋₁₄ aryl, optionally substituted with one or more substituents, in one embodiment, one or two, substituents, each independently selected from hydroxyl and hydroxymethyl. In certain embodiments, R₆ is phenyl, optionally substituted as described herein. In certain embodiments, R₆ is hydroxyphenyl (e.g., 2-hydroxyphenyl, 3-hydroxyphenyl, or 4-hydroxyphenyl) or hydroxymethylphenyl (e.g., 2-hydroxymethylphenyl, 3-hydroxymethylphenyl, or 4-hydroxymethylphenyl). In certain embodiments, R₆ is heteroaryl, optionally substituted as described herein. In certain embodiments, R₆ is heteroaryl, optionally substituted with one or more substituents, in one embodiment, one or two, substituents, each independently selected from the group consisting of amino, difluoromethyl, hydroxyl, methoxy, ethoxy, N,N-dimethylaminopropoxy (e.g., 3-(N,N-dimethylamino)propoxy). In certain embodiments, R₆ is heterocyclyl, optionally substituted as described herein. In certain embodiments, R₆ is hydroxyphenyl, hydroxymethylphenyl, aminopyridyl, aminopyrimidyl, indazolyl, difluoromethyl-1H-benzimidazolyl, difluoromethyl-hydroxy-1H-benzimidazolyl, difluoromethyl-methoxy-1H-benzimidazolyl, difluoromethyl-ethoxy-1H-benzimidazolyl, or difluoromethyl-((N,N-dimethylamino)propoxy)-1H-benzimidazolyl. In certain embodiments, R₆ is 3-hydroxyphenyl, 3-hydroxymethylphenyl, 2-amino-pyrid-5-yl, 2-amino-pyrimid-5-yl, indazol-4-yl, 2-difluoromethyl-1H-benzimidazolyl, 2-difluoromethyl-4-hydroxy-1H-benzimidazolyl, 2-difluoromethyl-4-methoxy-1H-benzimidazolyl, 2-difluoromethyl-4-ethoxy-1H-benzimidazolyl, or 2-difluoromethyl-4-(3-(N,N-dimethylamino)propoxy)-1H-benzimidazolyl.

In certain embodiments, R₇ is lower alkyl (C₁₋₆), optionally substituted as described herein. In certain embodiments, R₇ is methyl, fluoromethyl, difluormethyl, or trifluoromethyl. In certain embodiments, R₇ is C₂₋₆ alkenyl, optionally substituted as described herein. In certain embodiments, R₇ is C₂₋₆ alkynyl, optionally substituted as described herein. In certain embodiments, R₇ is C₃₋₇ cycloalkyl, optionally substituted as described herein. In certain embodiments, R₇ is C₆₋₁₄ aryl, optionally substituted as described herein. In certain embodiments, R₇ is phenyl, optionally substituted as described herein. In certain embodiments, R₇ is heteroaryl, optionally substituted as described herein. In certain embodiments, R₇ is heterocyclyl, optionally substituted as described herein.

In certain embodiments, R₈ is hydrogen or lower alkyl (C₁₋₆).

In certain embodiments, R₉ is hydrogen, halogen, cyano, amino, or hydroxyl. In certain embodiments, R₉ is lower alkyl (C₁₋₆), optionally substituted as described herein. In certain embodiments, R₉ is fluoromethyl, difluoromethyl, or trifluoromethyl. In certain embodiments, R₉ is lower alkylamino or lower alkoxy, each optionally substituted as described herein.

In certain embodiments, R₁₀ is hydrogen, amino, or hydroxyl. In certain embodiments, R₁₀ is lower alkyl (C₁₋₆), lower alkylamino, di(lower alkyl)amino, lower alkoxy, or carboxamido, each optionally substituted as described herein. In certain embodiments, R₁₀ is hydrogen, hydroxy, methoxy, ethoxy, or 3-(N,N-dimethylamino)propoxy.

In certain embodiments, G is a direct bond. In certain embodiments, G is —CH₂—.

In certain embodiments, J is a direct bond. In certain embodiments, J is —CH₂—.

In certain embodiments, Q is absent. In certain embodiments, Q is substituted or unsubstituted heterocyclylene. In certain embodiments, Q is a divalent linker selected from the group consisting of azetidinyleneamino (e.g., azetidinylene-2-amino, (R)-azetidinylene-2-amino, (S)-azetidinylene-2-amino, azetidinylene-3-amino, (R)-azetidinylene-3-amino, and (S)-azetidinylene-3-amino), azetidinylene(methylamino) (e.g., azetidinylene-2-methyamino, (R)-azetidinylene-2-methyamino, (S)-azetidinylene-2-methyamino, azetidinylene-3-methyamino, (R)-azetidinylene-3-methyamino, and (S)-azetidinylene-3-methyamino), piperidyleneoxy (e.g., 2-piperidyleneoxy, 2(R)-piperidyleneoxy, 2(S)-piperidyleneoxy, 3-piperidyleneoxy, 3(R)-piperidyleneoxy, 3(S)-piperidyleneoxy, and 4-piperidyleneoxy), piperazinylene (e.g., 1,2-piperazinylene, 1,3-piperazinylene, and 1,4-piperazinylene), piperidylene (1,2-piperidylene, 1,3-piperidylene, and 1,4-piperidylene), piperidyleneamino (e.g., piperidylene-2-amino, (R)-piperidylene-2-amino, (S)-piperidylene-2-amino, piperidylene-3-amino, (R)-piperidylene-3-amino, (S)-piperidylene-3-amino, and piperidylene-4-amino), piperidylene(methylamino) (e.g., piperidylene-2-methylamino, (R)-piperidylene-2-methylamino, (S)-piperidylene-2-methylamino, piperidylene-3-methylamino, (R)-piperidylene-3-methylamino, (S)-piperidylene-3-methylamino, and piperidylene-4-methylamino), pyrrolidinyleneamino (e.g., pyrrolidinylene-2-amino, (R)-pyrrolidinylene-2-amino, (S)-pyrrolidinylene-2-amino, pyrrolidinylene-3-amino, (R)-pyrrolidinylene-3-amino, and (S)-pyrrolidinylene-3-amino), pyrrolidinylene(methylamino) (e.g., pyrrolidinylene-2-methylamino, (R)-pyrrolidinylene-2-methylamino, (S)-pyrrolidinylene-2-methylamino, pyrrolidinylene-3-methylamino, (R)-pyrrolidinylene-3-methylamino, and (S)-pyrrolidinylene-3-methylamino), and piperidylthio (e.g., 2-piperidylenethio, 2(R)-piperidylenethio, 2(S)-piperidylenethio, 3-piperidylenethio, 3(R)-piperidylenethio, 3(S)-piperidylenethio, and 4-piperidylenethio). In certain embodiments, Q is a divalent linker selected from the group consisting of azetidinylene-4-amino, azetidinylene-4-methylamino, piperidylene-4-oxy, 1,4-piperazinylene, 1,4-piperidylene, piperidylene-3-amino, (R)-piperidylene-3-amino, (S)-piperidylene-3-amino, piperidylene-3-methylamino, (R)-piperidylene-3-methylamino, (S)-piperidylene-3-methylamino, piperidylene-4-amino, piperidylene-4-methylamino, pyrrolidinylene-3-amino, (R)-pyrrolidinylene-3-amino, (S)-pyrrolidinylene-3-amino, pyrrolidinylene-3-methylamino, (R)-pyrrolidinylene-3-methylamino, (S)-pyrrolidinylene-3-methylamino, and piperidylene-4-thio.

In certain embodiments, T is —CO—. In certain embodiments, T is —CS—. In certain embodiments, T is —SO₂—.

In certain embodiments, U is N. In certain embodiments, U is CH.

In certain embodiments, V is oxygen. In certain embodiments, V is sulfur.

In certain embodiments, W is a direct bond, oxygen, or sulfur. In certain embodiments, W is NR₁₁; where R₁₁ is as defined herein.

In certain embodiments, X is nitrogen or CR₈. In certain embodiments, X is nitrogen or CH. In certain embodiments, Y is nitrogen or CR₈. In certain embodiments, Y is nitrogen or CH. In certain embodiments, Z is nitrogen or CR₈. In certain embodiments, Z is nitrogen or CH.

In certain embodiments, X, Y, and Z are nitrogen. In certain embodiments, X and Y are nitrogen, and Z is CH. In certain embodiments, X and Z are nitrogen, and Y is CH. In certain embodiments, Y and Z are nitrogen, and X is CH.

In certain embodiments, each A is independently a direct bond. In certain embodiments, each A is independently a nitrogen, oxygen, or sulfur atom. In certain embodiments, each A is independently CR₉, where R₉ is as defined herein. In certain embodiments, each A is independently CR₉, where R₉ is hydrogen, lower alkyl, or halogen.

In certain embodiments, each B is independently a direct bond. In certain embodiments, each B is independently a nitrogen, oxygen, or sulfur atom. In certain embodiments, each B is independently CR₉, where R₉ is as defined herein. In certain embodiments, each B is independently CR₉, where R₉ is hydrogen, lower alkyl, or halogen.

In certain embodiments, each D is independently a direct bond. In certain embodiments, each D is independently a nitrogen, oxygen, or sulfur atom. In certain embodiments, each D is independently CR₉, where R₉ is as defined herein. In certain embodiments, each D is independently CR₉, where R₉ is hydrogen, lower alkyl, or halogen.

In certain embodiments, each E is independently a direct bond. In certain embodiments, each E is independently a nitrogen, oxygen, or sulfur atom. In certain embodiments, each E is independently CR₉, where R₉ is as defined herein. In certain embodiments, each E is independently CR₉, where R₉ is hydrogen, lower alkyl, or halogen.

In one embodiment, provided herein is a compound selected from:
3-[4-[4-(chloroacetyl)-1-piperazinyl]-6-(4-morpholinyl)-1,3,5-triazin-2-yl]phenol;
1-[4-[4-(chloroacetyl)-1-piperazinyl]-6-(4-morpholinyl)-1,3,5-triazin-2-yl]-2-(difluoromethyl)-4-ethoxy-1H-benzimidazole;
1-[4-[4-(chloroacetyl)-1-piperazinyl]-6-(4-morpholinyl)-1,3,5-triazin-2-yl]-2-(difluoromethyl)-1H-benzimidazole;
1-[4-[4-(chloroacetyl)-1-piperazinyl]-6-(4-morpholinyl)-1,3,5-triazin-2-yl]-2-(difluoromethyl)-4-methoxy-1H-benzimidazole;
1-[4-[4-(chloroacetyl)-1-piperazinyl]-6-(4-morpholinyl)-1,3,5-triazin-2-yl]-2-(difluoromethyl)-1H-benzimidazol-4-ol;
1-[4-[4-(chloroacetyl)-1-piperazinyl]-6-(4-pyridinyl)-1,3,5-triazin-2-yl]-2-(difluoromethyl)-4-methoxy-1H-benzimidazole;
1-[4-[4-(dichloroacetyl)-1-piperazinyl]-6-(4-morpholinyl)-1,3,5-triazin-2-yl]-2-(difluoromethyl)-4-methoxy-1H-benzimidazole;
2-(difluoromethyl)-1-{4-(4-morpholinyl)-6-[4-(trichloroacetyl)-1-piperazinyl]-1,3,5-triazin-2-yl}-1H-benzimidazol-4-yl methyl ether;
2-{4-[4-[2-(difluoromethyl)-4-methoxy-1H-benzimidazol-1-yl]-6-(4-morpholinyl)-1,3,5-triazin-2-yl]-1-piperazinyl}-2-oxoethyl methanesulfonate;
1-[4-[4-(2-chloropropanoyl)-1-piperazinyl]-6-(4-morpholinyl)-1,3,5-triazin-2-yl]-2-(difluoromethyl)-4-methoxy-1H-benzimidazole;
1-[4-{4-[(chloromethyl)sulfonyl]-1-piperazinyl}-6-(4-morpholinyl)-1,3,5-triazin-2-yl]-2-(difluoromethyl)-4-methoxy-1H-benzimidazole;
1-[4-{4-[(bromomethyl)sulfonyl]-1-piperazinyl}-6-(4-morpholinyl)-1,3,5-triazin-2-yl]-2-(difluoromethyl)-4-methoxy-1H-benzimidazole;
N-(3-{[1-[4-{4-[(chloromethyl)sulfonyl]-1-piperazinyl}-6-(4-morpholinyl)-1,3,5-triazin-2-yl]-2-(difluoromethyl)-1H-benzimidazol-4-yl]oxy}propyl)-N,N-dimethylamine;
1-[4-{4-[(chloromethyl)sulfonyl]-1-piperazinyl}-6-(4-morpholinyl)-2-pyrimidinyl]-2-(difluoromethyl)-4-methoxy-1H-benzimidazole;
1-[6-{4-[(chloromethyl)sulfonyl]-1-piperazinyl}-2-(4-morpholinyl)-4-pyrimidinyl]-2-(difluoromethyl)-4-methoxy-1H-benzimidazole;
1-[2-{4-[(chloromethyl)sulfonyl]-1-piperazinyl}-6-(4-morpholinyl)-4-pyrimidinyl]-2-(difluoromethyl)-4-methoxy-1H-benzimidazole;
1-[4-[1-(chloroacetyl)-4-piperidinyl]-6-(4-morpholinyl)-1,3,5-triazin-2-yl]-2-(difluoromethyl)-4-methoxy-1H-benzimidazole;
N-[1-(chloroacetyl)-4-piperidinyl]-4-[2-(difluoromethyl)-4-methoxy-1H-benzimidazol-1-yl]-6-(4-morpholinyl)-1,3,5-triazin-2-amine;
N-[1-(chloroacetyl)-4-piperidinyl]-4-[2-(difluoromethyl)-4-methoxy-1H-benzimidazol-1-yl]-N-methyl-6-(4-morpholinyl)-1,3,5-triazin-2-amine;
N-[1-(chloroacetyl)-4-piperidinyl]-4-{2-(difluoromethyl)-4-[3-(dimethylamino)propoxy]-1H-benzimidazol-1-yl}-N-methyl-6-(4-morpholinyl)-1,3,5-triazin-2-amine;
N-{1-[(chloromethyl)sulfonyl]-4-piperidinyl}-4-[2-(difluoromethyl)-4-methoxy-1H-benzimidazol-1-yl]-N-methyl-6-(4-morpholinyl)-1,3,5-triazin-2-amine;
2-chloro-N-{1-[4-[2-(difluoromethyl)-4-methoxy-1H-benzimidazol-1-yl]-6-(4-morpholinyl)-1,3,5-triazin-2-yl]-4-piperidinyl}acetamide;
2-chloro-N-{1-[4-[2-(difluoromethyl)-4-methoxy-1H-benzimidazol-1-yl]-6-(4-morpholinyl)-1,3,5-triazin-2-yl]-4-piperidinyl}-N-methylacetamide;
2-chloro-N-{1-[4-{2-(difluoromethyl)-4-[3-(dimethylamino)propoxy]-1H-benzimidazol-1-yl}-6-(4-morpholinyl)-1,3,5-triazin-2-yl]-4-piperidinyl}-N-methylacetamide;
2-chloro-N-{(3R)-1-[4-[2-(difluoromethyl)-4-methoxy-1H-benzimidazol-1-yl]-6-(4-morpholinyl)-1,3,5-triazin-2-yl]pyrrolidinyl}acetamide;
2-chloro-N-{(3S)-1-[4-[2-(difluoromethyl)-4-methoxy-1H-benzimidazol-1-yl]-6-(4-morpholinyl)-1,3,5-triazin-2-yl]pyrrolidinyl}acetamide;
2-chloro-N-{(3R)-1-[4-[2-(difluoromethyl)-4-methoxy-1H-benzimidazol-1-yl]-6-(4-morpholinyl)-1,3,5-triazin-2-yl]pyrrolidinyl}-N-methylacetamide;
2-chloro-N-{(3S)-1-[4-[2-(difluoromethyl)-4-methoxy-1H-benzimidazol-1-yl]-6-(4-morpholinyl)-1,3,5-triazin-2-yl]pyrrolidinyl}-N-methylacetamide;
N-[(3R)-1-(chloro acetyl)pyrrolidinyl]-4-[2-(difluoromethyl)-4-methoxy-1H-benzimidazol-1-yl]-6-(4-morpholinyl)-1,3,5-triazin-2-amine;
N-[(3S)-1-(chloro acetyl)pyrrolidinyl]-4-[2-(difluoromethyl)-4-methoxy-1H-benzimidazol-1-yl]-6-(4-morpholinyl)-1,3,5-triazin-2-amine;
N-[(3R)-1-(chloro acetyl)pyrrolidinyl]-4-[2-(difluoromethyl)-4-methoxy-1H-benzimidazol-1-yl]-N-methyl-6-(4-morpholinyl)-1,3,5-triazin-2-amine;
N-[(3S)-1-(chloro acetyl)pyrrolidinyl]-4-[2-(difluoromethyl)-4-methoxy-1H-benzimidazol-1-yl]-N-methyl-6-(4-morpholinyl)-1,3,5-triazin-2-amine;
2-chloro-N-{(3R)-1-[4-[2-(difluoromethyl)-4-methoxy-1H-benzimidazol-1-yl]-6-(4-morpholinyl)-1,3,5-triazin-2-yl]piperidinyl}acetamide;
2-chloro-N-{(3S)-1-[4-[2-(difluoromethyl)-4-methoxy-1H-benzimidazol-1-yl]-6-(4-morpholinyl)-1,3,5-triazin-2-yl]piperidinyl}acetamide;
2-chloro-N-{(3R)-1-[4-[2-(difluoromethyl)-4-methoxy-1H-benzimidazol-1-yl]-6-(4-morpholinyl)-1,3,5-triazin-2-yl]piperidinyl}-N-methylacetamide;
2-chloro-N-{(3S)-1-[4-[2-(difluoromethyl)-4-methoxy-1H-benzimidazol-1-yl]-6-(4-morpholinyl)-1,3,5-triazin-2-yl]piperidinyl}-N-methylacetamide;
N-[(3R)-1-(chloroacetyl)piperidinyl]-4-[2-(difluoromethyl)-4-methoxy-1H-benzimidazol-1-yl]-N-methyl-6-(4-morpholinyl)-1,3,5-triazin-2-amine;
N-[(3S)-1-(chloroacetyl)piperidinyl]-4-[2-(difluoromethyl)-4-methoxy-1H-benzimidazol-1-yl]-6-(4-morpholinyl)-1,3,5-triazin-2-amine;
N-[(3R)-1-(chloroacetyl)piperidinyl]-4-[2-(difluoromethyl)-4-methoxy-1H-benzimidazol-1-yl]-N-methyl-6-(4-morpholinyl)-1,3,5-triazin-2-amine;
N-[(3S)-1-(chloroacetyl)piperidinyl]-4-[2-(difluoromethyl)-4-methoxy-1H-benzimidazol-1-yl]-N-methyl-6-(4-morpholinyl)-1,3,5-triazin-2-amine;
2-chloro-N-{1-[4-[2-(difluoromethyl)-4-methoxy-1H-benzimidazol-1-yl]-6-(4-morpholinyl)-1,3,5-triazin-2-yl]-3-azetidinyl}acetamide;
2-chloro-N-{1-[4-[2-(difluoromethyl)-4-methoxy-1H-benzimidazol-1-yl]-6-(4-morpholinyl)-1,3,5-triazin-2-yl]-3-azetidinyl}-N-methylacetamide;
N-[1-(chloro acetyl)-3-azetidinyl]-4-[2-(difluoromethyl)-4-methoxy-1H-benzimidazol-1-yl]-6-(4-morpholinyl)-1,3,5-triazin-2-amine;
N-[1-(chloro acetyl)-3-azetidinyl]-4-[2-(difluoromethyl)-4-methoxy-1H-benzimidazol-1-yl]-N-methyl-6-(4-morpholinyl)-1,3,5-triazin-2-amine;
1-[4-{[1-(chloroacetyl)-4-piperidinyl]oxy}-6-(4-morpholinyl)-1,3,5-triazin-2-yl]-2-(difluoromethyl)-4-methoxy-1H-benzimidazole;
3-{[1-[4-{[1-(chloroacetyl)-4-piperidinyl]oxy}-6-(4-morpholinyl)-1,3,5-triazin-2-yl]-2-(difluoromethyl)-1H-benzimidazol-4-yl]oxy}-N,N-dimethyl-1-propanamine;
3-{[1-[4-({1-[(chloromethyl)sulfonyl]-4-piperidinyl}oxy)-6-(4-morpholinyl)-1,3,5-triazin-2-yl]-2-(difluoromethyl)-1H-benzimidazol-4-yl]oxy}-N,N-dimethyl-1-propanamine; and
1-{1-[(chloromethyl)sulfonyl]-4-piperidinyl}-6-[2-(difluoromethyl)-4-methoxy-1H-benzimidazol-1-yl]-4-(4-morpholinyl)-1H-pyrazolo[3,4-d]pyrimidine;
   and enantiomers, mixtures of enantiomers, or mixtures of two or more diastereomers thereof;
   and pharmaceutically acceptable salts, solvates, hydrates, and prodrugs thereof.

In another embodiment, provided herein is a compound selected from:
1-[4-[4-(chloroacetyl)-1-piperazinyl]-6-(4-pyridinyl)-1,3,5-triazin-2-yl]-2-(difluoromethyl)-1H-benzimidazole;
1-[4-[4-(chloroacetyl)-1-piperazinyl]-6-(4-pyridinyl)-1,3,5-triazin-2-yl]-2-(difluoromethyl)-4-ethoxy-1H-benzimidazole;
1-[4-[4-(chloroacetyl)-1-piperazinyl]-6-(4-pyridinyl)-1,3,5-triazin-2-yl]-2-(difluoromethyl)-1H-benzimidazol-4-ol;
3-[4-[4-(chloroacetyl)-1-piperazinyl]-6-(4-pyridinyl)-1,3,5-triazin-2-yl]phenol;
[3-[4-[4-(chloroacetyl)-1-piperazinyl]-6-(4-morpholinyl)-1,3,5-triazin-2-yl]phenyl]methanol;
[3-[4-[4-(chloroacetyl)-1-piperazinyl]-6-(4-pyridinyl)-1,3,5-triazin-2-yl]phenyl]methanol;
5-[4-[4-(chloroacetyl)-1-piperazinyl]-6-(4-morpholinyl)-1,3,5-triazin-2-yl]-2-pyridinamine;
5-[4-[4-(chloroacetyl)-1-piperazinyl]-6-(4-pyridinyl)-1,3,5-triazin-2-yl]-2-pyridinamine;
5-[4-[4-(chloroacetyl)-1-piperazinyl]-6-(4-morpholinyl)-1,3,5-triazin-2-yl]-2-pyrimidinamine;
5-[4-[4-(chloroacetyl)-1-piperazinyl]-6-(4-pyridinyl)-1,3,5-triazin-2-yl]-2-pyrimidinamine;
4-[4-[4-(chloroacetyl)-1-piperazinyl]-6-(4-morpholinyl)-1,3,5-triazin-2-yl]-1H-indazole;
4-[4-[4-(chloroacetyl)-1-piperazinyl]-6-(4-pyridinyl)-1,3,5-triazin-2-yl]-1H-indazole;
1-[4-[1-(chloroacetyl)-4-piperidinyl]-6-(4-morpholinyl)-1,3,5-triazin-2-yl]-2-(difluoromethyl)-1H-benzimidazole;
1-[4-[1-(chloroacetyl)-4-piperidinyl]-6-(4-morpholinyl)-1,3,5-triazin-2-yl]-2-(difluoromethyl)-1H-benzimidazol-4-ol;
1-[4-[1-(chloroacetyl)-4-piperidinyl]-6-(4-morpholinyl)-1,3,5-triazin-2-yl]-2-(difluoromethyl)-4-ethoxy-1H-benzimidazole;
N-[1-(chloroacetyl)-4-piperidinyl]-4-[2-(difluoromethyl)-1H-benzimidazol-1-yl]-6-(4-morpholinyl)-1,3,5-triazin-2-amine;
1-[4-{[1-(chloroacetyl)-4-piperidinyl]amino}-6-(4-morpholinyl)-1,3,5-triazin-2-yl]-2-(difluoromethyl)-1H-benzimidazol-4-ol;
N-[1-(chloroacetyl)-4-piperidinyl]-4-[2-(difluoromethyl)-4-ethoxy-1H-benzimidazol-1-yl]-6-(4-morpholinyl)-1,3,5-triazin-2-amine;
1-[4-{[1-(chloroacetyl)-4-piperidinyl]oxy}-6-(4-morpholinyl)-1,3,5-triazin-2-yl]-2-(difluoromethyl)-1H-benzimidazole;
1-[4-{[1-(chloroacetyl)-4-piperidinyl]oxy}-6-(4-morpholinyl)-1,3,5-triazin-2-yl]-2-(difluoromethyl)-1H-benzimidazol-4-ol;
1-[4-{[1-(chloroacetyl)-4-piperidinyl]oxy}-6-(4-morpholinyl)-1,3,5-triazin-2-yl]-2-(difluoromethyl)-4-ethoxy-1H-benzimidazole;
1-[4-{[1-(chloro acetyl)-4-piperidinyl]sulfanyl}-6-(4-morpholinyl)-1,3,5-triazin-2-yl]-2-(difluoromethyl)-1H-benzimidazole;
1-[4-{[1-(chloro acetyl)-4-piperidinyl]sulfanyl}-6-(4-morpholinyl)-1,3,5-triazin-2-yl]-2-(difluoromethyl)-1H-benzimidazol-4-ol;
1-[4-{[1-(chloro acetyl)-4-piperidinyl]sulfanyl}-6-(4-morpholinyl)-1,3,5-triazin-2-yl]-2-(difluoromethyl)-4-methoxy-1H-benzimidazole;
1-[4-{[1-(chloro acetyl)-4-piperidinyl]sulfanyl}-6-(4-morpholinyl)-1,3,5-triazin-2-yl]-2-(difluoromethyl)-4-ethoxy-1H-benzimidazole;
2-chloro-N-{1-[4-[2-(difluoromethyl)-1H-benzimidazol-1-yl]-6-(4-morpholinyl)-1,3,5-triazin-2-yl]-4-piperidinyl}acetamide;
2-chloro-N-{1-[4-[2-(difluoromethyl)-4-hydroxy-1H-benzimidazol-1-yl]-6-(4-morpholinyl)-1,3,5-triazin-2-yl]-4-piperidinyl}acetamide; and
2-chloro-N-{1-[4-[2-(difluoromethyl)-4-ethoxy-1H-benzimidazol-1-yl]-6-(4-morpholinyl)-1,3,5-triazin-2-yl]-4-piperidinyl}acetamide;
   and enantiomers, mixtures of enantiomers, or mixtures of two or more diastereomers thereof;
   and pharmaceutically acceptable salts, solvates, hydrates, and prodrugs thereof.

The compounds provided herein are intended to encompass all possible stereoisomers, unless a particular stereochemistry is specified. Where the compound provided herein contains an alkenyl or alkenylene group, the compound may exist as one or mixture of geometric cis/trans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contain an aromatic moiety. It follows that a single compound may exhibit more than one type of isomerism.

The compounds provided herein may be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When the compound provided herein contains an acidic or basic moiety, it may also be provided as a pharmaceutically acceptable salt (See, Berge et al., J. Pharm. Sci. 1977, 66, 1-19; and “Handbook of Pharmaceutical Salts, Properties, and Use,” Stahl and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

In certain embodiments, the compounds provided herein are pharmacologically acceptable salts of the compounds with one or more of hydrochloric, sulfuric, phosphoric, acetic, citric, oxalic, malonic, salicylic, malic, fumaric, succinic, ascorbic, maleic, methanesulfonic, and isoethonic acids; or with one or more of potassium carbonate, sodium or potassium hydroxide, ammonia, triethylamine, and triethanolamine.

The compound provided herein may also be provided as a prodrug, which is a functional derivative of the compound, for example, of Formula I and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; and Waller et al., Br. J. Clin. Pharmac. 1989, 28, 497-507.

In certain embodiments, provided herein is a phosphate, carboxylic acid, amino acid ester prodrug of the compound provided herein.

In certain embodiments, the compounds provided herein are reversible inhibitors of PI3K. In certain embodiments, the compounds provided herein are irreversible inhibitors of PI3K. In certain embodiments, the compounds provided herein are selective reversible inhibitors of PI3K isoforms. In certain embodiments, the compounds provided herein are selective irreversible inhibitors of PI3K isoforms.

In certain embodiments, the compounds provided herein are reversible inhibitors of p110α. In certain embodiments, the compounds provided herein are irreversible inhibitors of p110α. In certain embodiments, the compounds provided herein are selective reversible inhibitors of p110α. In certain embodiments, the compounds provided herein are selective irreversible inhibitors of p110α.

Without being bound by any theory, it is believed that, in certain embodiments, the compounds provided herein interact with the His-855, which is unique to p110α. Without being bound by any theory, it is believed that, in certain embodiments, the compounds provided herein react with the His-855, which is unique to p110α. Without being bound by any theory, it is believed that, in certain embodiments, the compounds provided herein alkylate the His-855, which is unique to p110α.

Without being bound by any theory, it is believed that, in certain embodiments, the compounds provided herein are adapted to irreversibly inhibit the p110α isoform of PI3K. Without being bound by any theory, it is believed that, in certain embodiments, the compounds provided herein are adapted to target the His-855 group which is considered to form part of the ATP binding pocket of the p110α isoform of PI3K, but not of the other isoforms. Without being bound by any theory, it is believed that, in certain embodiments, by targeting the His-855 of p110α, the compounds provided herein selectively and irreversibly inhibit this PI3K isoform.

Irreversible inhibition of an enzyme target has a number of potential advantages: e.g., (a) kinase inhibitors that shut down the ATP site by reversible competitive blockade of ATP have to bind very tightly to the enzyme and/or maintain high plasma levels for prolonged periods, in order to compete with ATP binding, since ATP levels in cells are high; (b) the enzyme is shut down permanently, and the pathway is only reactivated upon resynthesis of the enzyme, which may take some time; (c) it allows longer times between doses, for a more achievable dosage regime; (d) it provides an additional mechanism for selectivity, in one embodiment, between different isoforms of an enzyme.

### Methods of Synthesis

In one embodiment, provided herein is a method of making a compound of Formula Ia, Ib, Ic, or Id as defined herein, which comprises the step of coupling a substituted pyrimidine or triazine intermediate with a suitable species that contains an alkylhalide or alkylsulfonate.

In certain embodiments, provided herein is a method of making a compound of Formula Ia, Ib, Ic, or Id as defined herein, which comprises the step of coupling a substituted pyrimidine or triazine intermediate with a suitable species. In one embodiment, the suitable species contains an alkylhalide or alkylsulfonate that is capable of selectively alkylating the His-855 of the p110α subunit of PI3K.

In certain embodiments, when the linker group Q of a compound of Formula I contains an NH group, the method comprises the step of reacting the pyrimidine or triazine intermediate with a suitable acylating species. In certain embodiments, the method comprises the step of reacting a halo substituted pyrimidine or triazine intermediate with a suitable acylated species.

The compounds provided herein can be prepared by a number of different methods, and the following examples are only representative and do not exclude other related procedures.

In one embodiment, when the linker group Q contains an amino substituent, the compounds of Formula Ia, Ib, Ic, or Id are prepared by reaction with an acylating species (e.g., acylhalide) or acid equivalent in the presence of a coupling reagent (Method A):

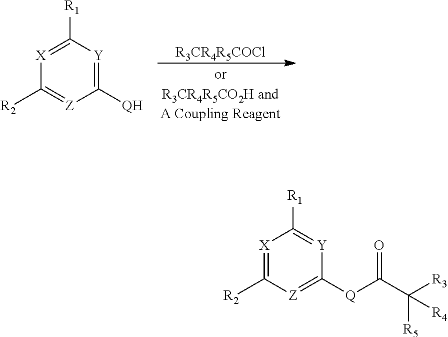

wherein R₁, R₂, R₃, R₄, R₅, Q, X, Y, and Z are each as defined herein.

The example shown in Scheme 1 is provided as an illustration of Method A.

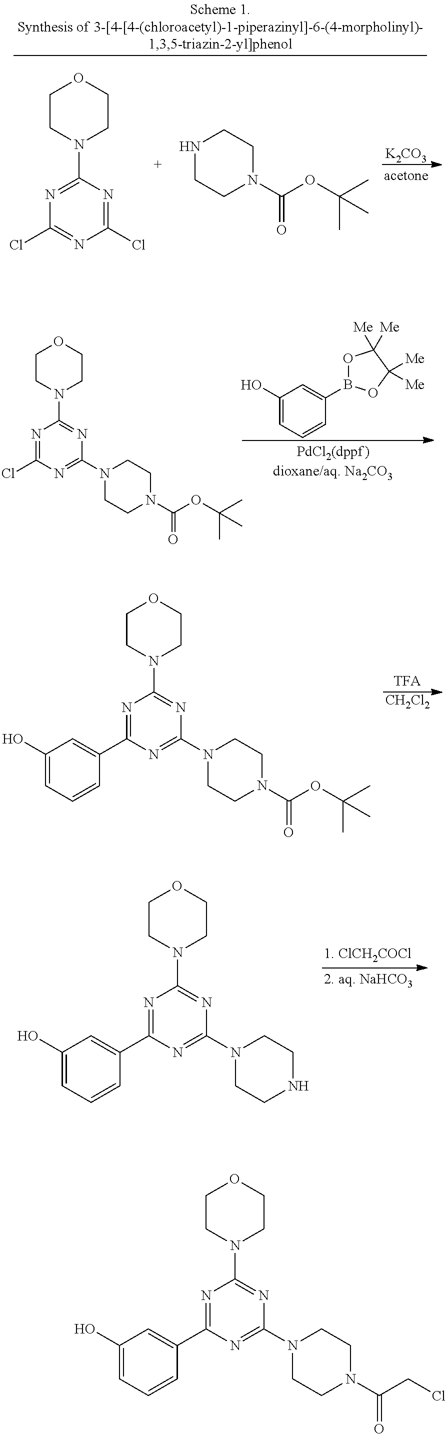

In another embodiment, the compounds of Formula Ia, Ib, Ic, or Id are prepared by a halogen displacement reaction on a 2- or 4-halopyrimidine or 2-halo-1,3,5-triazine derivative by a previously acylated species (Method B):

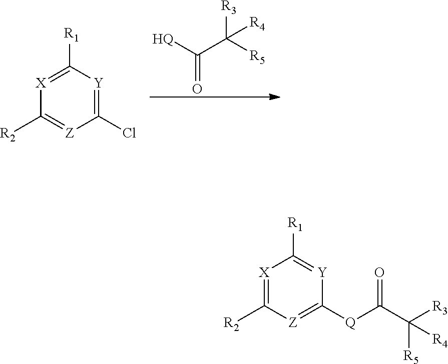

The example shown in Scheme 2 is provided as an illustration of Method B.

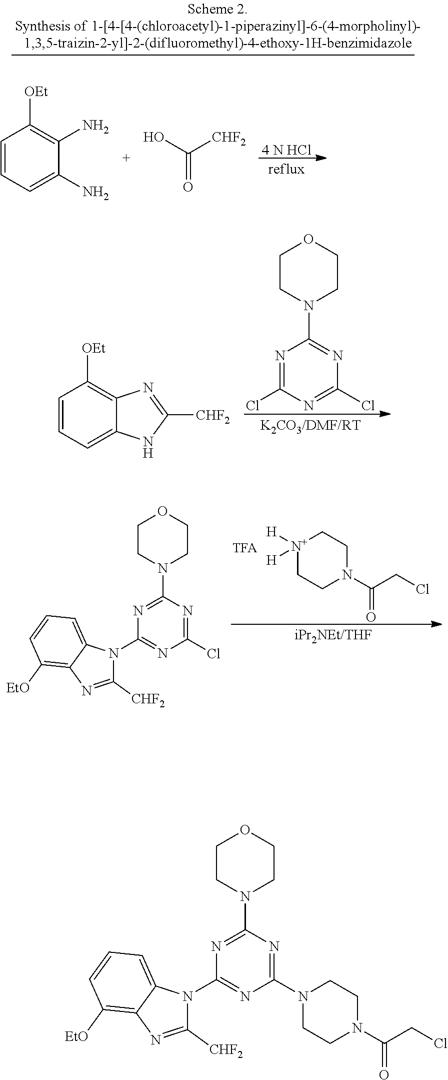

Table 1 gives details of representative compounds provided herein, and preparable by the methods outlined in Schemes 1 and 2.

TABLE 1
Examples of representative compounds provided herein^{a}
II

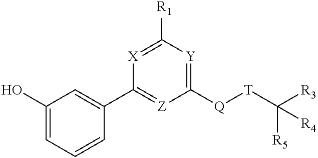

VII

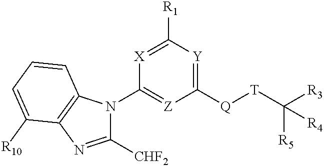

XIV

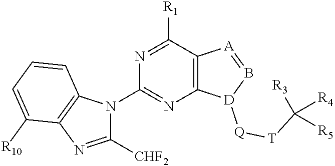

| Example | Type | X | Y | Z | R₁ | Q-T-CR₃R₄R₅ | R₁₀ | MW |
|---|---|---|---|---|---|---|---|---|
| 1 | II | N | N | N | 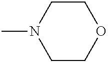 | 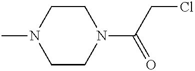 | — | 418.89 |
| 2 | VII | N | N | N | 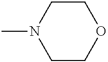 | 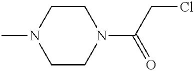 | OEt | 536.97 |
| 3 | VII | N | N | N | 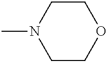 | 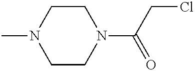 | H | 492.92 |
| 4 | VII | N | N | N | 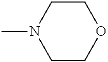 | 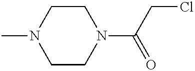 | OMe | 522.95 |
| 5 | VII | N | N | N | 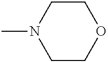 | 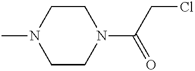 | OH | 508.92 |
| 6 | VII | N | N | N | 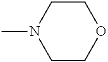 | 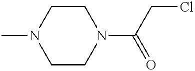 | OMe | 514.92 |
| 7 | VII | N | N | N | 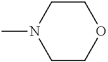 | 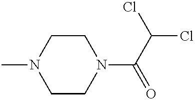 | OMe | 557.38 |
| 8 | VII | N | N | N | 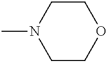 | 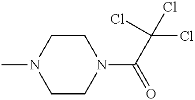 | OMe | 591.83 |
| 9 | VII | N | N | N | 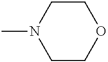 | 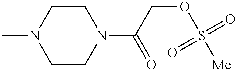 | OMe | 582.58 |
| 10 | VII | N | N | N | 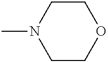 | 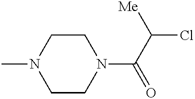 | OMe | 536.96 |
| 11 | VII | N | N | N | 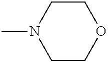 | 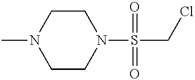 | OMe | 558.99 |
| 12 | VII | N | N | N | 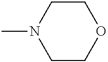 | 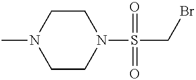 | OMe | 603.44 |
| 13 | VII | N | N | N | 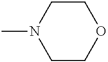 | 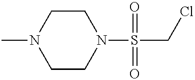 | O(CH₂)₃NMe₂ | 630.11 |
| 14 | VII | N | CH | N | 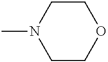 | 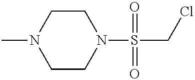 | OMe | 558.00 |
| 15 | VII | N | N | CH | 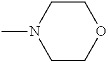 | 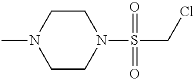 | OMe | 558.00 |
| 16 | VII | CH | N | N | 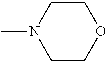 | 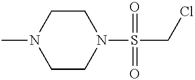 | OMe | 558.00 |
| 17 | VII | N | N | N | 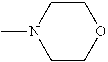 | 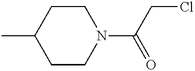 | OMe | 521.95 |
| 18 | VII | N | N | N | 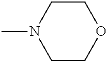 | 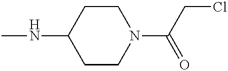 | OMe | 536.96 |
| 19 | VII | N | N | N | 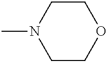 | 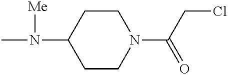 | OMe | 550.99 |
| 20 | VII | N | N | N | 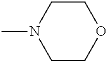 | 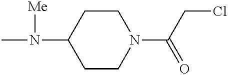 | O(CH₂)₃NMe₂ | 622.11 |
| 21 | VII | N | N | N | 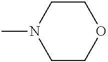 | 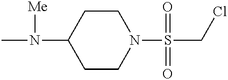 | OMe | 587.04 |
| 22 | VII | N | N | N | 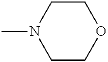 | 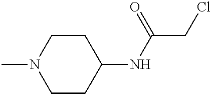 | OMe | 536.96 |
| 23 | VII | N | N | N | 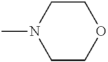 | 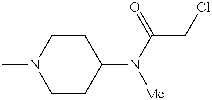 | OMe | 550.99 |
| 24 | VII | N | N | N | 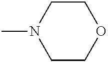 | 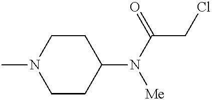 | O(CH₂)₃NMe₂ | 622.11 |
| 25 | VII | N | N | N | 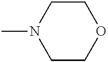 | 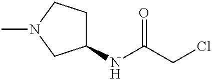 | OMe | 522.94 |
| 26 | VII | N | N | N | 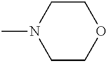 | 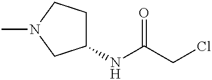 | OMe | 522.94 |
| 27 | VII | N | N | N | 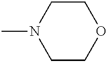 | 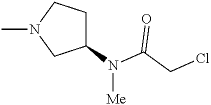 | OMe | 536.96 |
| 28 | VII | N | N | N | 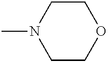 | 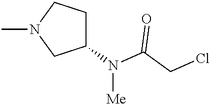 | OMe | 536.96 |
| 29 | VII | N | N | N | 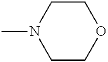 | 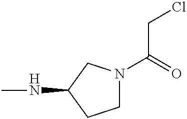 | OMe | 522.94 |
| 30 | VII | N | N | N | 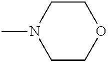 | 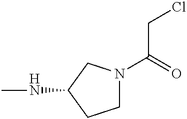 | OMe | 522.94 |
| 31 | VII | N | N | N | 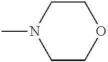 | 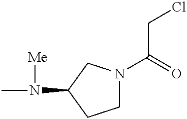 | OMe | 536.96 |
| 32 | VII | N | N | N | 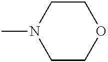 | 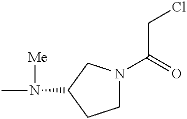 | OMe | 536.96 |
| 33 | VII | N | N | N | 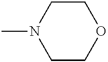 | 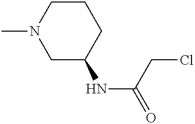 | OMe | 536.96 |
| 34 | VII | N | N | N | 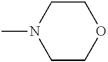 | 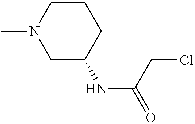 | OMe | 536.96 |
| 35 | VII | N | N | N | 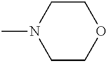 | 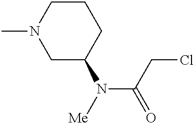 | OMe | 550.99 |
| 36 | VII | N | N | N | 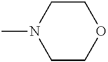 | 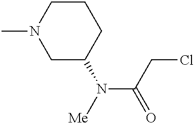 | OMe | 550.99 |
| 37 | VII | N | N | N | 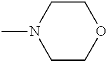 | 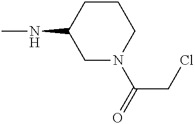 | OMe | 536.96 |
| 38 | VII | N | N | N | 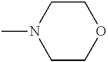 | 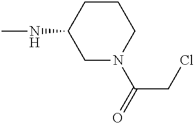 | OMe | 536.96 |
| 39 | VII | N | N | N | 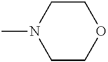 | 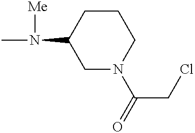 | OMe | 550.99 |
| 40 | VII | N | N | N | 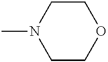 | 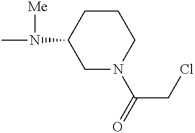 | OMe | 550.99 |
| 41 | VII | N | N | N | 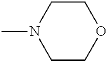 | 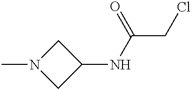 | OMe | 508.91 |
| 42 | VII | N | N | N | 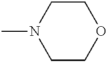 | 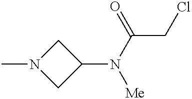 | OMe | 522.94 |
| 43 | VII | N | N | N | 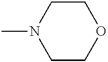 | 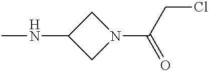 | OMe | 508.91 |
| 44 | VII | N | N | N | 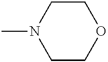 | 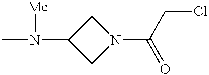 | OMe | 522.94 |
| 45 | VII | N | N | N | 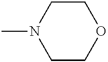 | 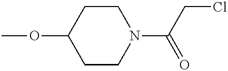 | OMe | 537.95 |
| 46 | VII | N | N | N | 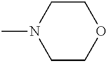 | 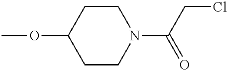 | O(CH₂)₃NMe₂ | 609.07 |
| 47 | VII | N | N | N | 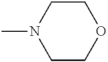 | 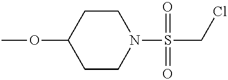 | O(CH₂)₃NMe₂ | 645.12 |
| 48 | XIV | CH | N | N | 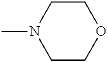 | 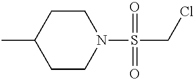 | OMe | 597.04 |

^{a}All compounds were analyzed by ¹H NMR and LCMS (APCI⁺), and had satisfactory combustion analyses for C, H, and N.

### Pharmaceutical Compositions

In one embodiment, provided herein is a pharmaceutical composition comprising a compound of Formula Ia, Ib, Ic, or Id as defined herein, and a pharmaceutically acceptable excipient, adjuvant, carrier, buffer, or stabiliser.

In one embodiment, the pharmaceutically acceptable excipient, adjuvant, carrier, buffer, or stabiliser is non-toxic and does not interfere with the efficacy of the active ingredient. The precise nature of the carrier or other material will depend on the route of administration, which may be oral or by injection, such as cutaneous, subcutaneous, or intravenous injection.

In one embodiment, the pharmaceutical compositions are provided in a dosage form for oral administration, which comprise a compound provided herein, and one or more pharmaceutically acceptable excipients or carriers. The pharmaceutical compositions provided herein that are formulated for oral administration may be in tablet, capsule, powder, or liquid form. A tablet may comprise a solid carrier or an adjuvant. Liquid pharmaceutical compositions generally comprise a liquid carrier such as water, petroleum, animal or vegetable oils, or mineral oil or synthetic oil. Physiological saline solution, dextrose or other saccharide solution, or glycols such as ethylene glycol, propylene glycol, or polyethylene glycol may be included. A capsule may comprise a solid carrier such as gelatin.

In another embodiment, the pharmaceutical compositions are provided in a dosage form for parenteral administration, and one or more pharmaceutically acceptable excipients or carriers. Where pharmaceutical compositions may be formulated for intravenous, cutaneous or subcutaneous injection, the active ingredient will be in the form of a parenterally acceptable aqueous solution, which is pyrogen-free and has a suitable pH, isotonicity, and stability. Those of relevant skill in the art are well able to prepare suitable solutions using, for example, isotonic vehicles, such as Sodium Chloride injection, Ringer's injection, or Lactated Ringer's injection. Preservatives, stabilisers, buffers, antioxidants, and/or other additives may be included as required.

In yet another embodiment, the pharmaceutical compositions are provided in a dosage form for topical administration, which comprise a compound provided herein, and one or more pharmaceutically acceptable excipients or carriers.

The pharmaceutical compositions can also be formulated as modified release dosage forms, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Delivery Technology, 2nd Edition, Rathbone et al., Eds., Marcel Dekker, Inc.: New York, N.Y., 2008).

The pharmaceutical compositions provided herein can be provided in a unit-dosage form or multiple-dosage form. A unit-dosage form, as used herein, refers to physically discrete a unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In another embodiment, the pharmaceutical compositions provided herein further comprise one or more chemotherapeutic agents as defined herein.

In yet another embodiment, provided herein is the use of a compound of Formula Ia, Ib, Ic, or Id in the manufacture of a medicament for the treatment of cancer. In certain embodiments, the medicament is in tablet, capsule, powder, or liquid form. In certain embodiments, the medicament is formulated as described herein.

### A. Oral Administration

The pharmaceutical compositions provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The amount of a binder or filler in the pharmaceutical compositions provided herein varies upon the type of formulation, and is readily discernible to those of ordinary skill in the art. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical compositions provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include, but are not limited to, colloidal silicon dioxide, CAB-O-SIL® (Cabot Co. of Boston, Mass.), and asbestos-free talc. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve several functions, even within the same formulation.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration can be also provided in the forms of liposomes, micelles, microspheres, or nanosystems. Micellar dosage forms can be prepared as described in U.S. Pat. No. 6,350,458.

The pharmaceutical compositions provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein for oral administration can be formulated as immediate or modified release dosage forms, including delayed-, sustained, pulsed-, controlled, targeted-, and programmed-release forms.

### B. Parenteral Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

When the pharmaceutical compositions provided herein are formulated for multiple dosage administration, the multiple dosage parenteral formulations must contain an antimicrobial agent at bacteriostatic or fungistatic concentrations. All parenteral formulations must be sterile, as known and practiced in the art.

In one embodiment, the pharmaceutical compositions for parenteral administration are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein for parenteral administration can be formulated as immediate or modified release dosage forms, including delayed-, sustained, pulsed-, controlled, targeted-, and programmed-release forms.

The pharmaceutical compositions provided herein for parenteral administration can be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the pharmaceutical compositions provided herein can also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions can also be administered topically by electroporation, iontophoresis, phonophoresis, sonophoresis, or microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellower wax, and appropriate mixtures of mono-, di- and triglycerides of fatty acids, and hydrogels, such as polyvinyl alcohol, hydroxyethyl methacrylate, and polyacrylic acid. Combinations of the various vehicles can also be used. Rectal and vaginal suppositories may be prepared by compressing or molding. The typical weight of a rectal and vaginal suppository is about 2 to about 3 g.

The pharmaceutical compositions provided herein can be administered ophthalmically in the forms of solutions, suspensions, ointments, emulsions, gel-forming solutions, powders for solutions, gels, ocular inserts, and implants.

The pharmaceutical compositions provided herein can be administered intranasally or by inhalation to the respiratory tract. The pharmaceutical compositions can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of the active ingredient provided herein; a propellant as solvent; and/or a surfactant, such as sorbitan trioleate, oleic acid, or an oligolactic acid.

The pharmaceutical compositions provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The pharmaceutical compositions provided herein for inhaled/intranasal administration can further comprise a suitable flavor, such as menthol and levomenthol; and/or sweeteners, such as saccharin and saccharin sodium.

The pharmaceutical compositions provided herein for topical administration can be formulated to be immediate release or modified release, including delayed-, sustained-, pulsed-, controlled-, targeted, and programmed release.

### D. Modified Release

The pharmaceutical compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

### 1. Matrix Controlled Release Devices

The pharmaceutical compositions provided herein in a modified release dosage form can be fabricated using a matrix controlled release device known to those skilled in the art (see, Takada et al. in “Encyclopedia of Controlled Drug Delivery,” Vol. 2, Mathiowitz Ed., Wiley, 1999).

In certain embodiments, the pharmaceutical compositions provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical compositions provided herein are formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions provided herein in a modified release dosage form can be prepared by methods known to those skilled in the art, including direct compression, dry or wet granulation followed by compression, and melt-granulation followed by compression.

### 2. Osmotic Controlled Release Devices

The pharmaceutical compositions provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein can be blended with the pharmaceutical compositions to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### 4. Targeted Delivery

The pharmaceutical compositions provided herein can also be formulated to be targeted to a particular tissue, receptor, or other area of the body of the subject to be treated, including liposome-, resealed erythrocyte-, and antibody-based delivery systems. Examples include, but are not limited to, those disclosed in U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### Methods of Use

In one embodiment, the compounds provided herein are designed to irreversibly inhibit the p110α isoform of PI3K, in one embodiment, to target the His-855 group which is considered to form part of the ATP binding pocket of the p110α isoform of PI3K, but not of the other isoforms. In targeting the His-855 group of p110α, the compounds, in one embodiment, selectively and irreversibly inhibit this PI3K isoform.

In one embodiment, provided herein is a method of irreversibly inhibiting PI3K. In certain embodiments, the compounds provided herein selectively target the His-855 moiety of the p110α subunit, thus selectively irreversibly inhibiting the p110α isoform of PI3K.

In one embodiment, provided herein is a method of cancer prevention or therapy for treating cancers, comprising administering a compound of Formula Ia, Ib, Ic, or Id as defined herein to a subject in need thereof.

In another embodiment, there is provided a method of cancer prevention or therapy for treating cancers, wherein the method comprises the steps of: 1) selectively targeting a p110α subunit of PI3K with a compound provided herein; and 2) alkylating the p110α subunit of PI3K with the compound. In certain embodiments, the compound provided herein selectively targets the p110α subunit of PI3K.

In yet another embodiment, there is provided a method of selectively irreversibly inhibiting the α isoform of PI3K, wherein the method comprises the steps of: 1) selectively targeting a p110α subunit of PI3K with a compound provided herein; and 2) alkylating the p110α subunit of PI3K with the compound. In certain embodiments, the compound provided herein selectively targets the p110α subunit of PI3K.

In one embodiment, provided is a method of treating, preventing, or ameliorating one or more symptoms of a disorder, disease, or condition associated with PI3K activity in a subject, which comprises administering to the subject a therapeutically effective amount of a compound provided herein, e.g., a compound of Formula Ia, Ib, Ic, or Id, including an enantiomer, a mixture of enantiomers, or a mixture of two or more diastereomers thereof; or a pharmaceutically acceptable salt, solvate, hydrate, or prodrug thereof.

In another embodiments, provided is a method of treating, preventing, or ameliorating one or more symptoms of a disorder, disease, or condition responsive to the modulation of PI3K activity in a subject, which comprises administering to the subject a therapeutically effective amount of a compound provided herein, e.g., a compound of Formula Ia, Ib, Ic, or Id, including an enantiomer, a mixture of enantiomers, or a mixture of two or more diastereomers thereof; or a pharmaceutically acceptable salt, solvate, hydrate, or prodrug thereof.

In yet another embodiment, provided is a method of treating, preventing, or ameliorating one or more symptoms of a disorder, disease, or condition mediated by a PI3K enzyme in a subject, which comprises administering to the subject a therapeutically effective amount of a compound provided herein, e.g., a compound of Formula Ia, Ib, Ic, or Id, including an enantiomer, a mixture of enantiomers, or a mixture of two or more diastereomers thereof; or a pharmaceutically acceptable salt, solvate, hydrate, or prodrug thereof.

In yet another embodiment, provided is a method of treating, preventing, or ameliorating one or more symptoms of cancer in a subject, which comprises administering to the subject a therapeutically effective amount of a compound provided herein, e.g., a compound of Formula Ia, Ib, Ic, or Id, including an enantiomer, a mixture of enantiomers, or a mixture of two or more diastereomers thereof; or a pharmaceutically acceptable salt, solvate, hydrate, or prodrug thereof.

In yet another embodiment, there is provided the use of a compound provided herein, e.g., a compound of Formula Ia, Ib, Ic, or Id, including an enantiomer, a mixture of enantiomers, or a mixture of two or more diastereomers thereof; or a pharmaceutically acceptable salt, solvate, hydrate, or prodrug thereof, in the manufacture of a medicament for the treatment of cancer. In certain embodiments, the compound selectively targets the p110α subunit of PI3K. In certain embodiments, the compound selectively inhibits the PI3K via its interaction with its p110α subunit. In certain embodiments, the compound selectively alkylates the p110α subunit of PI3K.

In one embodiment, the subject is a mammal. In another embodiment, the subject is a human. In yet another embodiment, the subject is a primate other than a human, a farm animal such as cattle, a sport animal, or a pet such as a horse, dog, or cat.

The disorders, diseases, or conditions treatable with a compound provided herein, include, but are not limited to, (1) inflammatory or allergic diseases, including systemic anaphylaxis and hypersensitivity disorders, atopic dermatitis, urticaria, drug allergies, insect sting allergies, food allergies (including celiac disease and the like), and mastocytosis; (2) inflammatory bowel diseases, including Crohn's disease, ulcerative colitis, ileitis, and enteritis; (3) vasculitis, and Behcet's syndrome; (4) psoriasis and inflammatory dermatoses, including dermatitis, eczema, atopic dermatitis, allergic contact dermatitis, urticaria, viral cutaneous pathologies including those derived from human papillomavirus, HIV or RLV infection, bacterial, flugal, and other parasital cutaneous pathologies, and cutaneous lupus erythematosus; (5) asthma and respiratory allergic diseases, including allergic asthma, exercise induced asthma, allergic rhinitis, otitis media, allergic conjunctivitis, hypersensitivity lung diseases, and chronic obstructive pulmonary disease; (6) autoimmune diseases, including arthritis (including rheumatoid and psoriatic), systemic lupus erythematosus, type I diabetes, myasthenia gravis, multiple sclerosis, Graves' disease, and glomerulonephritis; (7) graft rejection (including allograft rejection and graft-v-host disease), e.g., skin graft rejection, solid organ transplant rejection, bone marrow transplant rejection; (8) fever; (9) cardiovascular disorders, including acute heart failure, hypotension, hypertension, angina pectoris, myocardial infarction, cardiomyopathy, congestive heart failure, atherosclerosis, coronary artery disease, restenosis, and vascular stenosis; (10) cerebrovascular disorders, including traumatic brain injury, stroke, ischemic reperfusion injury and aneurysm; (11) cancers of the breast, skin, prostate, cervix, uterus, ovary, testes, bladder, lung, liver, larynx, oral cavity, colon and gastrointestinal tract (e.g., esophagus, stomach, pancreas), brain, thyroid, blood, and lymphatic system; (12) fibrosis, connective tissue disease, and sarcoidosis, (13) genital and reproductive conditions, including erectile dysfunction; (14) gastrointestinal disorders, including gastritis, ulcers, nausea, pancreatitis, and vomiting; (15) neurologic disorders, including Alzheimer's disease; (16) sleep disorders, including insomnia, narcolepsy, sleep apnea syndrome, and Pickwick Syndrome; (17) pain; (18) renal disorders; (19) ocular disorders, including glaucoma; and (20) infectious diseases, including HIV.

In certain embodiments, the cancer treatable with the methods provided herein includes, but is not limited to, (1) leukemias, including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome or a symptom thereof (such as anemia, thrombocytopenia, neutropenia, bicytopenia or pancytopenia), refractory anemia (RA), RA with ringed sideroblasts (RARS), RA with excess blasts (RAEB), RAEB in transformation (RAEB-T), preleukemia, and chronic myelomonocytic leukemia (CMML), (2) chronic leukemias, including, but not limited to, chronic myelocytic (granulocytic) leukemia, chronic lymphocytic leukemia, and hairy cell leukemia; (3) polycythemia vera; (4) lymphomas, including, but not limited to, Hodgkin's disease and non-Hodgkin's disease; (5) multiple myelomas, including, but not limited to, smoldering multiple myeloma, nonsecretory myeloma, osteosclerotic myeloma, plasma cell leukemia, solitary plasmacytoma, and extramedullary plasmacytoma; (6) Waldenström's macroglobulinemia; (7) monoclonal gammopathy of undetermined significance; (8) benign monoclonal gammopathy; (9) heavy chain disease; (10) bone and connective tissue sarcomas, including, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, metastatic cancers, neurilemmoma, rhabdomyosarcoma, and synovial sarcoma; (11) brain tumors, including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; (12) breast cancer, including, but not limited to, adenocarcinoma, lobular (small cell) carcinoma, intraductal carcinoma, medullary breast cancer, mucinous breast cancer, tubular breast cancer, papillary breast cancer, primary cancers, Paget's disease, and inflammatory breast cancer; (13) adrenal cancer, including, but not limited to, pheochromocytom and adrenocortical carcinoma; (14) thyroid cancer, including, but not limited to, papillary or follicular thyroid cancer, medullary thyroid cancer, and anaplastic thyroid cancer; (15) pancreatic cancer, including, but not limited to, insulinoma, gastrinoma, glucagonoma, vipoma, somatostatin-secreting tumor, and carcinoid or islet cell tumor; (16) pituitary cancer, including, but limited to, Cushing's disease, prolactin-secreting tumor, acromegaly, and diabetes insipius; (17) eye cancer, including, but not limited, to ocular melanoma such as iris melanoma, choroidal melanoma, and cilliary body melanoma, and retinoblastoma; (18) vaginal cancer, including, but not limited to, squamous cell carcinoma, adenocarcinoma, and melanoma; (19) vulvar cancer, including, but not limited to, squamous cell carcinoma, melanoma, adenocarcinoma, basal cell carcinoma, sarcoma, and Paget's disease; (20) cervical cancers, including, but not limited to, squamous cell carcinoma, and adenocarcinoma; (21) uterine cancer, including, but not limited to, endometrial carcinoma and uterine sarcoma; (22) ovarian cancer, including, but not limited to, ovarian epithelial carcinoma, borderline tumor, germ cell tumor, and stromal tumor; (23) esophageal cancer, including, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; (24) stomach cancer, including, but not limited to, adenocarcinoma, fungating (polypoid), ulcerating, superficial spreading, diffusely spreading, malignant lymphoma, liposarcoma, fibrosarcoma, and carcinosarcoma; (25) colon cancer; (26) rectal cancer; (27) liver cancer, including, but not limited to, hepatocellular carcinoma and hepatoblastoma; (28) gallbladder cancer, including, but not limited to, adenocarcinoma; (29) cholangiocarcinomas, including, but not limited to, pappillary, nodular, and diffuse; (30) lung cancer, including, but not limited to, non-small cell lung cancer, squamous cell carcinoma (epidermoid carcinoma), adenocarcinoma, large-cell carcinoma, and small-cell lung cancer; (31) testicular cancer, including, but not limited to, germinal tumor, seminoma, anaplastic, classic (typical), spermatocytic, nonseminoma, embryonal carcinoma, teratoma carcinoma, and choriocarcinoma (yolk-sac tumor); (32) prostate cancer, including, but not limited to, adenocarcinoma, leiomyosarcoma, and rhabdomyosarcoma; (33) penal cancer; (34) oral cancer, including, but not limited to, squamous cell carcinoma; (35) basal cancer; (36) salivary gland cancer, including, but not limited to, adenocarcinoma, mucoepidermoid carcinoma, and adenoidcystic carcinoma; (37) pharynx cancer, including, but not limited to, squamous cell cancer and verrucous; (38) skin cancer, including, but not limited to, basal cell carcinoma, squamous cell carcinoma and melanoma, superficial spreading melanoma, nodular melanoma, lentigo malignant melanoma, and acral lentiginous melanoma; (39) kidney cancer, including, but not limited to, renal cell cancer, adenocarcinoma, hypernephroma, fibrosarcoma, and transitional cell cancer (renal pelvis and/or uterer); (40) Wilms' tumor; (41) bladder cancer, including, but not limited to, transitional cell carcinoma, squamous cell cancer, adenocarcinoma, and carcinosarcoma; and other cancer, including, not limited to, myxosarcoma, osteogenic sarcoma, endotheliosarcoma, lymphangio-endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, and papillary adenocarcinomas (See Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Depending on the disorder, disease, or condition to be treated, and the subject's condition, the compounds or pharmaceutical compositions provided herein can be administered by oral, parenteral (e.g., intramuscular, intraperitoneal, intravenous, ICV, intracistemal injection or infusion, subcutaneous injection, or implant), inhalation, nasal, vaginal, rectal, sublingual, or topical (e.g., transdermal or local) routes of administration and can be formulated, alone or together, in suitable dosage unit with pharmaceutically acceptable excipients, carriers, adjuvants, and vehicles appropriate for each route of administration. Also provided is administration of the compounds or pharmaceutical compositions provided herein in a depot formulation, in which the active ingredient is released over a predefined time period.

In the treatment, prevention, or amelioration of one or more symptoms of the disorders, diseases, or conditions described herein, an appropriate dosage level generally is ranging from about 0.001 to 100 mg per kg subject body weight per day (mg/kg per day), from about 0.01 to about 75 mg/kg per day, from about 0.1 to about 50 mg/kg per day, from about 0.5 to about 25 mg/kg per day, or from about 1 to about 20 mg/kg per day, which can be administered in single or multiple doses. Within this range, the dosage can be ranging from about 0.005 to about 0.05, from about 0.05 to about 0.5, from about 0.5 to about 5.0, from about 1 to about 15, from about 1 to about 20, or from about 1 to about 50 mg/kg per day.

For oral administration, the pharmaceutical compositions provided herein can be formulated in the form of tablets containing from about 1.0 to about 1,000 mg of the active ingredient, in one embodiment, about 1, about 5, about 10, about 15, about 20, about 25, about 50, about 75, about 100, about 150, about 200, about 250, about 300, about 400, about 500, about 600, about 750, about 800, about 900, and about 1,000 mg of the active ingredient for the symptomatic adjustment of the dosage to the patient to be treated. The pharmaceutical compositions can be administered on a regimen of 1 to 4 times per day, including once, twice, three times, and four times per day.

It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Also provided herein are methods of modulating PI3K activity, comprising contacting a PIK3 enzyme with a compound provided herein, e.g., a compound of Formula Ia, Ib, Ic, or Id, including an enantiomer, a mixture of enantiomers, or a mixture of two or more diastereomers thereof; or a pharmaceutically acceptable salt, solvate, hydrate, or prodrug thereof. In one embodiment, the PIK3 enzyme is inside a cell.

The compounds provided herein, e.g., a compound of Formula Ia, Ib, Ic, or Id, including an enantiomer, a mixture of enantiomers, or a mixture of two or more diastereomers thereof; or a pharmaceutically acceptable salt, solvate, hydrate, or prodrug thereof, can also be combined or used in combination with other agents or therapies useful in the treatment, prevention, or amelioration of one or more symptoms of the disorders, diseases, or conditions for which the compounds provided herein are useful, including asthma, allergic rhinitis, eczema, psoriasis, atopic dermatitis, fever, sepsis, systemic lupus erythematosus, diabetes, rheumatoid arthritis, multiple sclerosis, atherosclerosis, transplant rejection, inflammatory bowel disease, cancer, infectious diseases, and those pathologies noted herein.

Suitable other therapeutic agents can also include, but are not limited to, (1) alpha-adrenergic agents; (2) antiarrhythmic agents; (3) anti-atherosclerotic agents, such as ACAT inhibitors; (4) antibiotics, such as anthracyclines, bleomycins, mitomycin, dactinomycin, and plicamycin; (5) anticancer agents and cytotoxic agents, e.g., alkylating agents, such as nitrogen mustards, alkyl sulfonates, nitrosoureas, ethylenimines, and triazenes; (6) anticoagulants, such as acenocoumarol, argatroban, bivalirudin, lepirudin, fondaparinux, heparin, phenindione, warfarin, and ximelagatran; (7) anti-diabetic agents, such as biguanides (e.g., metformin), glucosidase inhibitors (e.g., acarbose), insulins, meglitinides (e.g., repaglinide), sulfonylureas (e.g., glimepiride, glyburide, and glipizide), thiozolidinediones (e.g., troglitazone, rosiglitazone, and pioglitazone), and PPAR-gamma agonists; (8) antifungal agents, such as amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole; (9) antiinflammatories, e.g., non-steroidal anti-inflammatory agents, such as aceclofenac, acemetacin, amoxiprin, aspirin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, and tolmetin; (10) antimetabolites, such as folate antagonists, purine analogues, and pyrimidine analogues; (11) anti-platelet agents, such as GPIIb/IIIa blockers (e.g., abciximab, eptifibatide, and tirofiban), P2Y(AC) antagonists (e.g., clopidogrel, ticlopidine and CS-747), cilostazol, dipyridamole, and aspirin; (12) antiproliferatives, such as methotrexate, FK506 (tacrolimus), and mycophenolate mofetil; (13) anti-TNF antibodies or soluble TNF receptor, such as etanercept, rapamycin, and leflunimide; (14) aP2 inhibitors; (15) beta-adrenergic agents, such as carvedilol and metoprolol; (16) bile acid sequestrants, such as questran; (17) calcium channel blockers, such as amlodipine besylate; (18) chemotherapeutic agents; (19) cyclooxygenase-2 (COX-2) inhibitors, such as celecoxib and rofecoxib; (20) cyclosporins; (21) cytotoxic drugs, such as azathioprine and cyclophosphamide; (22) diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, ticrynafen, chlorthalidone, furosenide, muzolimine, bumetanide, triamterene, amiloride, and spironolactone; (23) endothelin converting enzyme (ECE) inhibitors, such as phosphoramidon; (24) enzymes, such as L-asparaginase; (25) Factor VIIa Inhibitors and Factor Xa Inhibitors; (26) farnesyl-protein transferase inhibitors; (27) fibrates; (28) growth factor inhibitors, such as modulators of PDGF activity; (29) growth hormone secretagogues; (30) HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, atavastatin, or visastatin); neutral endopeptidase (NEP) inhibitors; (31) hormonal agents, such as glucocorticoids (e.g., cortisone), estrogens/antiestrogens, androgens/antiandrogens, progestins, and luteinizing hormone-releasing hormone antagonists, and octreotide acetate; (32) immunosuppressants; (33) mineralocorticoid receptor antagonists, such as spironolactone and eplerenone; (34) microtubule-disruptor agents, such as ecteinascidins; (35) microtubule-stabilizing agents, such as pacitaxel, docetaxel, and epothilones A-F; (36) MTP Inhibitors; (37) niacin; (38) phosphodiesterase inhibitors, such as PDE III inhibitors (e.g., cilostazol) and PDE V inhibitors (e.g., sildenafil, tadalafil, and vardenafil); (39) plant-derived products, such as vinca alkaloids, epipodophyllotoxins, and taxanes; (40) platelet activating factor (PAF) antagonists; (41) platinum coordination complexes, such as cisplatin, satraplatin, and carboplatin; (42) potassium channel openers; (43) prenyl-protein transferase inhibitors; (44) protein tyrosine kinase inhibitors; (45) renin inhibitors; (46) squalene synthetase inhibitors; (47) steroids, such as aldosterone, beclometasone, betamethasone, deoxycorticosterone acetate, fludrocortisone, hydrocortisone (cortisol), prednisolone, prednisone, methylprednisolone, dexamethasone, and triamcinolone; (48) TNF-alpha inhibitors, such as tenidap; (49) thrombin inhibitors, such as hirudin; (50) thrombolytic agents, such as anistreplase, reteplase, tenecteplase, tissue plasminogen activator (tPA), recombinant tPA, streptokinase, urokinase, prourokinase, and anisoylated plasminogen streptokinase activator complex (APSAC); (51) thromboxane receptor antagonists, such as ifetroban; (52) topoisomerase inhibitors; (53) vasopeptidase inhibitors (dual NEP-ACE inhibitors), such as omapatrilat and gemopatrilat; and (54) other miscellaneous agents, such as, hydroxyurea, procarbazine, mitotane, hexamethylmelamine, and gold compounds.

In certain embodiments, the other therapies that may be used in combination with the compounds provided herein include, but are not limited to, surgery, endocrine therapy, biologic response modifiers (e.g., interferons, interleukins, and tumor necrosis factor (TNF)), hyperthermia and cryotherapy, and agents to attenuate any adverse effects (e.g., antiemetics).

In certain embodiments, the other therapeutic agents that may be used in combination with the compounds provided herein include, but are not limited to, alkylating drugs (mechlorethamine, chlorambucil, cyclophosphamide, melphalan, and ifosfamide), antimetabolites (cytarabine (also known as cytosine arabinoside or Ara-C), HDAC (high dose cytarabine), and methotrexate), purine antagonists and pyrimidine antagonists (6-mercaptopurine, 5-fluorouracil, cytarbine, and gemcitabine), spindle poisons (vinblastine, vincristine, and vinorelbine), podophyllotoxins (etoposide, irinotecan, and topotecan), antibiotics (daunorubicin, doxorubicin, bleomycin, and mitomycin), nitrosoureas (carmustine and lomustine), enzymes (asparaginase), and hormones (tamoxifen, leuprolide, flutamide, and megestrol), imatinib, adriamycin, dexamethasone, and cyclophosphamide. For a more comprehensive discussion of updated cancer therapies; See, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

In another embodiment, the method provided herein comprises administration of a compound of Formula Ia, Ib, Ic, or Id together with administering one or more chemotherapeutic agents and/or therapies selected from: alkylation agents (e.g., cisplatin, carboplatin); antimetabolites (e.g., methotrexate and 5-FU); antitumour antibiotics (e.g., adriamymycin and bleomycin); antitumour vegetable alkaloids (e.g., taxol and etoposide); antitumor hormones (e.g., dexamethasone and tamoxifen); antitumour immunological agents (e.g., interferon α, β, and γ); radiation therapy; and surgery. In certain embodiments, the one or more chemotherapeutic agents and/or therapies are administered to the subject before, during, or after the administration of the compound of Formula Ia, Ib, Ic, or Id as defined herein.

Such other agents, or drugs, can be administered, by a route and in an amount commonly used therefor, simultaneously or sequentially with the compounds provided herein, e.g., a compound of Formula I, including a single enantiomer, a mixture of enantiomers, or a mixture of diastereomers thereof; or a pharmaceutically acceptable salt, solvate, or prodrug thereof. When a compound provided herein is used contemporaneously with one or more other drugs, a pharmaceutical composition containing such other drugs in addition to the compound provided herein can be utilized, but is not required. Accordingly, the pharmaceutical compositions provided herein include those that also contain one or more other active ingredients or therapeutic agents, in addition to a compound provided herein.

The weight ratio of a compound provided herein to the second active ingredient can be varied, and will depend upon the effective dose of each ingredient. Generally, an effective dose of each will be used. Thus, for example, when a compound provided herein is combined with a NSAID, the weight ratio of the compound to the NSAID can range from about 1,000:1 to about 1:1,000, or about 200:1 to about 1:200. Combinations of a compound provided herein and other active ingredients will generally also be within the aforementioned range, but in each case, an effective dose of each active ingredient should be used.

The compounds provided herein can also be provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

Provided herein also are kits which, when used by the medical practitioner, can simplify the administration of appropriate amounts of active ingredients to a subject. In certain embodiments, the kit provided herein includes a container and a dosage form of a compound provided herein, including a single enantiomer or a mixture of diastereomers thereof; or a pharmaceutically acceptable salt, solvate, or prodrug thereof.

In certain embodiments, the kit includes a container comprising a dosage form of the compound provided herein, including a single enantiomer or a mixture of diastereomers thereof; or a pharmaceutically acceptable salt, solvate, or prodrug thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); M (molar); mM (millimolar); μM (micromolar); eq. (equivalent); Hz (Hertz); MHz (megahertz); mmol (millimoles); hr or hrs (hours); min (minutes); conc. (concentrated); mp (melting point); MS (mass spectrometry); ESI (electrospray ionization); TLC (thin layer chromatography); DMF (dimethylormamide); DMSO (dimethylsulfoxide); DMSO-d₆ (deuterated dimethylsulfoxide); EtOH (ethanol); EtOAc (ethyl acetate); i-PrOH (isopropanol); MeOH (methanol); THF (tetrahydrofuran); DIPEA (N,N-diisopropylethylamine); TFA (triethylacetic acid); TBDMSCl (tert-butylchlorodimethylsilane); TBAF, (tetra-n-butylammonium fluoride); PdCl₂(dppf), ((1,1′-bis(diphenylphosphino)ferrocene) dichloropalladium(II)); EDTA, (ethylenediaminetetraacetic acid); Me (methyl); Et (ethyl); tBu (tert-butyl); and Boc (tert-butoxylcarbony).

For all of the following examples, standard work-up and purification methods known to those skilled in the art can be utilized. Unless otherwise indicated, all temperatures are expressed in ° C. (degrees Centigrade). All reactions conducted at room temperature unless otherwise noted. Synthetic methodologies illustrated herein are intended to exemplify the applicable chemistry through the use of specific examples and are not indicative of the scope of the disclosure.

### General Experimental Information.

The following examples are representative of the disclosure, and provide detailed methods for preparing the compounds of the disclosure, including the preparation of intermediate compounds. In these examples, elemental analyses (combustion analysis) were carried out in the Microchemical Laboratory, University of Otago, Dunedin, NZ. Melting points were determined on an Electrothermal 9100 Melting Point Apparatus. NMR spectra were obtained on a Bruker Avance-400 spectrometer at 400 MHz for ¹H and 100 MHz for ¹³C spectra, referenced to TMS (Si(CH₃)₄). Mass spectra were determined on a VG-70SE mass spectrometer using an ionizing potential of 70 eV at a nominal resolution of 1000. High-resolution spectra were obtained at nominal resolutions of 3000, 5000, or 10000 as appropriate. All spectra were obtained as electron impact (EI) using Perfluorokerosene (PFK) as the reference unless otherwise stated. Column chromatography was carried out on silica gel (Merck 230-400 mesh), unless otherwise stated.

### Example 1

### Synthesis of 3-[4-[4-(chloroacetyl)-1-piperazinyl]-6-(4-morpholinyl)-1,3,5-triazin-2-yl]phenol

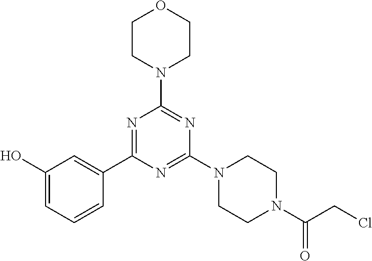

The compound was prepared according to Scheme 1 using Method A.

A mixture of 2.35 g (10 mmol) of 2,4-dichloro-6-(4-morpholinyl)-1,3,5-triazine, 1.93 g (10.5 mmol) of tert-butyl 1-piperazinecarboxylate (1-Boc-piperazine), and 1.38 g (10 mmol) powdered K₂CO₃ in 20 mL acetone was stirred at room temperature for 30 min before being diluted with water to give 3.70 g (96% yield) of tert-butyl 4-[4-chloro-6-(4-morpholinyl)-1,3,5-triazin-2-yl]-1-piperazinecarboxylate as a white solid: mp (CH₂Cl₂/MeOH) 178-180° C.; ¹H NMR (CDCl₃) δ 3.78 (m, 8H), 3.71 (m, 4H), 3.46 (m, 4H), 1.48 (s, 9H); MS (APCI⁺) 385.8 (MH⁺); Anal. Calcd. for C₁₆H₂₅ClN₆O₃: C, 49.9; H, 6.55; N, 21.8. Found: C, 50.15; H, 6.5; N, 22.1%.

A mixture of 0.385 g (1 mmol) of tert-butyl 4-[4-chloroa-6-(4-morpholinyl)-1,3,5-triazin-2-yl]-1-piperazinecarboxylate, 0.33 g (1.5 mmol) of 3-(4,4,5,5-tetramethyl-1,3,2-dioxaborolan-2-yl)phenol, 8 mL of 2 M Na₂CO₃, and 56 mg (0.08 mmol) of PdCl₂(dppf) in 40 mL dioxane was refluxed under an atmosphere of nitrogen for 3 hrs. After cooling, the dioxane was removed under vacuum and the residue was extracted with CH₂Cl₂. After drying, the solvent was removed and the product was purified by chromatography on silica gel, eluting with CH₂Cl₂/EtOAc (4:1), to give 0.39 g (88% yield) of tert-butyl 4-[4-(3-hydroxyphenyl)-6-(4-morpholinyl)-1,3,5-triazin-2-yl]piperazine-1-carboxylate: mp (MeOH) 180-182° C.; ¹H NMR (CDCl₃) δ 7.95 (d, J=7.9 Hz, 1H), 7.29 (m, 1H), 7.30 (t, J=7.9 Hz, 1H), 6.96 (dd, J=8.0, 2.7 Hz, 1H), 5.36 (m, exchangeable with D₂O, 1H), 3.90 (m, 8H), 3.76 (m, 4H), 3.59 (m, 4H), 1.50 (s, 9H); MS (APCI⁺) 444.1 (MH⁺); Anal. Calcd. for C₂₂H₃₀N₆O₄: C, 59.71; H, 6.83; N, 18.99. Found: C, 59.6; H, 7.0; N, 19.0%.

A mixture of 0.32 g (0.723 mmol) of tert-butyl 4-[4-(3-hydroxyphenyl)-6-(4-morpholinyl)-1,3,5-triazin-2-yl]piperazine-1-carboxylate and 0.41 g (3.6 mmol) of trifluoroacetic acid in 5 mL of CH₂Cl₂ was stirred at room temperature for 1 hr, before the solution was diluted with further CH₂Cl₂ and washed with 2 M aqueous ammonia. The organic layer was then dried and removed under vacuum to give 0.246 g (99% yield) of 3-[4-(4-morpholinyl)-6-(1-piperazinyl)-1,3,5-triazin-2-yl]phenol: mp (MeOH/1-PrOH) 273-277° C.; ¹H NMR (DMSO-d₆) δ 9.47 (s, exchangeable with D₂O, 1H), 7.76 (m, 2H), 7.25 (t, J=8.1 Hz, 1H), 6.90 (m, 1H), 3.79 (m, 8H), 3.65 (m, 4H), 2.73 (m, 4H); MS (APCI⁺) 343.7 (MH⁺); Anal. Calcd. For C₁₇H₂₂N₆O₂: C, 59.6; H, 6.5; N, 24.5. Found: C, 59.6; H, 6.4; N, 24.65%.

A stirred mixture of 103 mg (0.3 mmol) of 3-[4-(4-morpholinyl)-6-(1-piperazinyl)-1,3,5-triazin-2-yl]phenol and 120 mg (0.9 mmol) of N-ethyl-N,N-diisopropylamine in 100 mL of CH₂Cl₂ was cooled to 0° C. and treated dropwise with 85 mg (0.7 mmol) of 2-chloroacetyl chloride. The mixture was allowed to warm to room temperature, and was then washed successively with dilute aqueous acetic acid and aqueous NaHCO₃ solution. The organic solvent was removed under vacuum, and the residue was dissolved in a mixture of acetone and aqueous NaHCO₃ solution. After TLC indicated complete hydrolysis of the initially formed O-(2-chloroacetate), the acetone was removed under vacuum and the residue was extracted with CH₂Cl₂. After drying, the solvent was removed to give 100 mg (80% yield) of 3-[4-[4-(chloroacetyl)-1-piperazinyl]-6-(4-morpholinyl)-1,3,5-triazin-2-yl]phenol: mp (MeOH) 242-245° C.; ¹H NMR (DMSO-d₆) δ 9.51 (s, exchangeable with D₂O, 1H), 7.78 (m, 2H), 7.26 (t, J=7.8 Hz, 1H), 6.92 (m, 1H), 4.44 (s, 2H), 3.82 (m, 8H), 3.38 (m, 4H), 3.57 (m, 4H); MS (APCI⁺) 419.9 (MH⁺); Anal. Calcd. for C₁₉H₂₃ClN₆O₃: C, 54.5; H, 5.5; N, 20.1. Found: C, 54.4; H, 5.6; N, 19.8%.

### Example 2

### Synthesis of 1-[4-[4-(chloroacetyl)-1-piperazinyl]-6-(4-morpholinyl)-1,3,5-triazin-2-yl]-2-(difluoromethyl)-4-ethoxy-1H-benzimidazole

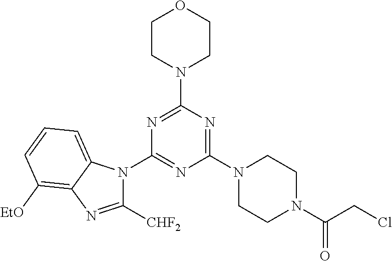

The compound was prepared according to Scheme 2 using Method B.

Treatment of tert-butyl 4-(chloroacetyl)piperazine-1-carboxylate (An et al., Tetrahedron, 1998, 54, 3999) with excess trifluoroacetic acid in CH₂Cl₂ at room temperature, followed by removal of the solvent under vacuum gave crude 4-(chloroacetyl)-1-piperazinium trifluoroacetate: ¹H NMR (DMSO-d₆) δ 8.94 (br s, exchangeable with D₂O, 2H), 4.44 (s, 2H), 3.66 (m, 4H), 3.13 (m, 4H), MS (APCI⁺) 163.5 (MH⁺).

A solution of 1.64 g (9 mmol) of 2-ethoxy-6-nitroaniline in methanol was hydrogenated over palladium on carbon. After filtration through celite, the solution was acidified with conc. HCl and evaporated to dryness. The residue was combined with 1.73 g (18 mmol) of difluoroacetic acid in 10 mL 4 M HCl and the solution was heated under reflux for 4 hrs. After dilution with water, decolorization with charcoal, and filtration through celite, the cooled solution was made basic with conc. aqueous ammonia to give 1.29 g (68% yield) of 2-(difluoromethyl)-4-ethoxy-1H-benzimidazole: mp (MeOH/H₂O) 185-187° C.; ¹H NMR (DMSO-d₆) (tautomeric mixture) δ 13.30 (m, exchangeable with D₂O, 1H), 7.20 (t, J_{HF}=53.3 Hz, 1H), 7.19 (m, 2H), 6.78 (br d, J=7.5 Hz, 1H), 4.24 (q, J=7.0 Hz, 2H), 1.41 (t, J=7.0 Hz, 3H); MS (APCI⁺) 213.3 (MH⁺); Anal. Calcd. for C₁₀H₁₀F₂N₂O: C, 56.6; H, 4.75; N, 13.2. Found: C, 56.9; H, 4.8; N, 13.4%.

A mixture of 0.85 g (4 mmol) of 2-(difluoromethyl)-4-ethoxy-1H-benzimidazole, 0.94 g (4 mmol) of 2,4-dichloro-6-(4-morpholinyl)-1,3,5-triazine, and 4.4 g (32 mmol) of powdered K₂CO₃ in 25 mL DMF was stirred at room temperature overnight. The mixture was diluted with water and the precipitate was collected and washed successively with water and cold ethanol to give 1.48 g (90% yield) of 1-[4-chloro-6-(4-morpholinyl)-1,3,5-triazin-2-yl]-2-(difluoromethyl)-4-ethoxy-1H-benzimidazole: mp (EtOH) 272-275° C.; ¹H NMR (CDCl₃) δ 7.98 (d, J=8.4 Hz, 1H), 7.47 (t, J_{HF}=53.4 Hz, 1H), 7.38 (t, J=8.3 Hz, 1H), 6.86 (d, J=8.1 Hz, 1H), 4.33 (q, J=7.0 Hz, 2H), 3.96 (m, 4H), 3.81 (m, 4H), 1.56 (s, 3H); MS (APCI⁺) 411.7/413.7 (MH⁺); Anal. Calcd. for C₁₇H₁₇ClF₂N₆O₂: C, 49.7; H, 4.2; N, 20.5. Found: C, 49.8; H, 4.4; N, 20.6%.

A suspension of 103 mg (0.25 mmol) of 1-[4-chloro-6-(4-morpholinyl)-1,3,5-triazin-2-yl]-2-(difluoromethyl)-4-ethoxy-1H-benzimidazole and 200 mg (0.75 mmol) of 4-(chloroacetyl)-1-piperazinium trifluoroacetate in 20 mL of THF was treated with 0.16 g (1.25 mmol) of N-ethyl-N,N-diisopropylamine and the mixture was stirred at room temperature for 3 hrs. The resulting clear solution was diluted with water to give 127 mg (94% yield) of 1-[4-[4-(chloro acetyl)-1-piperazinyl]-6-(4-morpholinyl)-1,3,5-triazin-2-yl]-2-(difluoromethyl)-4-ethoxy-M-benzimidazole: mp (CH₂Cl₂/EtOH) 217-219° C.; ¹H NMR (CDCl₃) δ 7.86 (d, J=8.4 Hz, 1H), 7.44 (t, J_{HF}=53.5 Hz, 1H), 7.33 (t, J=8.3 Hz, 1H), 6.82 (d, J=7.9 Hz, 1H), 4.33 (q, J=7.0 Hz, 2H), 4.13 (s, 2H), 3.99 (m, 2H), 3.89 (m, 6H), 3.79 (m, 4H), 3.74 (m, 2H), 3.64 (m, 2H), 1.56 (t, J=7.0 Hz, 3H); MS (APCI⁺) 538.3/540.3 (MH⁺); Anal. Calcd. for C₂₃H₂₇ClF₂N₈O₃: C, 51.45; H, 5.1; N, 20.9. Found: C, 51.75; H, 5.3; N, 21.0%.

### Example 3

### Synthesis of 1-[4-[4-(Chloroacetyl)-1-piperazinyl]-6-(4-morpholinyl)-1,3,5-triazin-2-yl]-2-(difluoromethyl)-1H-benzimidazole

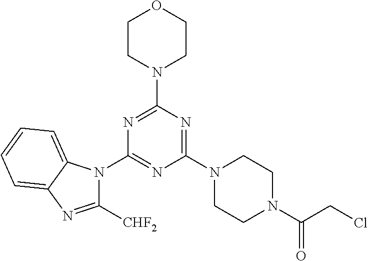

The compound was prepared according to Scheme 2 using Method B.

Reaction of 2-(difluoromethyl)-1H-benzimidazole with of 2,4-dichloro-6-(4-morpholinyl)-1,3,5-triazine as in Example 2 gave 1-[4-chloro-6-(4-morpholinyl)-1,3,5-triazin-2-yl]-2-(difluoromethyl)-1H-benzimidazole (U.S. Pat. Appl. No. 2007/244110): mp (CHCl₃/EtOH) 249-252° C.; ¹H NMR (CDCl₃) δ 8.42 (d, J=7.4 Hz, 1H), 7.90 (d, J=7.4 Hz, 1H), 7.57 (t, J_{HF}=53.5 Hz, 1H), 7.50 (m, 2H), 3.98 (m, 4H), 3.83 (m, 4H); MS (APCI⁺) 367.2/369.2 (MH⁺); Anal. Calcd. for C₁₅H₁₃ClF₂N₆O: C, 49.1; H, 3.6; N, 22.9. Found: C, 49.3; H, 3.5; N, 22.9%.

Reaction of 1-[4-chloro-6-(4-morpholinyl)-1,3,5-triazin-2-yl]-2-(difluoromethyl)-1H-benzimidazole with 4-(chloroacetyl)-1-piperazinium trifluoroacetate as in Example 2 gave 1-[4-[4-(chloroacetyl)-1-piperazinyl]-6-(4-morpholinyl)-1,3,5-triazin-2-yl]-2-(difluoromethyl)-1H-benzimidazole: mp (i-PrOH) 189-191° C.; ¹H NMR (DMSO-d₆) δ 8.35 (d, J=8.1 Hz, 1H), 7.85 (d, J=7.9 Hz, 1H), 7.75 (t, J_{HF}=52.7 Hz, 1H), 7.51 (t, J=7.7 Hz, 1H), 7.44 (t, J=7.6 Hz, 1H), 4.46 (s, 2H), 3.89 (m, 2H), 3.83 (m, 6H), 3.70 (m, 4H), 3.61 (m, 4H); MS (APCI⁺) 493.8/495.8 (MH⁺); Anal. Calcd. for C₂₁H₂₄Cl₂F₂N₈O₂: C, 51.2; H, 4.7; N, 22.7; Cl, 7.2. Found: C, 51.1; H, 4.65; N, 22.4; Cl, 7.5%.

### Example 4

### Synthesis of 1-[4-[4-(Chloroacetyl)-1-piperazinyl]-6-(4-morpholinyl)-1,3,5-triazin-2-yl]-2-(difluoromethyl)-4-methoxy-1H-benzimidazole

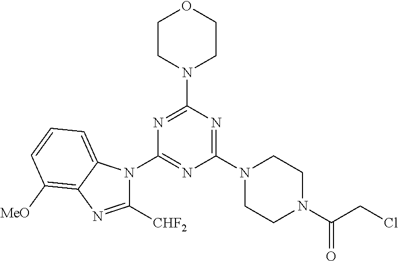

The compound was prepared according to Scheme 2 using Method B.

Reaction of 2-(difluoromethyl)-4-methoxy-1H-benzimidazole with of 2,4-dichloro-6-(4-morpholinyl)-1,3,5-triazine as in Example 2 gave 1-[4-chloro-6-(4-morpholinyl)-1,3,5-triazin-2-yl]-2-(difluoromethyl)-4-methoxy-1H-benzimidazole: mp (CHCl₃/EtOH) 263-266° C.; ¹H NMR (CDCl₃) δ 7.99 (d, J=8.4 Hz, 1H), 7.48 (t, J_{HF}=53.4 Hz, 1H), 7.40 (t, J=8.3 Hz, 1H), 6.86 (d, J=8.1 Hz, 1H), 4.05 (s, 3H), 3.96 (m, 4H), 3.82 (m, 4H); MS (APCI⁺) 397.8/399.8 (MH⁺); Anal. Calcd. for C₁₆H₁₅ClF₂N₆O₂: C, 48.4; H, 3.8; N, 21.2. Found: C, 48.3; H, 3.8; N, 21.1%.

Reaction of 1-[4-chloro-6-(4-morpholinyl)-1,3,5-triazin-2-yl]-2-(difluoromethyl)-4-methoxy-1H-benzimidazole with 4-(chloroacetyl)-1-piperazinium trifluoroacetate as in Example 2 gave 1-[4-[4-(chloroacetyl)-1-piperazinyl]-6-(4-morpholinyl)-1,3,5-triazin-2-yl]-2-(difluoromethyl)-4-methoxy-1H-benzimidazole: mp (MeOH) 241-243° C.; ¹H NMR (CDCl₃) δ 7.87 (d, J=8.4 Hz, 1H), 7.45 (t, J_{HF}=53.5 Hz, 1H), 7.36 (t, J=8.2 Hz, 1H), 6.82 (d, J=7.8 Hz, 1H), 4.13 (s, 2H), 4.05 (s, 3H), 3.99 (m, 2H), 3.89 (m, 6H), 3.79 (m, 4H), 3.74 (m, 2H), 3.65 (m, 2H); MS (APCI⁺) 524.0/526.0 (MH⁺); Anal. Calcd. for C₂₂H₂₅ClF₂N₈O₃: C, 50.5; H, 4.8; N, 21.4; Cl, 6.8. Found: C, 50.7; H, 4.8; N, 21.4; Cl, 6.9%.

### Example 5

### Synthesis of 1-[4-[4-(Chloroacetyl)-1-piperazinyl]-6-(4-morpholinyl)-1,3,5-triazin-2-yl]-2-(difluoromethyl)-1H-benzimidazol-4-ol

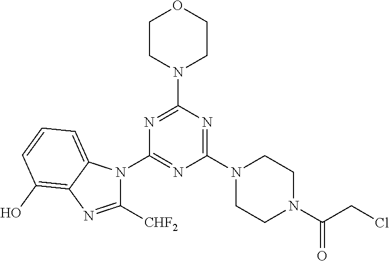

The compound was prepared according to Scheme 1 using Method A.

Reaction of 4-(tert-butyldimethylsilyloxy)-2-(difluoromethyl)-1H-benzimidazole with 2,4-dichloro-6-(4-morpholinyl)-1,3,5-triazine as in Example 2, but using acetone as solvent, followed by chromatography on silica gel (eluting with CH₂Cl₂/hexanes (3:1)) gave 4-(tert-butyldimethylsilyloxy)-1-[4-chloro-6-(4-morpholinyl)-1,3,5-triazin-2-yl]-2-(difluoromethyl)-1H-benzimidazole: mp (hexanes) 143-145° C.; ¹H NMR (CDCl₃) δ 7.99 (d, J=8.4 Hz, 1H), 7.46 (t, J_{HF}=53.5 Hz, 1H), 7.32 (t, J=8.2 Hz, 1H), 6.88 (d, J=8.0 Hz, 1H), 3.96 (m, 3.5H), 3.88 (m, 0.5H), 3.81 (m, 3.5H), 3.75 (m, 0.5H), 1.05 (s, 9H), 0.29 (s, 6H); MS (APCI⁺) 497.9/499.9 (MH⁺); Anal. Calcd. for C₂₁H₂₇ClF₂N₆O₂Si: C, 50.75; H, 5.5; N, 16.9. Found: C, 50.7; H, 5.6; N, 17.0%.

Reaction of 4-(tert-butyldimethylsilyloxy)-1-[4-chloro-6-(4-morpholinyl)-1,3,5-triazin-2-yl]-2-(difluoromethyl)-1H-benzimidazole with 2.2 equivalents of tert-butyl 1-piperazinecarboxylate in THF at room temperature gave a quantitative yield of tert-butyl 4-[4-[4-(tert-butyldimethylsilyloxy)-2-(difluoromethyl)-1H-benzimidazol-1-yl]-6-(4-morpholinyl)-1,3,5-triazin-2-yl]piperazine-1-carboxylate as an oil: ¹H NMR (CDCl₃) δ 7.91 (d, J=8.2 Hz, 1H), 7.45 (t, J_{HF}=53.6 Hz, 1H), 7.26 (t, J=8.1 Hz, 1H), 6.83 (d, J=7.9 Hz, 1H), 3.85 (m, 8H), 3.77 (m, 4H), 3.53 (m, 4H), 1.50 (s, 9H), 1.05 (s, 9H), 0.30 (s, 6H); MS (APCI⁺) 648.7 (MH⁺).

Reaction of crude tert-butyl 4-[4-[4-(tert-butyldimethylsilyloxy)-2-(difluoromethyl)-1H-benzimidazol-1-yl]-6-(4-morpholinyl)-1,3,5-triazin-2-yl]piperazine-1-carboxylate with tetrabutylammonium fluoride in THF at 0° C. gave a quantitative yield of tert-butyl 4-[4-[2-(difluoromethyl)-4-hydroxy-1H-benzimidazol-1-yl]-6-(4-morpholinyl)-1,3,5-triazin-2-yl]piperazine-1-carboxylate: mp (MeOH) 228-230° C.; ¹H NMR (CDCl₃) δ 7.81 (d, J=8.4 Hz, 1H), 7.55 (t, J_{HF}=53.6 Hz, 1H), 7.32 (t, J=8.2 Hz, 1H), 6.90 (d, J=8.0 Hz, 1H), 3.88 (m, 8H), 3.79 (m, 4H), 3.53 (m, 4H), 1.50 (s, 9H); MS (APCI⁺) 534.1 (MH⁺); Anal. Calcd. for C₂₄H₃₀F₂N₈O₄: C, 54.1; H, 5.7; N, 21.0. Found: C, 54.15; H, 5.8; N, 21.3%.

Reaction of tert-butyl 4-[4-[2-(difluoromethyl)-4-hydroxy-1H-benzimidazol-1-yl]-6-(4-morpholinyl)-1,3,5-triazin-2-yl]piperazine-1-carboxylate with 5 equivalents of trifluoroacetic acid in CH₂Cl₂ at room temperature gave an 86% yield of 2-(difluoromethyl)-1-[4-(4-morpholinyl)-6-(1-piperazinyl)-1,3,5-triazin-2-yl]-1H-benzoimidazol-4-ol: mp (MeOH) 269-271° C.; ¹H NMR (DMSO-d₆) δ 7.73 (d, J=8.2 Hz, 1H), 7.69 (t, J_{HF}=52.9 Hz, 1H), 7.25 (t, J=8.1 Hz, 1H), 6.76 (d, J=7.8 Hz, 1H), 3.74 (m, 12H), 2.76 (m, 4H); MS (APCI⁺) 433.9 (MH⁺); Anal. Calcd. for C₁₉H₂₂F₂N₈O₂: C, 52.8; H, 5.1; N, 25.9. Found: C, 52.7; H, 5.2; N, 25.85%.

A suspension of 0.22 g (0.5 mmol) of 2-(difluoromethyl)-1-[4-(4-morpholinyl)-6-(1-piperazinyl)-1,3,5-triazin-2-yl]-1H-benzoimidazol-4-ol in 200 mL CH₂Cl₂ containing 0.25 g (2.5 mmol) triethyamine was cooled to 0° C. where 0.23 g (2 mmol) of chloroacetyl chloride was added dropwise. The mixture was allowed to warm to room temperature for 2 hrs before being quenched with water. After being washed successively with dilute aqueous acetic acid and NaHCO₃ solutions, the CH₂Cl₂ was removed under vacuum and the residue was dissolved in a mixture of acetone and 2 M aqueous NaHCO₃. After 4 hrs the acetone was removed under vacuum and the residue was extracted into CH₂Cl₂. Chromatography on silica gel, eluting with CH₂Cl₂/EtOAc (3:2), gave 145 mg (53% yield) of 1-[4-[4-(chloroacetyl)-1-piperazinyl]-6-(4-morpholinyl)-1,3,5-triazin-2-yl]-2-(difluoromethyl)-1H-benzimidazol-4-ol: mp (CH₂Cl₂/MeOH) 267-270° C.; ¹H NMR (DMSO-d₆) δ 10.21 (br s, exchangeable with D₂O, 1H), 7.75 (d, J=8.2 Hz, 1H), 7.71 (t, J_{HF}=52.9 Hz, 1H), 7.27 (t, J=8.1 Hz, 1H), 6.77 (d, J=7.9 Hz, 1H), 4.45 (s, 2H), 3.86 (m, 2H), 3.82 (m, 6H), 3.69 (m, 4H), 3.61 (m, 4H); MS (APCI⁺) 510.1 (MH⁺); Anal. Calcd. for C₂₁H₂₃ClF₂N₈O₃: C, 49.6; H, 4.6; N, 22.0. Found: C, 49.35; H, 4.75; N, 22.0%.

### Example 6

### Synthesis of 1-[4-[4-(chloroacetyl)-1-piperazinyl]-6-(4-pyridinyl)-1,3,5-triazin-2-yl]-2-(difluoromethyl)-4-methoxy-1H-benzimidazole

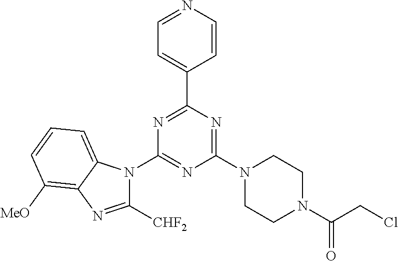

The compound was prepared according to Scheme 1 using Method A.

A mixture of 0.99 g (50 mmol) of 2-(difluoromethyl)-4-methoxy-1H-benzimidazole, 2.0 g tert-butyl 4-(4,6-dichloro-1,3,5-triazin-2-yl)piperazine-1-carboxylate (Lowik et al., Eur. J. Org. Chem., 2001, 2825), and 3.5 g (250 mmol) powdered K₂CO₃ in 40 mL DMF was stirred at room temperature for 1 hr. Water was added and the product was collected by filtration and washed with water and cold ethanol to give 2.14 g (86% yield) of tert-butyl 4-(4-chloro-6-(2-(difluoromethyl)-4-methoxy-1H-benzimidazol-1-yl)-1,3,5-triazin-2-yl)piperazine-1-carboxylate: mp (CH₂Cl₂/EtOH)>300° C.; ¹H NMR (CDCl₃) δ 7.99 (d, J=8.3 Hz, 1H), 7.48 (t, J_{HF}=53.4 Hz, 1H), 7.41 (t, J=8.3 Hz, 1H), 6.87 (d, J=8.0 Hz, 1H), 4.06 (s, 3H), 3.95 (m, 4H), 3.58 (m, 4H), 1.50 (s, 9H); MS (APCI⁺) 497.1/499.1 (MH⁺); Anal. Calcd. for C₂₁H₂₄ClF₂N₇O₃: C, 50.9; H, 4.9; N, 19.8. Found: C, 51.1; H, 4.9; N, 19.95%.

A mixture of 0.496 g (1 mmol) of tert-butyl 4-(4-chloro-6-(2-(difluoromethyl)-4-methoxy-1H-benzoimidazol-1-yl)-1,3,5-triazin-2-yl)piperazine-1-carboxylate, 0.18 g (1.5 mmol) 4-pyridyl boronic acid, 8 mL of 4 M Na₂CO₃ solution and 56 mg PdCl₂(dppf) in 40 mL dioxane was heated under reflux under an atmosphere of nitrogen for 1 hr. The dioxane was removed under vacuum and the product was collected and washed with water. Chromatography on alumina, eluting with CH₂Cl₂/EtOAc (95:5), gave 0.152 g (28% yield) of tert-butyl 4-(4-(2-(difluoromethyl)-4-methoxy-1H-benzimidazol-1-yl)-6-(4-pyridinyl)-1,3,5-triazin-2-yl)piperazine-1-carboxylate: mp (CH₂Cl₂/MeOH) 242-245° C.; ¹H NMR (CDCl₃) δ 8.86 (dd, J=4.5, 1.5 Hz, 2H), 8.27 (dd, J=4.5, 1.6 Hz, 2H), 8.07 (d, J=8.3 Hz, 1H), 7.52 (t, J_{HF}=53.4 Hz, 1H), 7.45 (t, J=8.2 Hz, 1H), 6.88 (d, J=8.0 Hz, 1H), 4.13 (m, 2H), 4.08 (s, 3H) 4.00 (m, 2H), 3.63 (m, 4H), 1.52 (s, 9H); MS (APCI⁺) 540.4 (MH⁺); Anal. Calcd. for C₂₆H₂₈F₂N₈O₃: C, 58.0; H, 5.2; N, 20.8. Found: C, 57.9; H, 5.2; N, 21.0%.

A solution of 0.12 g (0.22 mmol) tert-butyl 4-(4-(2-(difluoromethyl)-4-methoxy-1H-benzimidazol-1-yl)-6-(4-pyridinyl)-1,3,5-triazin-2-yl)piperazine-1-carboxylate was treated with an excess of trifluoroacetic acid in CH₂Cl₂ to give crude 2-(difluoromethyl)-4-methoxy-1-[4-(piperazin-1-yl)-6-(4-pyridinyl)-1,3,5-triazin-2-yl]-1H-benzimidazole: ¹H NMR (DMSO-d₆) δ 8.87 (dd, J=4.4, 1.6 Hz, 2H), 8.30 (dd, J=4.5, 1.6 Hz, 2H), 8.06 (d, J=7.9 Hz, 1H), 7.81 (t, J_{HF}=52.7 Hz, 1H), 7.49 (t, J=8.2 Hz, 1H), 7.01 (d, J=7.9 Hz, 1H), 6.20 (br m, exchangeable with D₂O, 1H), 4.10 (m, 2H), 4.00 (s, 3H), 3.95 (m, 2H), 3.01 (m, 4H); MS (APCI⁺) 440.0 (MH⁺).

A solution of crude 2-(difluoromethyl)-4-methoxy-1-[4-(piperazin-1-yl)-6-(4-Pyridinyl)-1,3,5-triazin-2-yl]-1H-benzimidazole and diisopropylethylamine in CH₂Cl₂ was treated with chloroacetyl chloride at 0° C. After 30 min the solution was washed successively with dilute aqueous acetic acid and NaHCO₃ solution, and dried. The solution was eluted through a short column of alumina and HCl in methanol was added. The solvent was removed and the residue was recrystallized from methanol to give 1-[4-[4-(chloroacetyl)-1-piperazinyl]-6-(4-pyridinyl)-1,3,5-triazin-2-yl]-2-(difluoromethyl)-4-methoxy-1H-benzimidazole hydrochloride: mp>300° C.; ¹H NMR (DMSO-d₆) δ 8.91 (d, J=6.0 Hz, 2H), 8.37 (d, J=6.1 Hz, 2H), 8.09 (d, J=8.3 Hz, 1H), 7.84 (t, J_{HF}=52.6 Hz, 1H), 7.51 (t, J=8.2 Hz, 1H), 7.03 (d, J=8.1 Hz, 1H), 4.50 (s, 2H), 4.16 (m, 2H), 4.00 (s, 3H), 3.98 (m, 2H), 3.71 (m, 4H); MS (APCI⁺) 516.2/518.2 (MH⁺); Anal. Calcd. for C₂₃H₂₂O₂F₂N₈O₂: C, 50.1; H, 4.0; N, 20.3. Found: C, 49.9; H, 4.1; N, 20.2%.

### Example 7

### Synthesis of 1-[4-[4-(dichloroacetyl)-1-piperazinyl]-6-(4-morpholinyl)-1,3,5-triazin-2-yl]-2-(difluoromethyl)-4-methoxy-1H-benzimidazole

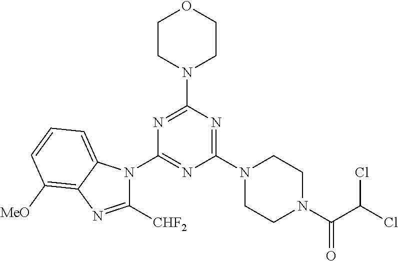

A mixture of 1.98 g (5 mmol) of 1-[4-chloro-6-(4-morpholinyl)-1,3,5-triazin-2-yl]-2-(difluoromethyl)-4-methoxy-1H-benzimidazole, 1.16 g (6.25 mmol) of tert-butyl 1-piperazinecarboxylate, and 1.29 g (10 mmol) of DIPEA in 100 mL of THF was stirred at room temperature for 1 hr, and the solution was concentrated under vacuum. The residue was diluted with water containing 1 mL of acetic acid and the resulting precipitate was collected, washed with water, and dried, to give 2.71 g, (99% yield) of tert-butyl 4-[4-[2-(difluoromethyl)-4-methoxy-1H-benzimidazol-1-yl]-6-(4-morpholinyl)-1,3,5-triazin-2-yl]-1-piperazinecarboxylate: mp (MeOH) 221-223° C.; ¹H NMR (CDCl₃) δ 7.88 (dd, J=8.4, 0.6 Hz, 1H), 7.47 (t, J_{HF}=53.5 Hz, 1H), 7.35 (t, J=8.2 Hz, 1H), 6.81 (d, J=7.7 Hz, 1H), 4.05 (s, 3H), 3.87 (m, 8H), 3.78 (m, 4H), 3.53 (m, 4H), 1.50 (s, 9H); Anal. Calcd. for C₂₅H₃₂F₂N₈O₄: C, 54.9; H, 5.9; N, 20.5. Found: C, 54.9; H, 5.9; N, 20.5%.

Reaction of the above carbamate with an excess of TFA (10 mL) in CH₂Cl₂ at room temperature for 2 hrs, followed by treatment with aq. NH₃ gave 2-(difluoromethyl)-4-methoxy-1-[4-(4-morpholinyl)-6-(1-piperazinyl)-1,3,5-triazin-2-yl]-1H-benzimidazole in 100% yield: mp (EtOH) 228-231° C.: ¹H NMR (CDCl₃) δ 7.90 (d, J=7.9 Hz, 1H), 7.50 (t, J_{HF}=53.5 Hz, 1H), 7.34 (t, J=8.3 Hz, 1H), 6.81 (d, J=7.8 Hz, 1H), 4.05 (s, 3H), 3.87 (m, 8H), 3.78 (m, 4H), 2.95 (m, 4H); Anal. Calcd. for C₂₀H₂₄F₂N₈O₂: C, 53.8; H, 5.4; N, 25.1. Found: C, 53.8; H, 5.6; N, 25.3%.

Reaction of the above amine with dichloroacetyl chloride in CH₂Cl₂ gave 1-[4-[4-(dichloroacetyl)-1-piperazinyl]-6-(4-morpholinyl)-1,3,5-triazin-2-yl]-2-(difluoromethyl)-4-methoxy-1H-benzimidazole in 97% yield: mp (CH₂Cl₂/MeOH) 275-278° C.; ¹H NMR (DMSO-d₆) δ 7.90 (d, J=8.0 Hz, 1H), 7.70 (t, J_{HF}=52.8 Hz, 1H), 7.41 (t, J=8.2 Hz, 1H), 7.29 (s, 1H), 6.96 (d, J=7.8 Hz, 1H), 3.98 (s, 3H), 3.89-3.88 (m, 8H), 3.70 (m, 8H); Anal. Calcd. for C₂₂H₂₄Cl₂F₂N₈O₃.0.15CH₂Cl₂: C, 46.7; H, 4.3; N, 19.7. Found: C, 46.8, H, 4.3; N, 19.6%.

### Example 8

### Synthesis of 2-(difluoromethyl)-1-{4-(4-morpholinyl)-6-[4-(trichloroacetyl)-1-piperazinyl]-1,3,5-triazin-2-yl}-1H-benzimidazol-4-yl methyl ether

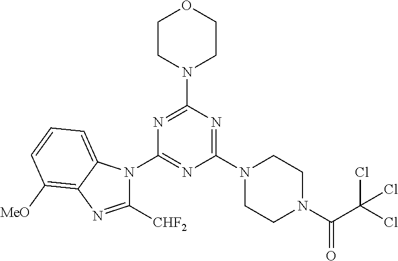

Reaction of 2-(difluoromethyl)-4-methoxy-1-[4-(4-morpholinyl)-6-(1-piperazinyl)-1,3,5-triazin-2-yl]-1H-benzimidazole with trichloroacetyl chloride in CH₂Cl₂ gave 2-(difluoromethyl)-1-{4-(4-morpholinyl)-6-[4-(trichloroacetyl)-1-piperazinyl]-1,3,5-triazin-2-yl}-1H-benzimidazol-4-yl methyl ether in 87% yield: mp (CH₂Cl₂/MeOH) 252° C. (dec); ¹H NMR (DMSO-d₆) δ 7.90 (d, J=8.0 Hz, 1H), 7.71 (t, J_{HF}=52.8 Hz, 1H), 7.42 (t, J=8.2 Hz, 1H), 6.96 (d, J=7.8 Hz, 1H), 3.98 (s, 3H), 3.96-3.70 (m, 16H); Anal. Calcd. for C₂₂H₂₃Cl₃F₂N₈: C, 44.7; H, 3.9; N, 18.9. Found: C, 44.9; H, 3.9; N, 19.0%.

### Example 9

### Synthesis of 2-{4-[4-[2-(difluoromethyl)-4-methoxy-1H-benzimidazol-1-yl]-6-(4-morpholinyl)-1,3,5-triazin-2-yl]-1-piperazinyl}-2-oxoethyl methanesulfonate

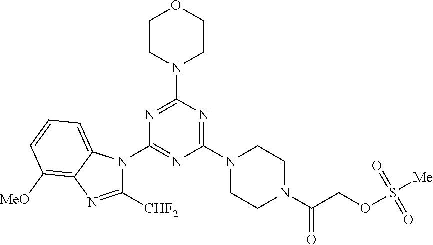

A mixture of 2-(difluoromethyl)-4-methoxy-1-[4-(4-morpholinyl)-6-(1-piperazinyl)-1,3,5-triazin-2-yl]-1H-benzimidazole (403 mg, 0.9 mmol) and DIPEA (1 mL) in CH₂Cl₂ (25 mL) was treated with acetoxyacetyl chloride (1 mL) at 0° C. The reaction mixture was allowed to warm to room temperature, and after being stirred for 4 hrs it was diluted with water (30 mL). The organic layer was separated, dried (Na₂SO₄), and evaporated to give a crude product which was chromatographed on silica, eluting with CH₂Cl₂/EtOAc (4:1) to give 2-{4-[4-[2-(difluoromethyl)-4-methoxy-1H-benzimidazol-1-yl]-6-(4-morpholinyl)-1,3,5-triazin-2-yl]-1-piperazinyl}-2-oxoethyl acetate in 85% yield: ¹H NMR (DMSO-d₆) δ 7.90 (d, J=7.9 Hz, 1H), 7.70 (t, J_{HF}=52.8 Hz, 1H), 7.41 (t, J=8.2 Hz), 6.96 (d, J=7.8 Hz, 1H), 4.84 (s, 2H), 3.97 (s, 3H), 3.87-3.89 (m, 8H), 3.87-3.81 (m, 8H), 3.71-3.70 (m, 4H), 3.56 (m, 4H), 2.03 (s, 3H).

The above acetate (387 mg, 0.71 mmol) was dissolved in a mixture of THF (10 mL) and H₂O (4 mL), and Cs₂CO₃ (829 mg, 7.1 mmol) was added. The reaction mixture was refluxed for 48 hrs, cooled to 20° C. and diluted with water. The resulting precipitate was filtered, washed with water, and chromatographed on silica eluting first with CH₂Cl₂/EtOAc (7:3) then with CH₂Cl₂/MeOH (49:1) to give 2-{4-[4-[2-(difluoromethyl)-4-methoxy-1H-benzimidazol-1-yl]-6-(4-morpholinyl)-1,3,5-triazin-2-yl]-1-piperazinyl}-2-oxoethanol in 85% yield: ¹H NMR (DMSO-d₆) δ 7.90 (d, J=8.36 Hz, 1H) 7.70 (t, J_{HF}=52.8 Hz, 1H), 7.41 (t, J=8.2 Hz, 1H), 6.96 (d, J=7.8 Hz, 1H), 4.63 (t, J=5.2 Hz, exchangeable with D₂O, 1H), 4.15 (d, J=5.2 Hz, 2H), 3.97 (s, 3H), 3.84-3.81 (m, 8H), 3.71-3.70 (m, 4H), 3.60-3.49 (m, 4H).

The above alcohol (158 mg, 0.31 mmol) and Et₃N (0.16 mL) were dissolved in dry CH₂Cl₂ (10 mL), and cooled in an ice bath. Methanesulphonyl chloride (0.05 mL) was added, and the mixture was allowed to warm to room temperature with stirring over 2 hrs. The reaction mixture was diluted with water (10 mL) and the organic layer was separated, dried (Na₂SO₄), and evaporated to give a 92% yield of 2-{4-[4-[2-(difluoromethyl)-4-methoxy-1H-benzimidazol-1-yl]-6-(4-morpholinyl)-1,3,5-triazin-2-yl]-1-piperazinyl}-2-oxoethyl methanesulfonate as a white solid: mp (CH₂Cl₂/hexanes) 176-180° C.; ¹H NMR (DMSO-d₆) δ 7.90 (d, J=8.4 Hz, 1H), 7.70 (t, J_{HF}=52.8 Hz, 1H), 7.41 (t, J=8.1 Hz, 1H), 6.96 (d, J=7.8 Hz, 1H), 5.09 (s, 2H), 3.97 (s, 3H), 3.88-3.82 (m, 8H), 3.70 (m, 4H), 3.59-3.40 (m, 2H), 3.28 (s, 3H); Anal. Calcd. for C₂₃H₂₈F₂N₈O₆S.H₂O: C, 46.0; H, 5.0; N, 18.7. Found: C, 46.2; H, 5.1; N, 17.9%.

### Example 10

### Synthesis of 1-[4-[4-(2-chloropropanoyl)-1-piperazinyl]-6-(4-morpholinyl)-1,3,5-triazin-2-yl]-2-(difluoromethyl)-4-methoxy-1H-benzimidazole

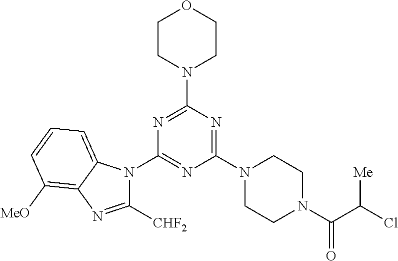

Reaction of 2-(difluoromethyl)-4-methoxy-1-[4-(4-morpholinyl)-6-(1-piperazinyl)-1,3,5-triazin-2-yl]-1H-benzimidazole with 2-chloropropionyl chloride in CH₂Cl₂ as above gave 1-[4-[4-(2-chloropropanoyl)-1-piperazinyl]-6-(4-morpholinyl)-1,3,5-triazin-2-yl]-2-(difluoromethyl)-4-methoxy-1H-benzimidazole in 89% yield: ¹H NMR (DMSO-d₆) [symbol] 7.90 (d, J=8.1 Hz, 1H), 7.70 (t, J_{HF}=52.8 Hz, 1H), 7.41 (t, J=8.2 Hz, 1H), 6.96 (d, J=7.8 Hz, 1H), 5.12 (d, J=5.9 Hz, 1H), 3.98 (s, 3H), 3.88-3.82 (m, 8H), 3.71-3.63 (m, 8H), 1.55 (d, J=6.4 Hz, 3H); Anal. Calcd. for C₂₃H₂₇ClF₂N₈O₃: C, 51.45; H, 5.1; N, 20.9. Found: C, 51.6; H, 5.2; N, 21.0%.

### Example 11

### Synthesis of 1-[4-{4-[(chloromethyl)sulfonyl]-1-piperazinyl}-6-(4-morpholinyl)-1,3,5-triazin-2-yl]-2-(difluoromethyl)-4-methoxy-1H-benzimidazole

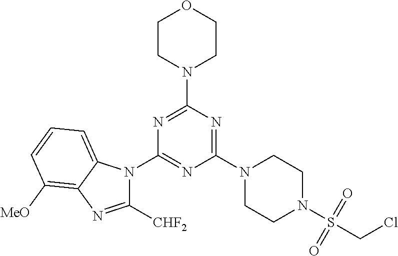

The compound was prepared according to Scheme 1 using Method A.

A solution of 224 mg (0.5 mmol) of 2-(difluoromethyl)-4-methoxy-1-[4-(4-morpholinyl)-6-(1-piperazinyl)-1,3,5-triazin-2-yl]-1H-benzimidazole in 10 mL of pyridine was cooled to 0° C. and 0.112 g (0.75 mmol) of chloromethanesulfonyl chloride was added over 5 min. The mixture was allowed to warm to room temperature. After 1 hr, it was diluted with water to give a precipitate which was collected and dried. Chromatography on silica eluting with CH₂Cl₂/EtOAc (4:1) gave 100 mg (36% yield) of 1-[4-{4-[(chloromethyl)sulfonyl]-1-piperazinyl}-6-(4-morpholinyl)-1,3,5-triazin-2-yl]-2-(difluoromethyl)-4-methoxy-1H-benzimidazole: mp (MeOH) 261-263° C.; ¹H NMR (CDCl₃) δ 7.85 (d, J=8.3 Hz, 1H), 7.43 (t, J_{HF}=53.5 Hz, 1H), 7.35 (t, J=8.2 Hz, 1H), 6.82 (d, J=8.0 Hz, 1H), 4.55 (s, 2H), 4.05 (s, 3H), 4.00 (m, 4H), 3.88 (m, 4H), 3.79 (m, 4H), 3.57 (m, 4H); Anal. Calcd. for C₂₁H₂₅ClF₂N₈O₄S: C, 45.1; H, 4.5; N, 20.05. Found: C, 45.3; H, 4.5; N, 20.2%.

### Example 12

### Synthesis of 1-[4-{4-[(bromomethyl)sulfonyl]-1-piperazinyl}-6-(4-morpholinyl)-1,3,5-triazin-2-yl]-2-(difluoromethyl)-4-methoxy-1H-benzimidazole

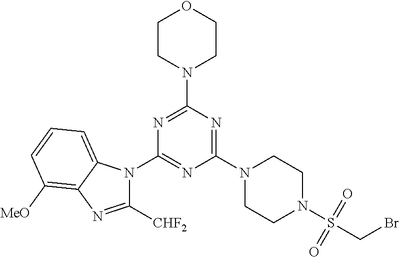

The compound was prepared according to Scheme 1 using Method A.

A solution of 0.5 g (1.1 mmol) of 2-(difluoromethyl)-4-methoxy-1-[4-(4-morpholinyl)-6-(1-piperazinyl)-1,3,5-triazin-2-yl]-1H-benzimidazole and a suspension of 0.8 g powdered K₂CO₃ in CH₂Cl₂ (50 mL) was cooled to 0° C. and 0.8 g (3.3 mmol) of bromomethylsulfonyl bromide (Block and Aslam, Org. Synth. Coll. Vol. 1987, 8, 212) was added. The stirred mixture was allowed to warm to room temperature overnight, and was diluted with water and aq. NH₃. After drying (Na₂SO₄) and removal of the solvent, the white solid was chromatographed on silica, eluting with CH₂Cl₂/EtOAc (9:1) to give 335 mg (50% yield) of 1-[4-{4-[(bromomethyl)sulfonyl]-1-piperazinyl}-6-(4-morpholinyl)-1,3,5-triazin-2-yl]-2-(difluoromethyl)-4-methoxy-1H-benzimidazole: mp (MeOH) 259-262° C.; ¹H NMR (CDCl₃) δ 7.86 (dd, J=8.4, 0.6 Hz, 1H), 7.44 (t, J_{HF}=53.5 Hz, 1H), 7.36 (t, J=8.2 Hz, 1H), 6.83 (d, J=7.8 Hz, 1H), 4.46 (s, 2H), 4.05 (s, 3H), 4.01 (m, 4H), 3.89 (m, 4H), 3.79 (m, 4H), 3.58 (m, 4H); Anal. Calcd. for C₂₁H₂₅BrF₂N₈O₄S: C, 41.8; H, 4.2; N, 18.6. Found: C, 41.8; H, 4.4; N, 18.6%.

### Example 13

### Synthesis of N-(3-{[1-[4-{4-[(chloromethyl)sulfonyl]-1-piperazinyl}-6-(4-morpholinyl)-1,2,5-triazin-2-yl]-2-(difluoromethyl)-1H-benzimidazol-4-yl]oxy}propyl)-N,N-dimethylamine

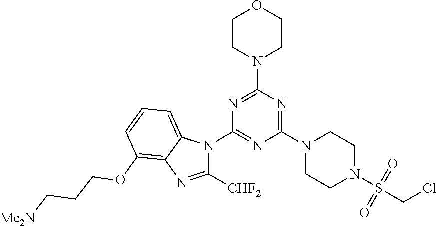

The compound was prepared according to Scheme 1 using Method A.

A mixture of 0.60 g (1.1 mmol) of tert-butyl 4-[4-[2-(difluoromethyl)-4-hydroxy-1H-benzimidazol-1-yl]-6-(4-morpholinyl)-1,3,5-triazin-2-yl]piperazine-1-carboxylate (Example 5), 0.47 g (3.3 mmol) of 3-bromo-1-propanol, and 0.80 g (5.5 mmol) of powdered K₂CO₃ in 20 mL dry DMF was stirred at room temperature for 8 hrs. Dilution with water gave 0.66 g (99% yield) of tert-butyl 4-[4-[2-(difluoromethyl)-4-(3-hydroxypropoxy)-1H-benzimidazol-1-yl]-6-(4-morpholinyl)-1,3,5-triazin-2-yl]-1-piperazinecarboxylate: ¹H NMR (CDCl₃) δ 7.94 (dd, J=8.4. 0.7 Hz, 1H), 7.49 (t, J_{HF}=53.4 Hz, 1H), 7.34 (t, J=8.1 Hz, 1H), 6.92 (dd, J=8.0, 0.6 Hz, 1H), 4.47 (t, J=5.9 Hz, 2H), 3.98 (t, J=5.4 Hz, 2H), 3.87 (m, 8H), 3.79 (m, 4H), 3.54 (m, 4H), 3.30 (m, exchangeable with D₂O, 1H), 2.14 (pentet, J=5.8 Hz, 2H), 1.50 (s, 9H).

A mixture of the above alcohol and 0.34 g (3.3 mmol) of Et₃N in 20 mL of THF was cooled to 0° C. and 0.32 g (2.8 mmol) of methanesulfonyl chloride was added dropwise. After 1 hr, 6 g of 40% aqueous Me₂NH was added, and the resulting mixture was stirred at room temperature for 36 hr. The THF was removed under vacuum and the residue was diluted with water and extracted into CH₂Cl₂. Drying and removal of the solvent gave tert-butyl 4-[4-{2-(difluoromethyl)-4-[3-(dimethylamino)propoxy]-1H-benzimidazol-1-yl}-6-(4-morpholinyl)-1,3,5-triazin-2-yl]-1-piperazinecarboxylate as an oil: ¹H NMR (CDCl₃) δ 7.87 (dd, J=8.4. 0.6 Hz, 1H), 7.48 (t, J_{HF}=53.5 Hz, 1H), 7.33 (t, J=8.2 Hz, 1H), 6.85 (d, J=7.8 Hz, 1H), 4.31 (t, J=6.7 Hz, 2H), 3.87 (m, 8H), 3.79 (m, 4H), 3.53 (m, 4H), 2.51 (t, J=7.2 Hz, 2H), 2.26 (s, 6H), 2.13 (pentet, J=7.0 Hz, 2H), 1.50 (s, 9H).

Treatment of the above crude carbamate with TFA in CH₂Cl₂ gave N-[3-({2-(difluoromethyl)-1-[4-(4-morpholinyl)-6-(1-piperazinyl)-1,3,5-triazin-2-yl]-1H-benzimidazol-4-yl}oxy)propyl]-N,N-dimethylamine as a solid: ¹H NMR (CDCl₃) δ 7.89 (dd, J=8.4. 0.7 Hz, 1H), 7.50 (t, J_{HF}=53.5 Hz, 1H), 7.31 (t, J=8.2 Hz, 1H), 6.84 (d, J=8.0 Hz, 1H), 4.32 (t, J=6.8 Hz, 2H), 3.86 (m, 8H), 3.78 (m, 4H), 2.95 (m, 4H), 2.53 (t, J=7.2 Hz, 2H), 2.27 (s, 6H), 2.13 (pentet, J=6.9 Hz, 2H).

A mixture of 0.42 g (0.81 mmol) of the above amine and 1 g powdered K₂CO₃ in CH₂Cl₂ was cooled to 0° C. and 0.36 g (2.4 mmol) of chloromethanesulfonyl chloride was added dropwise. The mixture was allowed to warm to room temperature, and after 1 hr water was added. The solvent was dried, and the solution was absorbed on to a column of alumina. Non-polar impurities were removed by elution with CH₂Cl₂/EtOAc 9:1 and fractions containing the product were obtained by subsequent elution with CH₂Cl₂/EtOAc 1:1. The solution was acidified with HCl in MeOH (1.25 M), and the solvents were removed under vacuum. The resulting solid was recrystallised from EtOH/EtOAc to give N-(3-{[1-[4-{4-[(chloromethyl)sulfonyl]-1-piperazinyl}-6-(4-morpholinyl)-1,3,5-triazin-2-yl]-2-(difluoromethyl)-1H-benzimidazol-4-yl]oxy}propyl)-N,N-dimethylamine hydrochloride: mp (EtOH/EtOAc) 243-247° C.; ¹H NMR (DMSO-d₆) δ 10.26 (m, exchangeable with D₂O, 1H), 7.92 (d, J=8.3 Hz, 1H), 7.70 (t, J_{HF}=52.8 Hz, 1H), 7.42 (t, J=8.2 Hz, 1H), 6.99 (d, J=7.9 Hz, 1H), 4.33 (t, J=6.1 Hz, 2H), 3.94 (m, 4H), 3.82 (m, 4H), 3.70 (m, 4H), 3.29 (m, 2H), 3.22 (m, 4H), 2.91 (s, 3H), 2.81 (s, 6H), 2.25 (m, 2H); Anal. Calcd. for C₂₅H₃₅Cl₂F₂N₉O₄S.H₂O: C, 43.9; H, 5.45; Cl, 10.4; N, 18.4. Found: C, 43.5; H, 5.5; Cl, 10.0; N, 18.3%.

### Example 14

### Synthesis of 1-[4-{4-[(chloromethyl)sulfonyl]-1-piperazinyl}-6-(4-morpholinyl)-2-pyrimidinyl]-2-(difluoromethyl)-4-methoxy-1H-benzimidazole

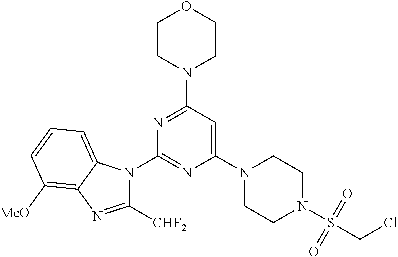

The compound was prepared according to Scheme 1 using Method A.

A solution of 0.223 g (0.5 mmol) of o 2-(difluoromethyl)-4-methoxy-1-[4-(4-morpholinyl)-6-(1-piperazinyl)-2-pyrimidinyl]-1H-benzimidazole (WO 2008/032064) and 0.10 g (1 mmol) of Et₃N in 20 mL CH₂Cl₂ was cooled to 0° C., and 0.112 g (0.75 mmol) of chloromethanesulfonyl chloride was added dropwise. The reaction mixture was allowed to warm to room temperature and, after 1 hr, water was added. The organic layer was separated, washed successively with aqueous acetic acid and aq. ammonia, and dried. Polar impurities were removed by elution through a column of alumina with CH₂Cl₂, and the solvent was then removed to give 0.197 g (71% yield) of 1-[4-{4-[(chloromethyl)sulfonyl]-1-piperazinyl}-6-(4-morpholinyl)-2-pyrimidinyl]-2-(difluoromethyl)-4-methoxy-1H-benzimidazole: mp (MeOH) 229-232° C.; ¹H NMR (CDCl₃) δ 7.74 (d, J=7.9 Hz, 1H), 7.37 (t, J_{HF}=53.5 Hz, 1H), 7.32 (t, J=8.2 Hz, 1H), 6.79 (t, J=7.9 Hz, 1H), 5.53 (s, 1H), 4.55 (s, 2H), 4.05 (s, 3H), 3.82 (m, 4H), 3.78 (m, 4H), 3.64 (m, 4H), 3.59 (m, 4H); Anal. Calcd. for C₂₂H₂₆ClF₂N₇O₄S: C, 47.35; H, 4.7; N, 17.6. Found: C, 47.5; H, 4.8; N, 17.5%.

### Example 15

### Synthesis of 1-[6-{4-[(chloromethyl)sulfonyl]-1-piperazinyl}-2-(4-morpholinyl)-4-pyrimidinyl]-2-(difluoromethyl)-4-methoxy-1H-benzimidazole

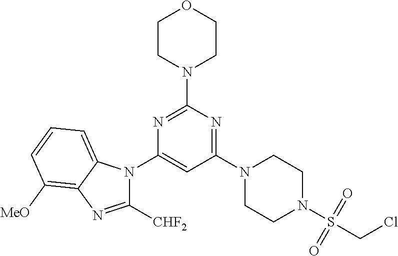

The compound was prepared according to Scheme 1 using Method A.

A mixture of 7.72 g (0.04 mol) 4,6-dichloro-2-(methylsulfanyl)pyrimidine, 7.93 g (0.04 mol) 2-(difluoromethyl)-4-methoxy-1H-benzimidazole, and 22 g (0.26 mol) of powdered K₂CO₃ in 100 mL DMSO was stirred at room temperature for 3 days and diluted with water. The solid was collected, washed with water, and dried. Chromatography on silica, eluting with CH₂Cl₂/EtOAc (95:5) gave 5.91 g (41% yield) of 1-[6-chloro-2-(methylsulfanyl)-4-pyrimidinyl]-2-(difluoromethyl)-4-methoxy-1H-benzimidazole: mp (i-Pr₂O/hexanes) 120-121° C.; ¹H NMR (CDCl₃) δ 7.40 (t, J=8.2 Hz, 1H), 7.32 (s, 1H), 7.26 (dd, J=8.4, 0.7 Hz, 1H), 7.18 (t, J_{HF}=53.3 Hz, 1H), 6.85 (d, J=7.9 Hz, 1H), 4.07 (s, 3H), 2.62 (s, 3H); MS (APCI+) m/z 357.6 (MH⁺); Anal. Calcd. for C₁₄H₁₁ClF₂N₄OS: C, 47.1; H, 3.1; N, 15.7. Found C, 47.3; H, 3.4; N, 15.7%. Further elution with CH₂Cl₂/EtOAc (9:1) gave 4.16 g (20% yield) of the bis-addition byproduct, 2-(difluoromethyl)-1-[6-[2-(difluoromethyl)-4-methoxy-1H-benzimidazol-1-yl]-2-(methylsulfanyl)-4-pyrimidinyl]-4-methoxy-1H-benzimidazole: ¹H NMR (CDCl₃) δ 7.64 (s, 1H), 7.45-7.38 (m, 4H), 7.26 (t, J_{HF}=53.3 Hz, 2H), 6.87 (dd, J=6.9, 2.0 Hz, 2H), 4.07 (s, 6H), 2.62 (s, 3H).

A mixture of 2 g (5.6 mmol) of 1-[6-chloro-2-(methylsulfanyl)-4-pyrimidinyl]-2-(difluoromethyl)-4-methoxy-1H-benzimidazole and 2.6 g (14 mmol) of tert-butyl 1-piperazinecarboxylate in 50 mL of THF was stirred at room temperature for 2 hrs before being diluted with water containing 1 mL of acetic acid. The precipitate was collected, dried (Na₂SO₄), and chromatographed on silica, eluting with CH₂Cl₂/EtOAc (95:5) to give 2.72 g (96% yield) of tert-butyl 4-[6-[2-(difluoromethyl)-4-methoxy-1H-benzimidazol-1-yl]-2-(methylsulfanyl)-4-pyrimidinyl]-1-piperazinecarboxylate: mp (MeOH) 160-161° C.; ¹H NMR (CDCl₃) δ 7.32 (t, J=8.1 Hz, 1H), 7.22 (dd, J=8.4, 0.7 Hz, 1H), 7.18 (t, J_{HF}=53.4 Hz, 1H), 6.79 (dd, J=7.9, 0.5 Hz, 1H), 6.43 (s, 1H), 4.06 (s, 3H), 3.72 (m, 4H), 3.57 (m, 4H), 2.54 (s, 3H), 1.49 (s, 9H); Anal. Calcd. for C₂₃H₂₈F₂N₆O₃S: C, 54.5; H, 5.6; N, 16.6. Found: C, 54.6; H, 5.5; N, 16.6%.

A solution of 2.53 g (50 mmol) of the above compound in a mixture of 500 mL of acetone and 50 mL of acetic acid was treated with a solution of 5 g KMnO₄ in water (100 mL) in portions over 5 min. The resulting mixture was stirred at room temperature for 3 hrs, decolourized with aqueous Na₂SO₃, and diluted with water, to give a white solid which was extracted with CH₂Cl₂, washed with water, and dried, to give 1.80 g (67% yield) of tert-butyl 4-[6-[2-(difluoromethyl)-4-methoxy-1H-benzimidazol-1-yl]-2-(methylsulfonyl)-4-pyrimidinyl]-1-piperazinecarboxylate: mp (MeOH) 199° C. (dec.); ¹H NMR (CDCl₃) δ 7.38 (t, J=8.2 Hz, 1H), 7.26 (d, J=8.3 Hz, 1H), 7.13 (t, J_{HF}=53.2 Hz, 1H), 6.87 (s, 1H), 6.84 (d, J=7.6 Hz, 1H), 4.07 (s, 3H), 3.80 (m, 4H), 3.62 (m, 4H), 3.31 (s, 3H), 1.50 (s, 9H); Anal. Calcd. for C₂₃H₂₈F₂N₆O₅S: C, 51.3; H, 5.2; N, 15.6. Found: C, 51.4; H, 5.3; N, 15.5%.

A solution of 1.077 g (2 mmol) of the above sulfone and 0.87 g (10 mmol) of morpholine in 50 mL of THF was heated under reflux overnight, and, after cooling, water was added, to give 1.09 g (100% yield) of tert-butyl 4-[6-[2-(difluoromethyl)-4-methoxy-1H-benzimidazol-1-yl]-2-(4-morpholinyl)-4-pyrimidinyl]-1-piperazinecarboxylate: mp (MeOH) 177-179° C.; ¹H NMR (CDCl₃) δ 7.31 (t, J=8.1 Hz, 1H), 7.22 (dd, J=8.3, 0.7 Hz, 1H), 7.15 (t, J_{HF}=53.4 Hz, 1H), 6.78 (d, J=7.4 Hz, 1H), 6.09 (s, 1H), 4.05 (s, 3H), 3.81-3.74 (m, 8H), 3.67 (m, 4H), 3.56 (m, 4H), 1.49 (s, 9H); Anal. Calcd. for C₂₆H₃₃F₂N₇O₄: C, 57.2; H, 6.1; N, 18.0. Found: 57.3; H, 6.2; N, 18.0%.

Treatment of the above carbamate with TFA in CH₂Cl₂ gave 2-(difluoromethyl)-4-methoxy-1-[2-(4-morpholinyl)-6-(1-piperazinyl)-4-pyrimidinyl]-1H-benzimidazole which was treated with chloromethanesulfonyl chloride as above. Chromatography on silica, eluting with CH₂Cl₂/EtOAc (9:1) gave 1-[6-{4-[(chloromethyl)sulfonyl]-1-piperazinyl}-2-(4-morpholinyl)-4-pyrimidinyl]-2-(difluoromethyl)-4-methoxy-1H-benzimidazole in 72% yield: mp (MeOH) 220-222° C.; ¹H NMR (CDCl₃) δ 7.31 (t, J=8.1 Hz, 1H), 7.21 (d, J=8.3 Hz, 1H), 7.14 (t, J_{HF}=53.3 Hz, 1H), 6.79 (d, J=7.9 Hz, 1H), 6.12 (s, 1H), 4.55 (s, 2H), 4.06 (s, 3H), 3.81-3.74 (m, 12H), 3.58 (m, 4H); Anal. Calcd. for C₂₂H₂₆ClF₂N₇O₄S: C, 47.35; H, 4.7; N, 17.6. Found: C, 47.5; H, 4.7; N, 17.8%.

### Example 16

### Synthesis of 1-[2-{4-[(chloromethyl)sulfonyl]-1-piperazinyl}-6-(4-morpholinyl)-4-pyrimidinyl]-2-(difluoromethyl)-4-methoxy-1H-benzimidazole

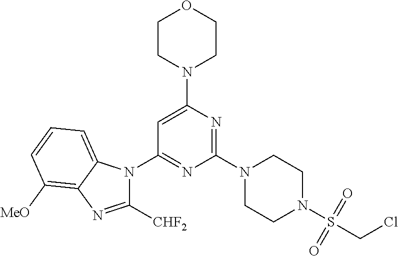

The compound was prepared according to Scheme 1 using Method A.

A mixture of 3.57 g (10 mmol) of 1-[6-chloro-2-(methylsulfanyl)-4-pyrimidinyl]-2-(difluoromethyl)-4-methoxy-1H-benzimidazole (see previous example) and 2.18 g (25 mmol) of morpholine in 50 mL THF was stirred at room temperature for 30 min, and diluted with water, to give 3.85 g (94% yield) of 2-(difluoromethyl)-4-methoxy-1-[2-(methylsulfanyl)-6-(4-morpholinyl)-4-pyrimidinyl]-1H-benzimidazole: mp (MeOH) 169-171° C.; ¹H NMR (CDCl₃) δ 7.32 (t, J=8.2 Hz, 1H), 7.22 (dd, J=8.4, 0.7 Hz, 1H), 7.18 (t, J_{HF}=53.4 Hz, 1H), 6.79 (d, J=7.5 Hz, 1H), 6.42 (s, 1H), 4.06 (s, 3H), 3.82 (m, 4H), 3.71 (m, 4H), 2.54 (s, 3H); Anal. Calcd. for C₁₈H₁₉F₂N₅O₂S: C, 53.1; H, 4.7; N, 17.2. Found: C, 53.1; H, 4.7; N, 17.3%.

A solution of 2.04 g (5 mmol) of the above sulfide in a mixture of 500 mL acetone and 50 mL acetic acid was combined with a solution of 5 g KMnO₄ in 100 mL water and the resulting mixture was stirred at room temperature for 1 hr. Dilution with water and decolourization with NaHSO₃ gave 1.80 g (82% yield) of 2-(difluoromethyl)-4-methoxy-1-[2-(methylsulfonyl)-6-(4-morpholinyl)-4-pyrimidinyl]-1H-benzimidazole as a white solid: mp (MeOH) 190-191° C.; ¹H NMR (CDCl₃) δ 7.38 (t, J=8.2 Hz, 1H), 7.26 (dd, J=8.4, 0.7 Hz, 1H), 7.13 (t, J_{HF}=53.2 Hz, 1H), 6.86 (s, 1H), 6.83 (d, J=7.8 Hz, 1H), 4.07 (s, 6H), 3.85 (m, 4H), 3.31 (s, 3H); Anal. Calcd. for C₁₈H₁₉F₂N₅O₄S: C, 49.2; H, 4.4; N, 15.9. Found: C, 49.4; H, 4.25; N, 15.9%.

A mixture of 1.099 g (2.5 mmol) of the above sulfone and 1.16 g (6.25 mmol) of tert-butyl 1-piperazinecarboxylate in 50 mL of THF was heated at reflux overnight before being diluted with water containing 1 mL of acetic acid. The product was extracted into CH₂Cl₂, washed with aq. NH₃, and dried. Chromatography on silica, eluting with CH₂Cl₂/EtOAc (9:1) gave 1.23 g (90% yield) of tert-butyl 4-[4-[2-(difluoromethyl)-4-methoxy-1H-benzimidazol-1-yl]-6-(4-morpholinyl)-2-pyrimidinyl]-1-piperazinecarboxylate: mp (hexanes) 149-152° C.; ¹H NMR (CDCl₃) δ 7.30 (t, J=8.1 Hz, 1H), 7.22 (dd, J=8.3, 0.6 Hz, 1H), 7.14 (t, J_{HF}=53.4 Hz, 1H), 6.78 (d, J=7.6 Hz, 1H), 6.08 (s, 1H), 4.06 (s, 3H), 3.80 (m, 8H), 3.64 (m, 4H), 3.50 (m, 4H), 1.48 (s, 9H); Anal. Calcd. for C₂₆H₃₃F₂N₇O₄: C, 57.2; H, 6.1; N, 18.0. Found: 57.4; H, 6.1; N, 17.9%.

Treatment of the above carbamate with TFA in CH₂Cl₂ gave 2-(difluoromethyl)-4-methoxy-1-[6-(4-morpholinyl)-2-(1-piperazinyl)-4-pyrimidinyl]-1H-benzimidazole which was treated with chloromethanesulfonyl chloride as above. Chromatography on silica, eluting with CH₂Cl₂/EtOAc (9:1) gave (in 88% yield) 1-[2-{4-[(chloromethyl)sulfonyl]-1-piperazinyl}-6-(4-morpholinyl)-4-pyrimidinyl]-2-(difluoromethyl)-4-methoxy-1H-benzimidazole: mp (MeOH) 215-218° C.; ¹H NMR (CDCl₃) δ 7.31 (t, J=8.1 Hz, 1H), 7.20 (d, J=8.3 Hz, 1H), 7.10 (t, J_{HF}=53.3 Hz, 1H), 6.79 (d, J=7.9 Hz, 1H), 6.11 (s, 1H), 4.55 (s, 2H), 4.06 (s, 3H), 3.93 (m, 4H), 3.81 (m, 4H), 3.65 (m, 4H), 3.53 (m, 4H); Anal. Calcd. for C₂₂H₂₆ClF₂N₇O₄S: C, 47.35; H, 4.7; N, 17.6. Found: C, 47.6; H, 4.7; N, 17.9%.

### Example 17

### Synthesis of 1-[4-[1-(chloroacetyl)-4-piperidinyl]-6-(4-morpholinyl)-1,3,5-triazin-2-yl]-2-(difluoromethyl)-4-methoxy-1H-benzimidazole

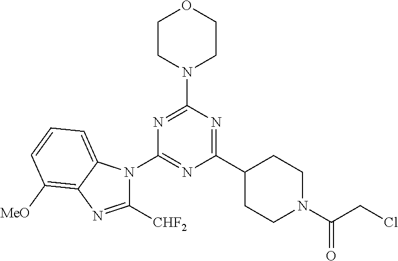

The compound was prepared according to Scheme 1 using Method A.

A mixture of 0.397 g (1 mmol) of 1-[4-chloro-6-(4-morpholinyl)-1,3,5-triazin-2-yl]-2-(difluoromethyl)-4-methoxy-1H-benzimidazole, 0.464 g (1.5 mmol) of tert-butyl 4-(4,4,5,5-tetramethyl-1,3,2-dioxaborolan-2-yl)-3,6-dihydro-1(2H)-pyridinecarboxylate, 56 mg PdCl₂(dppf)₂, and 8 mL of 2 M aq. Na₂CO₃ in 40 mL of dioxane was heated under reflux under nitrogen for 2 hrs. The dioxane was removed under vacuum and the residue was extracted in to CH₂Cl₂. Chromatography on silica, eluting with CH₂Cl₂/EtOAc (95:5) gave 0.51 g (94% yield) of tert-butyl 4-[4-[2-(difluoromethyl)-4-methoxy-1H-benzimidazol-1-yl]-6-(4-morpholinyl)-1,3,5-triazin-2-yl]-3,6-dihydro-1(2H)-pyridinecarboxylate: mp (MeOH) 223-225° C.; ¹H NMR (CDCl₃) δ 8.00 (dd, J=8.4, 0.6 Hz, 1H), 7.56 (t, J_{HF}=53.5 Hz, 1H), 7.39 (t, J=8.2 Hz, 1H), 7.38 (m, 1H), 6.85 (d, J=7.7 Hz, 1H), 4.23 (br d, J=3.0 Hz, 2H), 4.06 (s, 3H), 4.01 (m, 2H), 3.95 (m, 2H), 3.82 (m, 4H), 3.65 (t, J=5.7 Hz, 2H), 2.69 (m, 2H), 1.54-1.45 (m, 2H), 1.50 (s, 9H); Anal. Calcd. for C₂₆H₃₁F₂N₇O₄: C, 57.45; H, 5.75; N, 18.0. Found: C, 57.4; H, 5.9; N, 18.15%.

A solution of the above compound in a 1:1 mixture of MeOH and THF (100 mL) was hydrogenated over 100 mg of 5% Pd on carbon. After removal of the hydrogen, the mixture was heated under reflux in air for 2 hrs. The Pd on C was removed by filtration through celite, and the solvents were removed under vacuum. Recrystallization of the residue from methanol gave tert-butyl 4-[4-[2-(difluoromethyl)-4-methoxy-1H-benzimidazol-1-yl]-6-(4-morpholinyl)-1,3,5-triazin-2-yl]-1-piperidinecarboxylate: mp (MeOH) 177-179° C.; ¹H NMR (CDCl₃) δ 8.01 (dd, J=8.4, 0.7 Hz, 1H), 7.58 (t, J_{HF}=53.6 Hz, 1H), 7.38 (t, J=8.2 Hz, 1H), 6.84 (d, J=7.8 Hz, 1H), 4.22 (m, 2H), 4.05 (s, 3H), 3.99 (m, 2H), 3.94 (m, 2H), 3.81 (m, 4H), 2.94-2.78 (m, 3H), 2.05 (dd, J=13.0, 1.9 Hz, 2H), 1.81 (qd, J=12.7, 4.4 Hz, 2H), 1.49 (s, 9H); Anal. Calcd. for C₂₆H₃₃F₂N₇O₄: C, 57.2; H, 6.1; N, 18.0. Found: C, 57.4; H, 6.15; N, 18.1%.

Reaction of 0.13 g (0.24 mmol) of the above carbamate with TFA (5 mL) in CH₂Cl₂ (10 mL) gave 2-(difluoromethyl)-4-methoxy-1-[4-(4-morpholinyl)-6-(4-piperidinyl)-1,3,5-triazin-2-yl]-1H-benzimidazole which was treated with chloroacetyl chloride as before, to give a reaction product which was purified by chromatography on alumina, eluting with CH₂Cl₂/EtOAc (4:1), to give 73 mg (48% yield) of 1-[4-[1-(chloroacetyl)-4-piperidinyl]-6-(4-morpholinyl)-1,3,5-triazin-2-yl]-2-(difluoromethyl)-4-methoxy-1H-benzimidazole: mp (MeOH) 241-243° C.; ¹H NMR (CDCl₃) δ 7.99 (dd, J=8.4, 0.4 Hz, 1H), 7.55 (t, J_{HF}=53.6 Hz, 1H), 7.39 (t, J=8.3 Hz, 1H), 6.85 (d, J=8.0 Hz, 1H), 4.64 (br d, J=13.6 Hz, 2H), 4.12 (d, J=4.2 Hz, 2H), 4.06 (s, 3H), 4.01-3.92 (m, 5H), 3.82 (m, 4H), 3.31 (br t, J=11.8 Hz, 1H), 2.99-2.84 (m, 3H), 2.17 (br t, J=13.3 Hz, 2H), 2.01-1.82 (m, 2H); Anal. Calcd. for C₂₃H₂₆ClF₂N₇O₃: C, 52.9; H, 5.0; Cl, 6.8; N, 18.8. Found: C, 52.85; H, 5.0; Cl, 6.8; N, 18.7%.

### Example 18

### Synthesis of N-[1-(chloroacetyl)-4-piperidinyl]-4-[2-(difluoromethyl)-4-methoxy-1H-benzimidazol-1-yl]-6-(4-morpholinyl)-1,3,5-triazin-2-amine

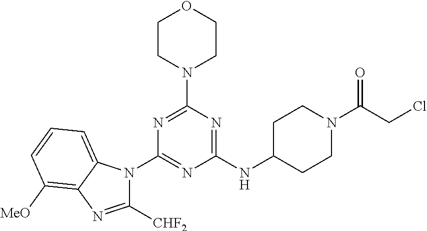

The compound was prepared according to Scheme 2 using Method B.

A stirred mixture of 1 g (5 mmol) of tert-butyl 4-piperidinylcarbamate and 1.3 g (10 mmol) of DIPEA in 25 mL of CH₂Cl₂ was cooled to 0° C. and 0.85 g (7.5 mmol) of chloroacetyl chloride was added. The solution was allowed to warm to room temperature overnight before being quenched with water, washed successively with aqueous acetic acid and NaHCO₃, and dried. Elution through a short column of alumina with CH₂Cl₂ gave 0.89 g (64% yield) of tert-butyl 1-(chloroacetyl)-4-piperidinylcarbamate: mp (CH₂Cl₂/hexanes) 102-103° C.; ¹H NMR (CDCl₃) δ 4.44 (br d, J=12.2 Hz, 1H), 4.06 (q, J=12.1 Hz, 2H), 3.82 (br d, J=12.4 Hz, 1H), 3.69 (m, 1H), 3.20 (br t, J=13.1 Hz, 1H), 2.82 (br t, J=11.4 Hz, 1H), 2.09-1.94 (m, 2H), 1.45 (s, 9H), 1.44-1.28 (m, 2H); Anal. Calcd. for C₁₂H₂₁ClN₂O₃: C, 52.1; H, 7.65; N, 10.1. Found: C, 52.0; H, 7.85; N, 10.2%.

A mixture of 0.55 g (2 mmol) of tert-butyl 1-(chloroacetyl)-4-piperidinylcarbamate and 5 g TFA in 20 mL CH₂Cl₂ was stirred at room temperature overnight and the resulting solution was evaporated to dryness, to give 1-(chloroacetyl)-4-piperidinamine trifluoroacetate as an oil: ¹H NMR (DMSO-d₆) δ 7.50 (m, exchangeable with D₂O, 3H), 4.41-4.28 (m, 3H), 3.86 (br d, J=13.7 Hz, 1H), 3.28 (m, 1H), 3.12 (t, J=12.3 Hz, 1H), 2.71 (t, J=12.1 Hz, 1H), 1.92 (br d, J=12.1 Hz, 2H), 1.52-1.44 (m, 1H), 1.39-1.27 (m, 1H).

A mixture of the above crude trifluoroacetate, 0.397 g (1 mmol) of 1-[4-chloro-6-(4-morpholinyl)-1,3,5-triazin-2-yl]-2-(difluoromethyl)-4-methoxy-1H-benzimidazole, and 0.52 g (4 mmol) of DIPEA in 50 mL of THF was stirred at room temperature for 5 days when water was added. The precipitate was collected, dried, and chromatographed on silica, eluting with CH₂Cl₂/EtOAc (1:1), to give 0.28 g (52% yield) of N-[1-(chloroacetyl)-4-piperidinyl]-4-[2-(difluoromethyl)-4-methoxy-1H-benzimidazol-1-yl]-6-(4-morpholinyl)-1,3,5-triazin-2-amine. mp (EtOH) 270-273° C.; ¹H NMR (CDCl₃) (rotamers; ratio ca. 3:2) δ 7.94 (dd, J=8.4, 0.6 Hz, 1H), 7.53 and 7.50 (2t, J_{HF}=53.6 Hz, 1H), 7.35 (t, J=7.9 Hz, 1H), 6.82 (t, J=6.8 Hz, 1H), 5.22 and 5.17 (2d, J=7.5 Hz, exchangeable with D₂O, 1H), 4.56 (m, 1H), 4.23-4.07 (m, 3H), 4.05 (s, 3H), 3.91-3.70 (m, 9H), 3.31 (t, J=11.5 Hz, 1H), 2.95 (m, 1H), 2.35-2.00 (m, 2H), 1.34-1.25 (m, 2H); Anal. Calcd. for C₂₃H₂₇ClF₂N₈O₃.0.2H₂O: C, 51.1; H, 5.1; N, 20.7. Found: C, 51.3; H, 5.1; N, 20.4%.

### Example 19

### Synthesis of N-[1-(chloroacetyl)-4-piperidinyl]-4-[2-(difluoromethyl)-4-methoxy-1H-benzimidazol-1-yl]-N-methyl-6-(4-morpholinyl)-1,3,5-triazin-2-amine

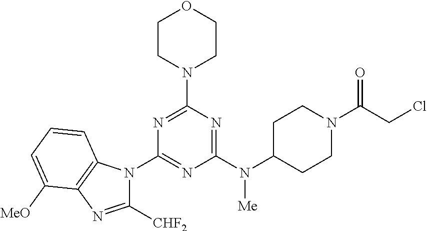

The compound was prepared according to Scheme 1 using Method A.

A mixture of 0.397 g (1 mmol) of 1-[4-chloro-6-(4-morpholinyl)-1,3,5-triazin-2-yl]-2-(difluoromethyl)-4-methoxy-1H-benzimidazole, 0.24 g (1.2 mmol) of tert-butyl 4-amino-1-piperidinecarboxylate and 0.194 g (1.5 mmol) of DIPEA in 25 mL of THF was stirred at room temperature overnight. Dilution with water and extraction with CH₂Cl₂, followed by chromatography on silica, eluting with CH₂Cl₂/EtOAc (4:1), gave 0.51 g (91% yield) of tert-butyl 4-{[4-[2-(difluoromethyl)-4-methoxy-1H-benzimidazol-1-yl]-6-(4-morpholinyl)-1,3,5-triazin-2-yl]amino}-1-piperidinecarboxylate: mp (hexanes/CH₂Cl₂) 142-145° C.; ¹H NMR (CDCl₃) (rotamer mixture; ratio ca. 3:2) δ 7.96 and 7.95 (2d, J=8.3 and 7.9 Hz, 1H), 7.54 and 7.52 (2t, J_{HF}=53.6 Hz, 1H), 7.34 (br t, J=8.1 Hz, 1H), 6.81 (t, J=6.9 Hz, 1H), 5.22 and 5.17 (2d, J=7.4 and 7.6 Hz, exchangeable with D₂O, 1H), 4.10 (m, 3H), 4.05 (s, 3H), 3.87 (m, 4H), 3.78 (m, 4H), 2.93 (t, J=12.1 Hz, 2H) 2.06 (m, 2H), 1.48 (s, 9H), 1.43 (m, 2H); Anal. Calcd. for C₂₆H₃₄F₂N₈O₄: C, 55.7; H, 6.1; N, 20.0. Found: C, 55.6; H, 6.2; N, 20.0%.

A solution of 0.30 g (5.4 mmol) of the above compound in 10 mL of DMF was treated sequentially with excess NaH and iodomethane at room temperature for 2 hrs. Dilution with water and workup in CH₂Cl₂, followed by chromatography on silica, eluting with CH₂Cl₂/EtOAc (4:1) gave 0.286 g (93% yield) of tert-butyl 4-[[4-[2-(difluoromethyl)-4-methoxy-1H-benzimidazol-1-yl]-6-(4-morpholinyl)-1,3,5-triazin-2-yl](methyl)amino]-1-piperidinecarboxylate: mp (MeOH/CH₂Cl₂) 200-202° C.; ¹H NMR (CDCl₃) (rotamer mixture; ratio ca. 3:2) δ 7.98 and 7.91 (2d, J=8.4 Hz, 1H), 7.57 and 7.47 (2t, J_{HF}=53.5 Hz, 1H), 7.34 (t, J=8.2 Hz, 1H), 6.81 (d, J=7.9 Hz, 1H), 4.82 and 4.70 (2m, 1H), 4.29 (m, 2H), 4.05 (s, 3H), 3.88 (m, 4H), 3.79 (m, 4H), 3.10 and 3.05 (2s, 3H), 2.84 (m, 2H), 1.73 (m, 4H), 1.49 (s, 9H); Anal. Calcd. for C₂₇H₃₆F₂N₈O₄: C, 56.4; H, 6.3; N, 19.5. Found: C, 56.6; H, 6.4; N, 19.6%.

Treatment of 0.173 g (0.3 mmol) of the above carbamate with TFA (5 mL) in CH₂Cl₂ (10 mL) at room temperature gave 0.143 g (100% yield) of crude 4-[2-(difluoromethyl)-4-methoxy-1H-benzimidazol-1-yl]-N-methyl-6-(4-morpholinyl)-N-(4-piperidinyl)-1,3,5-triazin-2-amine: ¹H NMR (CDCl₃) (rotamers; ratio ca. 3:2) δ 7.99 and 7.94 (2d, J=8.4 Hz, 1H), 7.59 and 7.52 (2t, J_{HF}=53.6 Hz, 1H), 7.34 (t, J=8.2 Hz, 1H), 6.81 (d, J=8.0 Hz, 1H), 4.80-4.63 (m, 1H), 4.06 (s, 3H), 3.88 (m, 4H), 3.79 (m, 4H), 3.25 (m, 2H), 3.13 and 3.09 (2s, 3H), 2.88-2.73 (m, 2H), 1.98-1.72 (m, 4H), 1.49 (s, 9H).

A stirred mixture of 0.143 g (0.3 mmol) of the above amine and 0.12 g (0.9 mmol) of DIPEA in 20 mL CH₂Cl₂ was cooled to 0° C. and treated with 51 mg (0.45 mmol) of chloroacetyl chloride. The mixture was allowed to warm to room temperature and was quenched with water after 2 hrs. After being washed successively with aqueous acetic acid and NaHCO₃ solutions, the solvent was dried and removed. Chromatography on silica, eluting with CH₂Cl₂/EtOAc (7:3), gave 0.146 g (88% yield) of N-[1-(chloroacetyl)-4-piperidinyl]-4-[2-(difluoromethyl)-4-methoxy-1H-benzimidazol-1-yl]-N-methyl-6-(4-morpholinyl)-1,3,5-triazin-2-amine: mp (MeOH) 211-213° C.; ¹H NMR (CDCl₃) (rotamers; ratio ca. 3:2) δ 7.97 and 7.89 (2d, J=8.4 Hz, 1H), 7.56 and 7.44 (2t, J_{HF}=53.5 Hz, 1H), 7.34 (t, J=8.2 Hz, 1H), 6.82 (d, J=8.0 Hz, 1H), 4.95 and 4.78 (2m, 2H), 4.17-3.99 (m, 3H), 4.05 (s, 3H), 3.89 (m, 4H), 3.79 (m, 4H), 3.28 (m, 1H), 3.12 and 3.05 (2s, 3H), 2.75 (m, 1H), 1.82 (m, 4H); Anal. Calcd. for C₂₄H₂₉ClF₂N₈O₃: C, 52.3; H, 5.3; N, 20.3. Found: C, 52.3; H, 5.2; N, 20.5%.

### Example 20

### Synthesis of N-[1-(chloroacetyl)-4-piperidinyl]-4-{2-(difluoromethyl)-4-[3-(dimethylamino)propoxy]-1H-benzimidazol-1-yl}-N-methyl-6-(4-morpholinyl)-1,3,5-triazin-2-amine

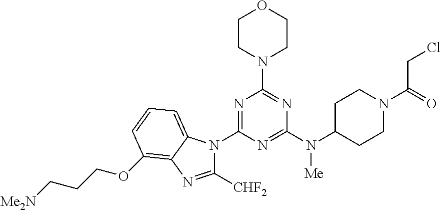

The compound was prepared according to Scheme 1 using Method A.

A mixture of 4-{[tert-Butyl(dimethyl)silyl]oxy}-1-[4-chloro-6-(4-morpholinyl)-1,3,5-triazin-2-yl]-2-(difluoromethyl)-1H-benzimidazole (see Example 5) (783 mg, 1.58 mmol), tert-butyl 4-(methylamino)-1-piperidinecarboxylate (337 mg, 1.58 mmol) and DIPEA (excess) in THF (12 mL) was stirred at room temperature for 20 hrs. The reaction mixture was diluted with water, extracted into CH₂Cl₂, dried (Na₂SO₄), and the solvent was removed. The resulting residue was chromatographed on silica, eluting with CH₂Cl₂/EtOAc (9:1), to give tert-butyl 4-[[4-[4-{[tert-butyl(dimethyl)silyl]oxy}-2-(difluoromethyl)-1H-benzimidazol-1-yl]-6-(4-morpholinyl)-1,3,5-triazin-2-yl](methyl)amino]-1-piperidinecarboxylate (892 mg, 84%) as a white solid: ¹H NMR (DMSO-d₆) (rotamers) δ 7.99 and 7.93 (2d, J=8.4 Hz, 1H), 7.74 and 7.70 (2t, J_{HF}=53.0 Hz, 1H), 7.38-7.32 (m, 1H), 6.86 (d, J=6.9 Hz, 1H), 4.74-4.63 (m, 1H), 4.16-4.07 (m, 2H), 3.80 (m, 4H), 3.69 (m, 4H), 3.06 and 3.03 (2s, 3H), 2.84 (m, 2H), 1.67 (m, 4H), 1.42 (s, 9H), 1.02 (s, 9H), 0.26 (s, 6H).

To a solution of the above compound (850 mg, 1.26 mmol) in THF (15 mL) was added TBAF (5 mL, 1 M solution in THF), and the reaction mixture was stirred at 20° C. for 30 min. The reaction solution was concentrated under vacuum at 20° C. and the residue was diluted with water. The resulting precipitate was filtered, washed with water, and dried to give tert-butyl 4-[[4-[2-(difluoromethyl)-4-hydroxy-1H-benzimidazol-1-yl]-6-(4-morpholinyl)-1,3,5-triazin-2-yl](methyl)amino]-1-piperidinecarboxylate (620 mg, 88%) as a white solid: ¹H NMR (DMSO-d₆) (rotamers) δ 10.24 (br s, exchangeable with D₂O, 1H), 7.81 and 7.75 (2d, J=8.3, 8.2 Hz, 1H), 7.75 and 7.71 (2t, J_{HF}=53.0 Hz, 1H), 7.29-7.23 (m, 1H), 6.76 (dd, J=7.9, 0.6 Hz, 1H), 4.76-4.60 (m, 1H), 4.15-4.08 (m, 2H), 3.80 (m, 4H), 3.70 (m, 4H), 3.06 and 3.03 (2s, 3H), 2.82 (m, 2H), 1.67 (m, 4H), 1.42 (s, 9H).

To a mixture of the above phenol (531 mg, 0.95 mmol), and K₂CO₃ (2 g) in DMF (6 mL) was added 3-bromo-1-propanol (0.4 mL, 4.75 mmol). The reaction mixture was stirred at 20° C. for 20 hrs. The reaction mixture was diluted with water, and the resulting precipitate was filtered, washed with water, and dried. Recrystallization from CH₂Cl₂/hexanes gave tert-butyl 4-[[4-[2-(difluoromethyl)-4-(3-hydroxypropoxy)-1H-benzimidazol-1-yl]-6-(4-morpholinyl)-1,3,5-triazin-2-yl](methyl)amino]-1-piperidinecarboxylate (537 mg, 91%) as a white solid: ¹H NMR (DMSO-d₆) (rotamers) δ 7.96 and 7.89 (2d, J=8.4 Hz, 1H), 7.74 and 7.70 (2t, J_{HF}=53.3, 52.9 Hz, 1H), 7.42-7.36 (m, 1H), 6.95 (d, J=8.1 Hz, 1H), 4.73-4.63 (m, 1H), 4.56 (t, J=5.1 Hz, exchangeable with D₂O, 1H), 4.29 (t, J=6.4 Hz, 2H), 4.14-4.07 (m, 2H), 3.81 (m, 4H), 3.69 (m, 4H) 3.65-3.61 (m, 2H), 3.06 and 3.03 (2s, 3H), 2.82 (m, 2H), 2.01-1.94 (m, 2H), 1.67 (m, 4H), 1.42 (s, 9H).

To a cooled (ice/salt) mixture of the above alcohol (503 mg; 0.81 mmol) and Et₃N (0.5 mL, excess) in THF (10 mL) was added methanesulfonyl chloride (0.5 mL) and the resulting mixture was stirred at this temperature for 30 min. A solution of 40% aq. dimethylamine (5 mL) was added, and the reaction mixture was allowed to warm to room temperature. After 20 hrs, the mixture was diluted with water, and the resulting precipitate was collected and dried. Chromatography on neutral alumina, eluting with CH₂Cl₂/MeOH (49:1), gave 523 mg (100% yield) of tert-butyl 4-[[4-{2-(difluoromethyl)-4-[3-(dimethylamino)propoxy]-1H-benzimidazol-1-yl}-6-(4-morpholinyl)-1,3,5-triazin-2-yl](methyl)amino]-1-piperidinecarboxylate as a sticky solid: ¹H NMR (DMSO-d₆) (rotamers) δ 7.95 and 7.89 (2d, J=8.4 Hz, 1H), 7.70 and 7.67 (2t, J_{HF}=52.9 Hz, 1H), 7.42-7.35 (m, 1H), 6.93 (d, J=8.1 Hz, 1H), 4.73-4.63 (m, 1H), 4.25 (t, J=6.5 Hz, 2H), 4.15-4.07 (m, 2H), 3.80 (m, 4H), 3.70 (m, 4H), 3.06 and 3.03 (2 s, 3H), 2.83 (s, 2H), 2.42 (t, J=7.1 Hz, 2H), 2.16 (s, 6H), 1.20-1.92 (m, 2H), 1.62 (m, 4H), 1.42 (s, 9H).

To a solution of the above carbamate (530 mg, 0.82 mmol) in CH₂Cl₂ (10 mL) was added TFA (10 mL) and the reaction mixture was stirred at 20° C. for 1 hr. The solvents were removed under vacuum at 20° C. and the residue was diluted with water (100 mL), and basified with aq. NH₃. The resulting precipitate was filtered, washed with water, and dried, to give 390 mg (87% yield) of 4-{2-(difluoromethyl)-4-[3-(dimethylamino)propoxy]-1H-benzimidazol-1-yl}-N-methyl-6-(4-morpholinyl)-N-(4-piperidinyl)-1,3,5-triazin-2-amine as a white solid: ¹H NMR (DMSO-d₆) (rotamers) δ 7.96 and 7.88 (2d, J=8.3 Hz, 1H), 7.72 and 7.59 (2t, J_{HF}=53.0, 52.8 Hz, 1H), 7.41-7.36 (m, 1H), 6.95 (d, J=8.0 Hz, 1H), 4.81-4.70 (m, 1H), 4.26 (t, J=6.4 Hz, 2H), 3.82 (m, 4H), 3.70 (m, 4H), 3.08 and 4.04 (2s, 3H), 3.28 (m, 2H), 3.06-2.87 (m, 2H), 2.55-2.51 (m, 2H), 2.26 (s, 6H), 2.03-1.79 (m, 6H).

To a suspension of the above amine (254 mg, 0.46 mmol) and powdered K₂CO₃ (3 g) in CH₂Cl₂ (5 mL) at 0° C. was added chloroacetyl chloride (1 mL) and the reaction mixture was allowed to warm to room temperature. After 1 hr, the mixture was diluted with H₂O (50 mL) and the organic layer was separated and dried (Na₂SO₄). The solution was absorbed on to column of neutral alumina, and the column was then eluted with CH₂Cl₂/MeOH (49:1). The combined fractions containing the product were acidified with 1.25 M HCl in MeOH (2 mL), and the solution was evaporated to dryness. Recrystallization of the residue from CH₂Cl₂/EtOAc/hexanes gave 85 mg (28% yield) of N-[1-(chloroacetyl)-4-piperidinyl]-4-{2-(difluoromethyl)-4-[3-(dimethylamino)propoxy]-2,3-dihydro-1H-benzimidazol-1-yl}-N-methyl-6-(4-morpholinyl)-1,3,5-triazin-2-amine hydrochloride as a white solid: mp 128° C. (dec); ¹H NMR (DMSO-d₆) (rotamers) δ 10.08 (br s, exchangeable with D₂O, 1H), 8.00 and 7.94 (d, J=8.4 Hz, 1H), 7.75 and 7.73 (2t, J_{HF}=52.9 Hz, 1H), 7.45-7.39 (m, 1H), 6.99 (d, J=8.1 Hz, 1H), 4.81-4.72 (m, 1H), 4.56-4.48 (m, 1H), 4.42 (br s, 2H), 4.34 (t, J=6.0 Hz, 2H), 4.01-3.95 (m, 1H), 3.81 (m, 4H), 3.70 (m, 4H), 3.28 (m, 2H), 3.22-3.11 (m, 1H), 3.06 and 3.03 (2s, 3H), 2.82 (s, 6H), 2.82-2.66 (m, 1H), 2.28-2.21 (m, 2H), 1.89-1.66 (m, 4H); Anal. Calcd. for C₂₈H₃₉Cl₂F₂N₉O₃. 1.2H₂O: C, 49.4; H, 6.1; Cl, 10.4; N, 18.5. Found: C, 49.8; H, 6.1; Cl, 10.2; N, 18.0%.

### Example 21

### Synthesis of N-{1-[(chloromethyl)sulfonyl]-4-piperidinyl}-4-[2-(difluoromethyl)-4-methoxy-1H-benzimidazol-1-yl]-N-methyl-6-(4-morpholinyl)-1,3,5-triazin-2-amine

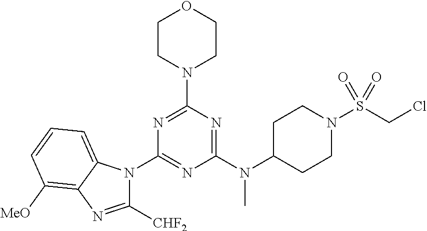

Reaction of 4-[2-(difluoromethyl)-4-methoxy-1H-benzimidazol-1-yl]-N-methyl-6-(4-morpholinyl)-N-(4-piperidinyl)-1,3,5-triazin-2-amine (Example 19) with chloromethanesulphonyl chloride and powdered K₂CO₃ in CH₂Cl₂ gave N-{1-[(chloromethyl)sulfonyl]-4-piperidinyl}-4-[2-(difluoromethyl)-4-methoxy-1H-benzimidazol-1-yl]-N-methyl-6-(4-morpholinyl)-1,3,5-triazin-2-amine in 19% yield: mp (CH₂Cl₂/MeOH) 212-215° C.; ¹H NMR (DMSO-d₆) (rotamers) δ 7.97 and 7.90 (2d, J=8.4 Hz, 1H), 7.74 and 7.70 (2t, J_{HF}=52.9, 52.8 Hz, 1H), 7.44-7.39 (m, 1H), 7.44-7.39 (m, 1H), 6.96 (d, J=8.1 Hz 1H), 5.15 and 5.11 (2s, 2H), 4.73-4.66 (m, 2H), 3.98 (s, 3H), 3.91-3.81 (m, 6H), 3.70 (m, 4H) 3.18-3.05 (m, 2H), 3.09 and 3.05 (2s, 3H), 1.90-1.78 (m, 4H), Anal. Calcd. for C₂₃H₂₉ClF₂N₈O₄S: C, 47.1, H, 5.0; N, 19.1. Found: C, 47.2; H, 5.3; N, 19.2%.

### Example 22

### Synthesis of 2-chloro-N-{1-[4-[2-(difluoromethyl)-4-methoxy-1H-benzimidazol-1-yl]-6-(4-morpholinyl)-1,3,5-triazin-2-yl]-4-piperidinyl}acetamide

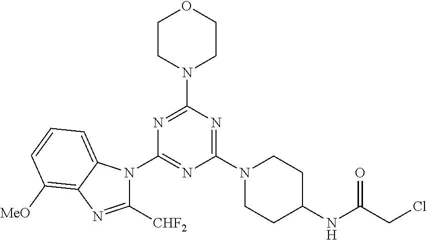

The compound was prepared according to Scheme 1 using Method A.

A mixture of 0.992 g (2.5 mmol) of 1-[4-chloro-6-(4-morpholinyl)-1,3,5-triazin-2-yl]-2-(difluoromethyl)-4-methoxy-1H-benzimidazole, 1.00 g (5 mmol) tert-butyl 4-piperidinylcarbamate, and 0.65 g (5 mmol) DIPEA in 100 mL of THF was stirred at room temperature for 30 min, before the solution was concentrated, and diluted with water containing 1 mL of acetic acid. The resulting solid was collected, washed with water, and dried to give 1.38 g (98%) of tert-butyl 1-[4-[2-(difluoromethyl)-4-methoxy-1H-benzimidazol-1-yl]-6-(4-morpholinyl)-1,3,5-triazin-2-yl]-4-piperidinylcarbamate: mp (MeOH) 208-209° C.; ¹H NMR (CDCl₃) δ 7.88 (d, J=8.1 Hz, 1H), 7.48 (t, J_{HF}=53.6 Hz, 1H), 7.34 (t, J=8.2 Hz, 1H), 6.81 (d, J=7.9 Hz, 1H), 4.66 (br d, J=13.4 Hz, 2H), 4.46 (m, exchangeable with D₂O, 1H), 4.04 (s, 3H), 3.87 (m, 4H), 3.78 (m, 5H), 3.12 (m, 2H), 2.07 (br d, J=14.0 Hz, 2H), 1.46 (s, 9H), 1.45-1.33 (m, 2H); Anal. Calcd. for C₂₆H₃₄F₂N₈O₄: C, 55.7; H, 6.1; N, 20.0. Found: C, 55.85; H, 6.1; N, 20.1%.

Treatment of 0.28 g (0.5 mmol) of the above carbamate with TFA (5 mL) in CH₂Cl₂ (10 mL) gave a quantitative yield of crude 1-[4-[2-(difluoromethyl)-4-methoxy-1H-benzimidazol-1-yl]-6-(4-morpholinyl)-1,3,5-triazin-2-yl]-4-piperidinamine. ¹H NMR (CDCl₃) δ 7.90 (d, J=8.4 Hz, 1H), 7.51 (t, J_{HF}=53.6 Hz, 1H), 7.34 (t, J=8.2 Hz, 1H), 6.81 (d, J=8.0 Hz, 1H), 4.66 (br d, J=13.1 Hz, 2H), 4.05 (s, 3H), 3.88 (m, 4H), 3.78 (m, 4H), 3.15-2.96 (m, 3H), 1.94 (m, 2H), 1.39-1.25 (m, 2H).

Treatment of the preceding amine with chloroacetyl chloride as before gave a crude product which was recrystallized from methanol to give 0.165 g (61% yield) of (2-chloro-N-{1-[4-[2-(difluoromethyl)-4-methoxy-1H-benzimidazol-1-yl]-6-(4-morpholinyl)-1,3,5-triazin-2-yl]-4-piperidinyl}acetamide: mp 252-254° C.; ¹H NMR (CDCl₃) δ 7.88 (d, J=8.1 Hz, 1H), 7.48 (t, J_{HF}=53.6 Hz, 1H), 7.35 (t, J=8.2 Hz, 1H), 6.81 (d, J=7.9 Hz, 1H), 6.47 (d, J=7.9 Hz, exchangeable with D₂O, 1H), 4.72 (br d, J=13.6 Hz, 2H), 4.13 (m, 1H), 4.06 (s, 2H), 4.05 (s, 3H), 3.88 (m, 4H), 3.78 (m, 4H), 3.15 (m, 2H), 2.09 (br dd, J=12.6, 2.7 Hz, 2H), 1.56-1.44 (m, 2H); Anal. Calcd. for C₂₃H₂₇ClF₂N₈O₃: C, 51.45; H, 5.1; N, 20.9. Found: C, 51.45; H, 5.0; N, 20.9%.

### Example 23

### Synthesis of 2-chloro-N-{1-[4-[2-(difluoromethyl)-4-methoxy-1H-benzimidazol-1-yl]-6-(4-morpholinyl)-1,3,5-triazin-2-yl]-4-piperidinyl}-N-methylacetamide

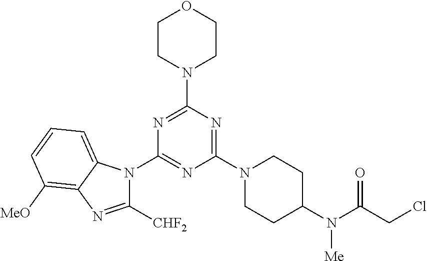

The compound was prepared according to Scheme 1 using Method A.

Methylation of 0.31 g (5.5 mmol) of tert-butyl 1-[4-[2-(difluoromethyl)-4-methoxy-1H-benzimidazol-1-yl]-6-(4-morpholinyl)-1,3,5-triazin-2-yl]-4-piperidinylcarbamate (Example 22) with 3 equivalents of 60% NaH dispersion in oil (27.5 mg, 16.5 mmol) and iodomethane in DMF (35 mL) gave 0.31 g (98% yield) of tert-butyl 1-[4-[2-(difluoromethyl)-4-methoxy-1H-benzimidazol-1-yl]-6-(4-morpholinyl)-1,3,5-triazin-2-yl]-4-piperidinyl(methyl)carbamate: mp (MeOH) 182-183° C.; ¹H NMR (CDCl₃) δ 7.89 (d, J=8.0 Hz, 1H), 7.50 (t, J_{HF}=52.8 Hz, 1H), 7.34 (t, J=7.5 Hz, 1H), 6.81 (d, J=7.9 Hz, 1H), 4.90 (br d, J=12.2 Hz, 2H), 4.27 (m, 1H), 4.05 (s, 3H), 3.88 (m, 4H), 3.78 (m, 4H), 2.96 (m, 2H), 2.72 (s, 3H), 1.78 (m, 2H), 1.71-1.59 (m, 2H), 1.48 (s, 9H); Anal. Calcd. for C₂₇H₃₆F₂N₈O₄: C, 56.4; H, 6.3; N, 19.5. Found: C, 56.4; H, 6.2; N, 19.6%.

Treatment of 0.173 g (0.3 mmol) of the above carbamate with TFA (5 mL) in CH₂Cl₂ (10 mL) gave a quantitative yield of 1-[4-[2-(difluoromethyl)-4-methoxy-1H-benzimidazol-1-yl]-6-(4-morpholinyl)-1,3,5-triazin-2-yl]-N-methyl-4-piperidinamine. ¹H NMR (CDCl₃) δ 7.90 (dd, J=8.4, 0.7 Hz, 1H), 7.51 (t, J_{HF}=53.6 Hz, 1H), 7.34 (t, J=8.2 Hz, 1H), 6.80 (d, J=7.6 Hz, 1H), 4.63 (br d, J=13.2 Hz, 2H), 4.04 (s, 3H), 3.88 (m, 4H), 3.78 (m, 4H), 3.12 (m, 2H), 2.70 (m, 1H), 2.48 (s, 3H), 2.03 (m, 2H), 1.41-1.30 (m, 2H).

Treatment of the above amine with chloroacetyl chloride as before, followed by chromatography on silica, eluting with CH₂Cl₂/EtOAc (7:3) gave 0.20 g (81% yield) of 2-chloro-N-{1-[4-[2-(difluoromethyl)-4-methoxy-1H-benzimidazol-1-yl]-6-(4-morpholinyl)-1,3,5-triazin-2-yl]-4-piperidinyl}-N-methylacetamide: mp (MeOH) 206-208° C.; ¹H NMR (CDCl₃) δ 7.89 (d, J=8.4 Hz, 1H), 7.49 (t, J_{HF}=53.6 Hz, 1H), 7.35 (t, J=8.2 Hz, 1H), 6.81 (d, J=8.0 Hz, 1H), 4.94 (m, 2H), 4.74 (m, 1H), 4.09 (s, 2H), 4.05 (s, 3H), 3.88 (m, 4H), 3.78 (m, 4H), 3.07-2.83 (m, 2H), 2.93 (s, 3H), 1.93-1.57 (m, 4H); Anal. Calcd. for C₂₄H₂₉ClF₂N₈O₃: C, 52.3; H, 5.3; N, 20.3. Found: C, 52.4; H, 5.3; N, 20.3%.

### Example 24

### Synthesis of 2-chloro-N-{1-[4-{2-(difluoromethyl)-4-[3-(dimethylamino)propoxy]-1H-benzimidazol-1-yl}-6-(4-morpholinyl)-1,3,5-triazin-2-yl]-4-piperidinyl}-N-methylacetamide

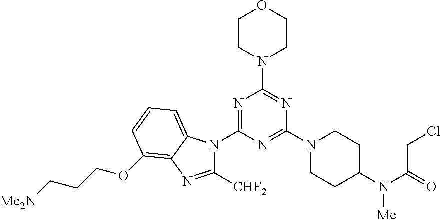

The compound was prepared according to Scheme 1 using Method A.

A mixture of 1.11 g (2.2 mmol) of 4-(tert-butyldimethylsilyloxy)-1-[4-chloro-6-(4-morpholinyl)-1,3,5-triazin-2-yl]-2-(difluoromethyl)-1H-benzimidazole (Example 5), 0.48 g (2.3 mmol) of tert-butyl methyl(4-piperidinyl)carbamate, and 0.6 g DIPEA in 20 mL THF was stirred at room temperature for 1 hr. After dilution with water containing 1 mL acetic acid, the product was extracted in to EtOAc, washed successively with water and aq. NaHCO₃, and dried. Chromatography on silica, eluting with CH₂Cl₂/hexane (19:1) gave 1.20 g (81% yield) of tert-butyl 1-[4-[4-{[tert-butyl(dimethyl)silyl]oxy}-2-(difluoromethyl)-1H-benzimidazol-1-yl]-6-(4-morpholinyl)-1,3,5-triazin-2-yl]-4-piperidinyl(methyl)carbamate as an oil: ¹H NMR (CDCl₃) δ 7.90 (d, J=8.0 Hz, 1H), 7.47 (t, J_{HF}=53.6 Hz, 1H), 7.26 (t, J=8.1 Hz, 1H), 6.83 (d, J=7.5 Hz, 1H), 4.89 (m, 2H), 4.27 (m, 1H), 3.87 (m, 4H), 3.78 (m, 4H), 2.95 (m, 2H), 2.72 (s, 3H), 1.70-1.59 (m, 4H), 1.48 (s, 9H), 1.05 (s, 9H), 0.30 (s, 6H).

Reaction of the above silyl ether in 20 mL of THF with 1 M TBAF in THF (3.65 mL, 2 equiv), followed by quenching with water and recrystallization from aq. MeOH gave 0.79 g (79% yield) of tert-butyl 1-[4-[2-(difluoromethyl)-4-hydroxy-1H-benzimidazol-1-yl]-6-(4-morpholinyl)-1,3,5-triazin-2-yl]-4-piperidinyl(methyl)carbamate: mp 220-222° C.; ¹H NMR (DMSO-d₆) δ 10.20 (s, exchangeable with D₂O, 1H), 7.74 (dd, J=8.3, 0.6 Hz, 1H), 7.70 (t, J_{HF}=53.0 Hz, 1H), 7.26 (t, J=8.1 Hz, 1H), 6.76 (dd, J=7.9, 0.7 Hz, 1H), 4.78 (m, 2H), 4.10 (m, 1H), 3.80 (m, 4H), 3.69 (m, 4H), 2.99 (m, 2H), 2.66 (s, 3H), 1.67 (m, 4H), 1.41 (s, 9H); Anal. Calcd. for C₂₆H₃₄lF₂N₈O₄: C, 55.7; H, 6.1; N, 20.0. Found: C, 55.9; H, 6.0; N, 20.0%.

A mixture of 0.448 g (0.8 mmol) of the above phenol, 0.34 g (2.4 mmol) of 3-bromo-1-propanol and 138 mg (1.0 mmol) of K₂CO₃ in DMF (20 mL) was stirred at room temperature overnight. Dilution with water gave an oily solid which was extracted with CH₂Cl₂ and dried. Chromatography on silica, eluting with CH₂Cl₂/EtOAc (1:1), gave 0.484 g (98% yield) of tert-butyl 1-[4-[2-(difluoromethyl)-4-(3-hydroxypropoxy)-1H-benzimidazol-1-yl]-6-(4-morpholinyl)-1,3,5-triazin-2-yl]-4-piperidinyl(methyl)carbamate: ¹H NMR (DMSO-d₆) δ7.88 (d, J=8.0 Hz, 1H), 7.69 (t, J_{HF}=52.9 Hz, 1H), 7.38 (t, J=8.2 Hz, 1H), 6.94 (d, J=7.8 Hz, 1H), 4.82-4.73 (m, 2H), 4.56 (t, J=5.1 Hz, 1H), 4.28 (t, J=6.4 Hz, 2H), 4.09 (m, 1H), 3.80 (m, 4H), 3.69 (m, 4H), 3.63 (dd, J=11.5, 6.1 Hz, 2H), 3.00 (m, 2H), 2.66 (s, 3H), 1.97 (pentet, J=6.3 Hz, 2H), 1.67 (m, 4H), 1.41 (s, 9H).

The above alcohol was combined with 0.23 g (2 mmol) of methanesulphonyl chloride and 0.2 g (2 mmol) of Et₃N in THF at 0° C., and after 1 hr the mixture was diluted with 5 mL of 40% aq. Me₂NH. The resulting mixture was stirred at room temperature for 24 hrs and the solvent was removed. The residue was diluted with water and extracted with CH₂Cl₂. Chromatography on alumina eluting with CH₂Cl₂/EtOAc (9:1) gave 0.51 g (100% yield) of tert-butyl 1-[4-{2-(difluoromethyl)-4-[3-(dimethylamino)propoxy]-1H-benzimidazol-1-yl}-6-(4-morpholinyl)-1,3,5-triazin-2-yl]-4-piperidinyl(methyl)carbamate: ¹H NMR (CDCl₃) δ 7.88 (d, J=8.3 Hz, 1H), 7.49 (t, J_{HF}=53.6 Hz, 1H), 7.32 (t, J=8.2 Hz, 1H), 6.84 (d, J=8.0 Hz, 1H), 4.90 (m, 2H), 4.32 (t, J=6.8 Hz, 2H), 4.28 (m, 1H), 3.88 (m, 4H), 3.78 (m, 4H), 2.96 (m, 2H), 2.72 (s, 3H), 2.51 (t, J=7.1 Hz, 2H), 2.26 (s, 6H), 2.12 (pentet, J=7.0 Hz, 2H), 1.78 (m, 2H), 1.68 (m, 2H), 1.48 (s, 9H).

Treatment of the above carbamate with TFA (5 mL) in CH₂Cl₂ (10 mL) gave 0.42 g (98% yield) of 1-[4-{2-(difluoromethyl)-4-[3-(dimethylamino)propoxy]-1H-benzimidazol-1-yl}-6-(4-morpholinyl)-1,3,5-triazin-2-yl]-N-methyl-4-piperidinamine. ¹H NMR (CDCl₃) δ 7.89 (d, J=8.3 Hz, 1H), 7.51 (t, J_{HF}=53.6 Hz, 1H), 7.31 (t, J=8.2 Hz, 1H), 6.84 (d, J=8.0 Hz, 1H), 4.63 (m, 2H), 4.32 (t, J=6.8 Hz, 2H), 3.87 (m, 4H), 3.78 (m, 4H), 3.13 (m, 2H), 2.69 (m, 1H), 2.52 (t, J=7.2 Hz, 2H), 2.48 (s, 3H), 2.27 (s, 6H), 2.12 (pentet, J=7.0 Hz, 2H), 2.00 (m, 2H), 1.35 (dt, J=14.0, 4.1 Hz, 2H).

The above amine was reacted with chloroacetyl chloride and K₂CO₃ in CH₂Cl₂ at 0° C., and the product was purified by chromatography on alumina, eluting with EtOAc/MeOH (99:1). The combined product containing fractions were acidified with HCl (1.25 M) in MeOH, and the solvents were removed under vacuum. Recrystallization of the residue from EtOH/EtOAc gave 0.33 g (65% yield) of 2-chloro-N-{1-[4-{2-(difluoromethyl)-4-[3-(dimethylamino)-propoxy]-1H-benzimidazol-1-yl}-6-(4-morpholinyl)-1,3,5-triazin-2-yl]-4-piperidinyl}-N-methylacetamide hydrochloride: mp 245° C. dec.; ¹H NMR (DMSO-d₆) 10.18 (m, exchangeable with D₂O, 1H), 7.92 (d, J=8.4 Hz, 1H), 7.71 (t, J_{HF}=52.8 Hz, 1H), 7.41 (t, J=8.2 Hz, 1H), 6.98 (d, J=8.1 Hz, 1H), 4.85-4.72 (m, 2H), 4.57 (m, 0.5H), 4.50 and 4.38 (2s, 2H), 4.33 (t, J=6.1 Hz, 2H), 4.00 (m, 0.5H), 3.81 (m, 4H), 3.69 (m, 4H), 3.28 (m, 2H), 3.05 (m, 2H), 2.84 and 2.70 (2s, 3H), 2.81 (s, 6H), 2.24 (dt, J=12.3, 6.1 Hz, 2H), 1.80-1.60 (m, 2H); Anal. Calcd. for C₂₈H₃₉O₂F₂N₉O₃H₂O: C, 49.7; H, 6.1; Cl, 10.5; N, 18.6. Found: 49.95; H, 6.0; Cl, 10.6; N, 18.6%.

### Example 25

### Synthesis of 2-chloro-N-{(3R)-1-[4-[2-(difluoromethyl)-4-methoxy-1H-benzimidazol-1-yl]-6-(4-morpholinyl)-1,3,5-triazin-2-yl]pyrrolidinyl}acetamide

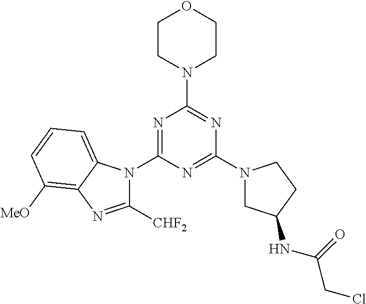

The compound was prepared according to Scheme 1 using Method A.

A mixture of 1-[4-chloro-6-(4-morpholinyl)-1,3,5-triazin-2-yl]-2-(difluoromethyl)-4-methoxy-1H-benzimidazole (420 mg, 1.06 mmol), tert-butyl (3R)-pyrrolidinylcarbamate (0.24 g, 1.27 mmol), and DIPEA (0.3 mL, 1.6 mmol) in THF (25 mL) was stirred at 20° C. for 20 hrs. The reaction mixture was diluted with water (100 mL), and the resulting precipitate was filtered, washed with water, and recrystallized from CH₂Cl₂/hexanes, to give 554 mg (96% yield) of tert-butyl (3R)-1-[4-[2-(difluoromethyl)-4-methoxy-1H-benzimidazol-1-yl]-6-(4-morpholinyl)-1,3,5-triazin-2-yl]pyrrolidinylcarbamate: mp 151-153° C.; ¹H NMR (DMSO-d₆) δ 8.01 and 7.98 (2d, J=8.3, 8.9 Hz, 1H), 7.77 and 7.74 (2t, J_{HF}=53.0 Hz, 1H) 7.40 and 7.39 (2t, J=8.2 Hz, 1H), 7.20 (br s, exchangeable with D₂O, 1H), 6.94 (d, J=8.1 Hz, 1H), 4.13 (br, 1H), 3.92 (s, 3H), 3.79 (m, 4H), 3.78-3.37 (m, 2H), 3.68 (m, 4H), 3.28 (m, 2H), 2.20-2.08 and 1.95-1.84 (2m, 2H), 1.40 (s, 9H).

A solution of tert-butyl (3R)-1-[4-[2-(difluoromethyl)-4-methoxy-1H-benzimidazol-1-yl]-6-(4-morpholinyl)-1,3,5-triazin-2-yl]pyrrolidinylcarbamate (200 mg, 0.36 mmol) in CH₂Cl₂ (10 mL) was treated with TFA (5 mL) and stirred for 3 hrs. The solvent and excess TFA was evaporated at 20° C. under vacuum, and the resulting residue was diluted with H₂O (50 mL), and basified with aq NH₃. The resulting precipitate was filtered, washed with water and dried, to give 143 mg (89% yield) of (3R)-1-[4-[2-(difluoromethyl)-4-methoxy-1H-benzimidazol-1-yl]-6-(4-morpholinyl)-1,3,5-triazin-2-yl]-3-pyrrolidinamine The amine was dissolved in CH₂Cl₂ (10 mL) containing a suspension of powdered K₂CO₃ (3 g), and chloroacetyl chloride (1 mL) was added at 0° C. After stirred for 3 hrs at 20° C., the reaction mixture was diluted with water (20 mL), and the organic layer was separated and dried. Chromatography on silica eluting with CH₂Cl₂/MeOH (47:3) containing 1% aq. NH₃ gave 129 mg (77% yield) of 2-chloro-N-{(3R)-1-[4-[2-(difluoromethyl)-4-methoxy-1H-benzimidazol-1-yl]-6-(4-morpholinyl)-1,3,5-triazin-2-yl]pyrrolidinyl}acetamide as a white solid: mp (CH₂Cl₂/MeOH) 270-275° C.; ¹H NMR (DMSO-d₆) (rotamers) δ 8.54 (d, J=6.4 Hz, 1H), 8.02 and 7.99 (2d, J=8.2 Hz, 1H), 7.78 and 7.75 (2t, J_{HF}=53.0 Hz, 1H), 7.41 (t, J=8.2 Hz, 1H), 6.95 (d, J=8.1 Hz, 1H), 4.44-4.35 (m, 1H), 4.05 (s, 2H), 3.97 (s, 3), 3.87-3.63 (m, 10H), 3.55 and 3.46 (2dd, J=11.6, 4.1, and 12.0, 4.2 Hz, 2H), 2.26-2.14 and 2.01-1.88 (2m, 2H); Anal. Calcd. for C₂₂H₂₅ClF₂N₈O₃: C, 50.5; H, 4.8; N, 21.4. Found: C, 50.7; H, 5.0; N, 21.2%.

### Example 26

### Synthesis of 2-chloro-N-{(3S)-1-[4-[2-(difluoromethyl)-4-methoxy-1H-benzimidazol-1-yl]-6-(4-morpholinyl)-1,3,5-triazin-2-yl]pyrrolidinyl}acetamide

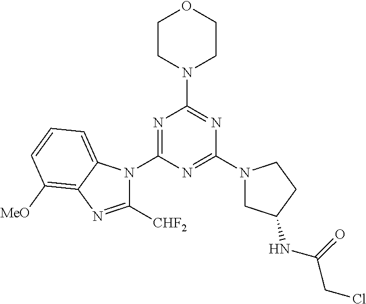

The compound was prepared according to Scheme 1 using Method A.

Similarly, reaction of 1-[4-chloro-6-(4-morpholinyl)-1,3,5-triazin-2-yl]-2-(difluoromethyl)-4-methoxy-1H-benzimidazole with tert-butyl (3S)-pyrrolidinylcarbamate as in Example 25 gave tert-butyl (3S)-1-[4-[2-(difluoromethyl)-4-methoxy-1H-benzimidazol-1-yl]-6-(4-morpholinyl)-1,3,5-triazin-2-yl]pyrrolidinylcarbamate in 91% yield as a white solid: mp (CH₂Cl₂/MeOH) 292° C. dec.; ¹H NMR (DMSO-d₆) δ 8.01 and 7.98 (2d, J=8.2, 8.4 Hz, 1H), 7.77 and 7.75 (2t, J_{HF}=53.0 Hz, 1H), 7.40 (2t, J=8.2 Hz, 1H), 7.21 (br, exchangeable with D₂O, 1H), 6.95 (d, J=8.0 Hz, 1H), 4.15-411 (m, 1H), 3.97 (s, 3H), 3.79-3.37 (m, 12H), 3.00-2.08 and 1.95-1.05 (2m, 2H), 1.40 (s, 9H).

Deprotection of the carbamate with TFA in CH₂Cl₂ gave (3S)-1-[4-[2-(difluoromethyl)-4-methoxy-1H-benzimidazol-1-yl]-6-(4-morpholinyl)-1,3,5-triazin-2-yl]-3-pyrrolidinamine in 95% yield. Reaction of the amine with chloroacetyl chloride as in Example 25 gave 2-chloro-N-{(3S)-1-[4-[2-(difluoromethyl)-4-methoxy-1H-benzimidazol-1-yl]-6-(4-morpholinyl)-1,3,5-triazin-2-yl]pyrrolidinyl}acetamide in 95% yield: ¹H NMR (DMSO-d₆) (rotamers) δ 8.54 (d, J=6.4 Hz, exchangeable with D₂O, 1H), 8.02 and 7.99 (2d, J=8.1, 8.3 Hz, 1H), 7.78 and 7.75 (2t, J_{HF}=53.1 Hz, 1H), 7.40 (t, J=8.2 Hz, 1H), 6.95 (d, J=8.1 Hz, 1H), 4.44-4.33 (m, 1H), 4.04 (s, 2H), 3.98 (s, 3H), 3.87-3.44 (m, 12H), 2.26-2.14 and 2.01-1.88 (2m, 2H); Anal. Calcd. for C₂₂H₂₅ClF₂N₈O₃: C, 50.5; H, 4.8; N, 21.4. Found: C, 50.5, H, 4.9; N, 21.4%.

### Example 27

### Synthesis of 2-chloro-N-{(3R)-1-[4-[2-(difluoromethyl)-4-methoxy-1H-benzimidazol-1-yl]-6-(4-morpholinyl)-1,3,5-triazin-2-yl]pyrrolidinyl}-N-methylacetamide

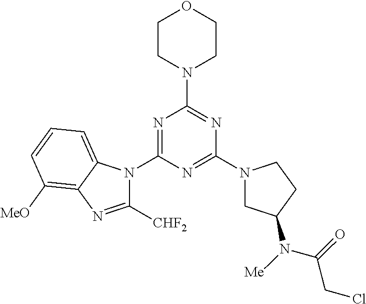

The compound was prepared according to Scheme 1 using Method A.

To a solution of 322 mg (0.59 mmol) of tert-butyl (3R)-1-[4-[2-(difluoromethyl)-4-methoxy-1H-benzimidazol-1-yl]-6-(4-morpholinyl)-1,3,5-triazin-2-yl]pyrrolidinylcarbamate (Example 25) in dry DMF (5 mL) at 0° C. was added NaH (42 mg, 1.77 mmol). The reaction mixture was stirred for 30 min at this temp and then MeI (1 mL) was added. The resulting mixture was allowed to warm to 20° C. over 3 hrs, and was then carefully diluted with water. The precipitate was filtered, washed with water, and dried. Recrystallization from CH₂Cl₂/hexanes gave 290 mg (88% yield) of tert-butyl (3R)-1-[4-[2-(difluoromethyl)-4-methoxy-1H-benzimidazol-1-yl]-6-(4-morpholinyl)-1,3,5-triazin-2-yl]pyrrolidinyl(methyl)carbamate: mp 180-182° C.; ¹H NMR (DMSO-d₆) δ 8.00 (t, J=8.4 Hz, 1H), 7.77 (t, J_{HF}=53.0 Hz, 1H), 7.40 (t, J=8.2 Hz, 1H), 6.95 (d, J=7.9 Hz, 1H), 4.76-4.66 (m, 1H), 3.98 (s, 3H), 3.87-3.75 (m, 6H), 3.69 (m, 4H), 3.62-3.38 (m, 2H), 2.77 (d, J=3.5 Hz, 3H), 2.16-2.07 (m, 2H), 1.43 (s, 9H).

The above carbamate was deprotected with TFA in CH₂Cl₂ to give (3R)-1-[4-[2-(difluoromethyl)-4-methoxy-1H-benzimidazol-1-yl]-6-(4-morpholinyl)-1,3,5-triazin-2-yl]-N-methyl-3-pyrrolidinamine in 100% yield: ¹H NMR (DMSO-d₆) (rotamers) δ 8.02 and 8.00 (2d, J=8.1 Hz, 1H), 7.77 (t, J_{HF}=53.0 Hz, 1H), 7.40 (t, J=8.2 Hz, 1H), 6.95 (d, J=8.1 Hz, 1H), 3.98 (s, 3H), 3.81-3.80 (m, 4H), 3.79-3.38 (m, 8H), 2.39 (s, 3H), 1.19-2.08 and 1.96-1.86 (2m, 2H).

Reaction of the amine with chloroacetyl chloride in similar manner as previous examples gave 2-chloro-N-{(3R)-1-[4-[2-(difluoromethyl)-4-methoxy-1H-benzimidazol-1-yl]-6-(4-morpholinyl)-1,3,5-triazin-2-yl]pyrrolidinyl}-N-methylacetamide in 89% yield: mp (CH₂Cl₂/hexanes) 231-233° C.; ¹H NMR (DMSO-d₆) (rotamers) δ 8.02 and 7.98 (2d, J=8.3 Hz, 1H), 7.78 (br t, J_{HF}=53.0 Hz, 1H), 7.40 (t, J=8.5 Hz, 1H), 6.95 (d, J=8.8 Hz, 1H), 5.14-5.03 and 4.71-4.52 (2m, 1H), 4.54-4.52 and 4.44-4.42 (2m, 2H), 3.98 and 3.97 (2s, 3H), 3.90-3.42 (m, 12H), 2.95 and 2.82 (2s, 3H), 2.23-2.09 (m, 2H); Anal. Calcd. for C₂₃H₂₇ClF₂N₈O₃: 51.5; H, 5.1; N, 20.9. Found: C, 51.3; H, 5.1; N, 20.8%.

### Example 28

### Synthesis of 2-chloro-N-{(3S)-1-[4-[2-(difluoromethyl)-4-methoxy-1H-benzimidazol-1-yl]-6-(4-morpholinyl)-1,3,5-triazin-2-yl]pyrrolidinyl}-N-methylacetamide

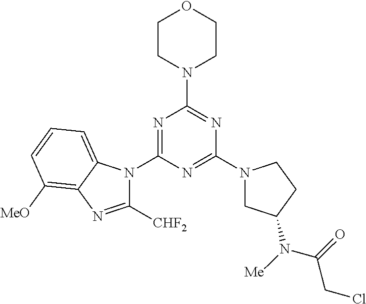

The compound was prepared according to Scheme 1 using Method A.

Similarly to Example 27, tert-butyl (3S)-1-[4-[2-(difluoromethyl)-4-methoxy-1H-benzimidazol-1-yl]-6-(4-morpholinyl)-1,3,5-triazin-2-yl]pyrrolidinyl(methyl)carbamate was prepared by methylation of tert-butyl (3S)-1-[4-[2-(difluoromethyl)-4-methoxy-1H-benzimidazol-1-yl]-6-(4-morpholinyl)-1,3,5-triazin-2-yl]pyrrolidinylcarbamate (Example 26) in 88% yield: mp (CH₂Cl₂/hexanes) 182-185° C.; ¹H NMR (DMSO-d₆) (rotamers) δ 8.00 and 7.99 (2d, J=8.1 Hz, 1H), 7.61 and 7.60 (2t, J_{HF}=53.6 Hz, 1H), 7.35 and 7.34 (2t, J=8.1, 8.2 Hz, 1H), 5.56 (d, J=8.0 Hz, 1H), 4.87 (m, 1H), 4.05 (s, 3H), 3.95-3.82 (m, 10H), 3.64-3.43 (m, 2H), 2.84 (s, 3H), 2.23-2.06 (m, 2H), 1.49 (s, 9H).

Deprotection of the above carbamate with TFA in CH₂Cl₂ gave (3S)-1-[4-[2-(difluoromethyl)-4-methoxy-1H-benzimidazol-1-yl]-6-(4-morpholinyl)-1,3,5-triazin-2-yl]-N-methyl-3-pyrrolidinamine in 100% yield: ¹H NMR (DMSO-d₆) (rotamers) δ 8.08 (br, exchangeable with D₂O, 1H), 8.01 and 7.99 (2d, J=8.5 Hz, 1H), 7.77 (t, J_{HF}=53.0 Hz, 1H), 7.41 (t, J=8.2 Hz, 1H), 6.96 (d, J=8.1 Hz, 1H), 3.98 (s, 3H), 3.96-3.56 (m, 13H), 3.64 (s, 3H), 2.40-2.29 and 2.21-2.10 (2m, 2H).

Reaction of the amine with chloroacetyl chloride gave 2-chloro-N-{(3S)-1-[4-[2-(difluoromethyl)-4-methoxy-1H-benzimidazol-1-yl]-6-(4-morpholinyl)-1,3,5-triazin-2-yl]pyrrolidinyl}-N-methylacetamide in 83% yield: mp (CH₂Cl₂/hexanes) 230-232° C.; ¹H NMR (DMSO-d₆) (rotamers) δ 8.02 and 7.98 (2d, J=8.3, 8.1 Hz, 1H), 7.78 (br t, J_{HF}=53.0 Hz, 1H), 7.41 (t, J=8.2 Hz, 1H), 6.95 (d, J=8.1 Hz, 1H), 5.18-4.99 and 4.71-4.66 (2m, 1H), 4.53-4.51 and 4.44-4.42 (2m, 2H), 3.98 (s, 3H), 3.91-3.39 (m, 12H), 2.95 and 2.82 (2s, 3H), 2.23-2.11 (m, 2H); Anal. Calcd. for C₂₃H₂₇ClF₂N₈O₃: 51.5; H, 5.1; N, 20.9. Found: C, 51.2; H, 5.1; N, 20.6%.

### Example 29

### Synthesis of N-[(3R)-1-(chloroacetyl)pyrrolidinyl]-4-[2-(difluoromethyl)-4-methoxy-1H-benzimidazol-1-yl]-6-(4-morpholinyl)-1,3,5-triazin-2-amine

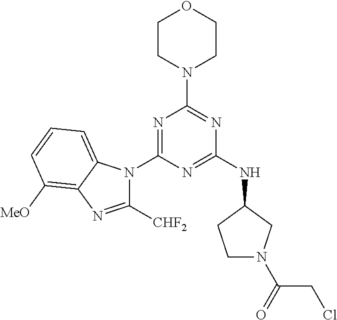

The compound was prepared according to Scheme 1 using Method A.

Reaction of 1-[4-chloro-6-(4-morpholinyl)-1,3,5-triazin-2-yl]-2-(difluoromethyl)-4-methoxy-1H-benzimidazole with tert-butyl (3R)-3-amino-1-pyrrolidinecarboxylate as in previous examples gave tert-butyl (3R)-3-{[4-[2-(difluoromethyl)-4-methoxy-1H-benzimidazol-1-yl]-6-(4-morpholinyl)-1,3,5-triazin-2-yl]amino}-1-pyrrolidinecarboxylate in 93% yield: mp (CH₂Cl₂/hexanes) 224-226° C.; ¹H NMR (DMSO-d₆) (rotamers) δ 8.16 (d, J=6.12 Hz, exchangeable with D₂O, 1H), 8.11 and 7.95 (dd, J=8.2, 8.4 Hz, 1H), 7.87 (t, J_{HF}=53.3 Hz, 1H), 7.42-7.35 (m, 1H), 6.95 and 6.94 (2d, J=8.0 Hz, 1H), 4.49 (m, 1H), 3.97 (2s, 3H), 3.78 (m, 4H), 3.70 (m, 4H), 3.65-3.22 (m, 4H), 2.17-2.13 and 1.99-1.92 (2m, 2H), 1.42 (br s, 9H).

Deprotection of the above carbamate with TFA in CH₂Cl₂ gave 4-[2-(difluoromethyl)-4-methoxy-1H-benzimidazol-1-yl]-6-(4-morpholinyl)-N-[(3S)-pyrrolidinyl]-1,3,5-triazin-2-amine which was reacted with chloroacetyl chloride to give N-[(3R)-1-(chloroacetyl)pyrrolidinyl]-4-[2-(difluoromethyl)-4-methoxy-1H-benzimidazol-1-yl]-6-(4-morpholinyl)-1,3,5-triazin-2-amine in 33% yield: mp (CH₂Cl₂/hexanes) 208-210° C.; ¹H NMR (DMSO-d₆) (rotamers) δ 8.20, 8.18, and 8.15 (3d, J=6.7, 6.5, 6.7 Hz, exchangeable with D₂O, 1H), 8.10 and 7.96 (2d, J=8.3, 8.5 Hz, 1H), 7.86, 7.75 and 7.74 (3t, J_{HF}=53.0 Hz, 1H), 7.43-7.36 (m, 1H), 6.97-6.93 (m, 1H), 4.59-4.46 (m, 1H), 4.36-4.22 (m, 2H), 3.97 (br s, 3H), 3.88-3.39 (m, 12H), 2.29-1.93 (m, 2H); Anal. Calcd. for C₂₂H₂₅ClF₂N₈O₃: C, 50.5; H, 4.8; N, 21.4. Found: C, 50.9; H, 5.0; N, 20.9%.

### Example 30

### Synthesis of N-[(3S)-1-(chloroacetyl)pyrrolidinyl]-4-[2-(difluoromethyl)-4-methoxy-1H-benzimidazol-1-yl]-6-(4-morpholinyl)-1,3,5-triazin-2-amine

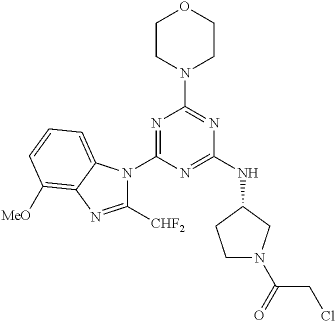

The compound was prepared according to Scheme 1 using Method A.

Similarly to Example 29, reaction of 1-[4-chloro-6-(4-morpholinyl)-1,3,5-triazin-2-yl]-2-(difluoromethyl)-4-methoxy-1H-benzimidazole with tert-butyl (3S)-3-amino-1-pyrrolidinecarboxylate gave tert-butyl (3S)-3-[[4-[2-(difluoromethyl)-4-methoxy-1H-benzimidazol-1-yl]-6-(4-morpholinyl)-1,3,5-triazin-2-yl](methyl)amino]-1-pyrrolidinecarboxylate in 91% yield: mp (CH₂Cl₂/hexanes) 225-228° C.; ¹H NMR (DMSO-d₆) (rotamers) δ 8.16 (d, J=6.2 Hz, exchangeable with D₂O, 1H), 8.11 and 7.96 (2d, J=8.2, 8.4 Hz, 1H), 7.87 (t, J_{HF}=53.3 Hz, 1H), 7.42-7.36 (m, 1H), 6.96 and 6.94 (2d, J=5.0 Hz, 1H), 4.60-4.46 (m, 1H), 3.98 (s, 3H), 3.79 (m, 4H), 3.70 (m, 4H), 3.62-3.55 and 4.36-3.40 (2m, 2H), 3.34-3.23 (m, 2H), 2.19-2.11 and 1.98-1.91 (2m, 2H), 1.41 (s, 9H).

The above carbamate was deprotected with TFA in CH₂Cl₂ to give 4-[2-(difluoromethyl)-4-methoxy-1H-benzimidazol-1-yl]-N-methyl-6-(4-morpholinyl)-N-[(3S)-pyrrolidinyl]-1,3,5-triazin-2-amine in 72% yield. Reaction of the amine with chloacetyl chloride as before gave N-[(3S)-1-(chloroacetyl)pyrrolidinyl]-4-[2-(difluoromethyl)-4-methoxy-1H-benzimidazol-1-yl]-N-methyl-6-(4-morpholinyl)-1,3,5-triazin-2-amine in 51% yield: mp (CH₂Cl₂/MeOH) 208-211° C.; ¹H NMR (DMSO-d₆) (rotomers) δ 8.20, 8.18, and 8.15 (3d, J=6.6, 6.5, 6.7 Hz, exchangeable with D₂O, 1H), 8.10 and 8.00 (2d, J=8.4 Hz, 1H) 7.86, 7.75 and 7.74 (3t, J_{HF}=53.0 Hz, 1H), 7.43-7.36 (m, 1H), 6.97-6.93 (m, 1H), 4.60-4.66 (m, 1H) 4.33-4.29 (m, 2H), 3.97 (2s, 3H), 3.78 (m, 4H), 3.69 (m, 4H), 3.65-3.37 (m, 2H), 2.29-2.20, 2.18-2.11, and 2.09-1.95 (3m, 2H); Anal. Calcd. for C₂₂H₂₅ClF₂N₈O₃: C, 50.5; H, 4.8; N, 21.4. Found: C, 50.55; H, 5.0; N, 21.2%.

### Example 31

### Synthesis of N-[(3R)-1-(chloro acetyl)pyrrolidinyl]-4-[2-(difluoromethyl)-4-methoxy-1H-benzimidazol-1-yl]-N-methyl-6-(4-morpholinyl)-1,3,5-triazin-2-amine

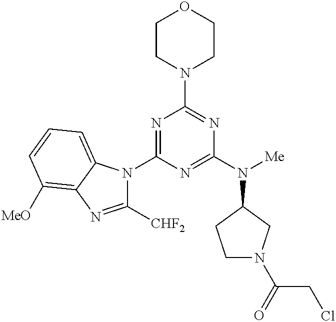

The compound was prepared according to Scheme 1 using Method A.

Methylation of tert-butyl (3R)-3-{[4-[2-(difluoromethyl)-4-methoxy-1H-benzimidazol-1-yl]-6-(4-morpholinyl)-1,3,5-triazin-2-yl]amino}-1-pyrrolidinecarboxylate (Example 29) with NaH and MeI in DMF as before gave tert-butyl (3R)-3-[[4-[2-(difluoromethyl)-4-methoxy-1H-benzimidazol-1-yl]-6-(4-morpholinyl)-1,3,5-triazin-2-yl](methyl)amino]-1-pyrrolidinecarboxylate in 96% yield: ¹H NMR (DMSO-d₆) δ 7.97-7.58 (m, 2H), 7.40 (t, J=8.2 Hz, 1H), 6.95 (d, J=7.8 Hz, 1H), 5.34-5.25 (m, 1H), 3.08 (s, 3H), 3.81 (m, 4H), 3.71-3.70 (m, 4H), 3.55-3.45 (m, 2H), 3.28 (m, 2H), 3.11-3.08 (m, 3H), 2.14-2.08 (m, 2H), 1.42 (s, 9H).

Deprotection of the carbamate with TFA in CH₂Cl₂ gave 4-[2-(difluoromethyl)-4-methoxy-1H-benzimidazol-1-yl]-N-methyl-6-(4-morpholinyl)-N-[(3R)-pyrrolidinyl]-1,3,5-triazin-2-amine in 100% yield: ¹H NMR (DMSO-d₆) δ 7.97-7.58 (m, 2H), 7.41 (t, J=8.2 Hz, 1H), 6.95 (d, J=7.8 Hz, 1H), 5.33-5.31 (m, 1H), 3.98 (s, 3H), 3.81-3.10 (m, 4H), 3.71-3.70 (m, 4H), 3.43-3.07 (m, 5H), 2.97-2.89 (m, 2H), 2.10-1.97 and 1.88-1.79 (2m, 2H).

Reaction of the amine with chloroacetyl chloride as before gave N-[(3R)-1-(chloroacetyl)pyrrolidinyl]-4-[2-(difluoromethyl)-4-methoxy-1H-benzimidazol-1-yl]-N-methyl-6-(4-morpholinyl)-1,3,5-triazin-2-amine in 85% yield: mp (CH₂Cl₂/hexanes) 219-221° C.; ¹H NMR (DMSO-d₆) (rotomers) δ 7.97-7.59 (m, 2H), 7.43-7.39 (m, 1H), 6.95 (d, J=8.1 Hz, 1H), 5.44-5.37 and 5.33-5.30 (2m, 1H), 4.39-4.32 (m, 2H), 3.98 (s, 3H), 3.82 (m, 4H), 3.77-3.26 (m, 8H), 3.13-3.10 (m, 3H), 2.24-2.18 and 2.15-2.09 (2m, 2H); Anal. Calcd. for C₂₃H₂₇ClF₂N₈O₃: C, 51.5, H, 5.1: N, 20.9. Found: C, 51.7; H, 5.1; 21.0%.

### Example 32

### Synthesis of N-[(3S)-1-(chloroacetyl)pyrrolidinyl]-4-[2-(difluoromethyl)-4-methoxy-1H-benzimidazol-1-yl]-N-methyl-6-(4-morpholinyl)-1,3,5-triazin-2-amine

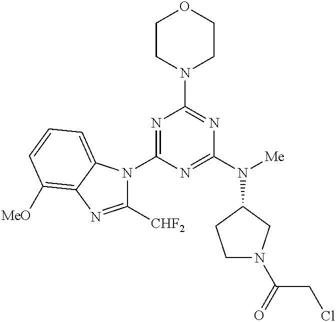

The compound was prepared according to Scheme 1 using Method A.

Similar methylation of tert-butyl (3S)-3-[[4-[2-(difluoromethyl)-4-methoxy-1H-benzimidazol-1-yl]-6-(4-morpholinyl)-1,3,5-triazin-2-yl] (methyl)amino]-1-pyrrolidinecarboxylate (Example 30) gave tert-butyl (3S)-3-[[4-[2-(difluoromethyl)-4-methoxy-1H-benzimidazol-1-yl]-6-(4-morpholinyl)-1,3,5-triazin-2-yl] (methyl)amino]-1-pyrrolidinecarboxylate in 94% yield: ¹H NMR (DMSO-d₆) (rotamers) δ 7.97-7.58 (m, 2H), 7.40 (t, J=8.2 Hz, 1H), 6.95 (d, J=7.8 Hz, 1H), 5.34-5.28 (m, 1H), 3.98 (s, 3H), 3.82 (m, 4H), 3.71-3.70 (m, 4H), 3.55-3.45 (m, 2H), 3.28 (m, 2H), 3.11-3.08 (m, 3H), 3.14-2.08 (m, 2H).

Deprotection of the above carbamate with TFA in CH₂Cl₂ gave 4-[2-(difluoromethyl)-4-methoxy-1H-benzimidazol-1-yl]-N-methyl-6-(4-morpholinyl)-N-[(3S)-pyrrolidinyl]-1,3,5-triazin-2-amine in 100% yield: ¹H NMR (DMSO-d₆) δ 8.04-7.55 (m, 2H), 7.40 (t, J=8.2 Hz, 1H), 6.95 (d, J=8.0 Hz, 1H), 5.29 (br, 1H), 3.98 (s, 3H), 3.81 (m, 4H), 3.70 (m, 4H), 4.00-2.99 (m, 5H), 2.89-2.81 (m, 2H), 2.06-1.98 and 1.82-1.73 (2m, 2H).

Reaction of the amine with chloroacetyl chloride gave N-[(3S)-1-(chloro acetyl)pyrrolidinyl]-4-[2-(difluoromethyl)-4-methoxy-1H-benzimidazol-1-yl]-N-methyl-6-(4-morpholinyl)-1,3,5-triazin-2-amine in 93% yield: mp (CH₂Cl₂/hexanes) 219-221° C.; ¹H NMR (DMSO-d₆) δ 7.97-7.58 (m, 2H), 7.41 (t, J=8.2 Hz, 1H), 7.40 (t, J=8.2 Hz, 1H), 6.95 (d, J=8.1 Hz, 1H), 5.44-5.37 and 5.33-5.29 (2m, 1H), 4.9, 4.35, 4.34 and 4.32 (4s, 2H), 3.81-3.34 (m, 12H), 3.18-3.11 (m, 3H), 2.24-2.18 and 2.15-2.09 (2m, 2H); Anal. Calcd. for C, 51.4; H, 5.1; N, 20.9. Found: C, 51.6; H, 5.2; N, 20.9%.

### Example 33

### Synthesis of 2-chloro-N-{(3R)-1-[4-[2-(difluoromethyl)-4-methoxy-1H-benzimidazol-1-yl]-6-(4-morpholinyl)-1,3,5-triazin-2-yl]piperidinyl}acetamide

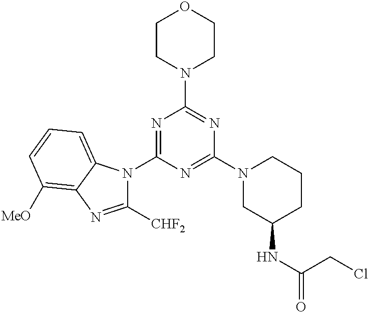

The compound was prepared according to Scheme 1 using Method A.

Reaction of 1-[4-chloro-6-(4-morpholinyl)-1,3,5-triazin-2-yl]-2-(difluoromethyl)-4-methoxy-1H-benzimidazole with tert-butyl (3R)-piperidinylcarbamate gave tert-butyl (3R)-1-[4-[2-(difluoromethyl)-4-methoxy-1H-benzimidazol-1-yl]-6-(4-morpholinyl)-1,3,5-triazin-2-yl]piperidinylcarbamate in 94% yield: mp (CH₂Cl₂/hexanes) 115-118° C.; ¹H NMR (DMSO-d₆) (rotamers) δ 8.00 and 7.89 (2d, J=8.1, 8.4 Hz, 1H), 7.72 and 7.69 (t, J_{HF}=52.6, 52.7 Hz, 1H), 7.43-7.36 (m, 1H), 6.95 (d, J=7.9 Hz, 1H), 6.95 (br, exchangeable with D₂O, 1H), 4.53-4.43, 4.37-4.31 and 4.21-4.13 (3m, 1H), 3.97 (s, 3H), 3.79 (m, 4H), 3.69 (m, 4H), 3.42-3.36 (m, 2H), 3.16-3.10 and 3.02-2.96 (2m, 2H) 1.88-1.79 (m, 2H), 1.55-1.40 (m, 2H), 1.40 and 1.38 (2s, 9H).

Deprotection of the above carbamate with TFA in CH₂Cl₂ gave (3R)-1-[4-[2-(difluoromethyl)-4-methoxy-1H-benzimidazol-1-yl]-6-(4-morpholinyl)-1,3,5-triazin-2-yl]-3-piperidinamine in 96% yield: ¹H NMR (DMSO-d₆) δ 7.88 (d, J=8.2 Hz, 1H), 7.72 and 7.68 (t, J_{HF}=53.4, 53.0 Hz, 1H), 7.41 (t, J=8.2 Hz, 1H), 7.41 (br, exchangeable with D₂O, 2H), 6.96 (d, J=8.2 Hz, 1H), 4.49-4.38 (m, 1H), 4.22-4.14 and 3.90 (2m, 2H), 3.98 (s, 3H), 3.81 (m, 4H), 3.70 (m, 4H), 3.94-3.40 (m, 2H), 2.03-2.00 and 1.81 (2m, 2H), 1.66-1.54 (m, 2H).

Reaction of the crude amine with chloroacetyl chloride gave 2-chloro-N-{(3R)-1-[4-[2-(difluoromethyl)-4-methoxy-1H-benzimidazol-1-yl]-6-(4-morpholinyl)-1,3,5-triazin-2-yl]piperidinyl}acetamide in 63% yield: mp (CH₂Cl₂/hexanes) 231-233° C.; ¹H NMR (DMSO-d₆) (rotamers) δ 8.26 and 8.17 (2d, J=7.2, 7.1 Hz, exchangeable with D₂O, 1H), 7.94 (2, J=8.3, 8.4 Hz, 1H), 7.69 (t, J_{HF}=52.9 Hz, 1H), 7.43-7.37 (m, 1H), 6.94 (d, J=8.1 Hz, 1H), 4.27-4.24 and 4.12-4.07 (2m, 2H), 4.12-4.05, 4.01 (ABq, J=12.8 Hz, 2H), 3.98 (s, 3H), 3.79-3.69 (m, 9H), 3.48-3.34 (m, 2H), 1.93-1.88 and 1.82-1.80 (2m, 2H) 1.64-1.53 (m, 2H); Anal. Calcd. for C₂₃H₂₇ClF₂N₈O₃: C, 51.5; H, 5.1; N, 20.9. Found: C, 51.7; H, 5.1; N, 20.8%.

### Example 34

### Synthesis of 2-chloro-N-{(3S)-1-[4-[2-(difluoromethyl)-4-methoxy-1H-benzimidazol-1-yl]-6-(4-morpholinyl)-1,3,5-triazin-2-yl]piperidinyl}acetamide

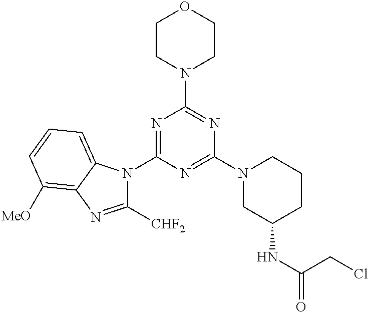

The compound was prepared according to Scheme 1 using Method A.

Similarly, reaction of 1-[4-chloro-6-(4-morpholinyl)-1,3,5-triazin-2-yl]-2-(difluoromethyl)-4-methoxy-1H-benzimidazole and tert-butyl (3S)-piperidinylcarbamate gave tert-butyl (3S)-1-[4-[2-(difluoromethyl)-4-methoxy-1H-benzimidazol-1-yl]-6-(4-morpholinyl)-1,3,5-triazin-2-yl]piperidinylcarbamate in 100% yield: mp (CH₂Cl₂/hexanes) 119-122° C.; ¹H NMR (DMSO-d₆) (rotamers) δ 8.00 and 7.89 (2d, J=8.4, 8.3 Hz, 1H), 7.72 and 7.69 (2t, J_{HF}=52.9 Hz, 1H), 7.43-7.36 (m, 1H), 6.95 (d, J=7.9 Hz, 1H), 6.96 (br, exchangeable with D₂O, 1H), 4.51-4.80, 4.37-4.34, and 4.21-4.14 (3m, 1H), 3.97 (s, H), 3.79 (m, 4H), 3.69 (m, 4H), 3.42-3.39 and 3.28 (2m, 2H), 3.17-3.10 and 3.02-2.96 (2m, 2H), 1.88-1.74 (m, 2H), 1.53-1.44 (m, 2H), 1.40 and 1.38 (2s, 9H).

Deprotection of the above carbamate with TFA in CH₂Cl₂ gave (3S)-1-[4-[2-(difluoromethyl)-4-methoxy-1H-benzimidazol-1-yl]-6-(4-morpholinyl)-1,3,5-triazin-2-yl]-3-piperidinamine in 100% yield: ¹H NMR (DMSO-d₆) (rotamers) δ 7.88 (d, J=8.3 Hz, 1H), 7.72 and 7.68 (2t, J_{HF}=53.0 Hz, 1H), 7.41 (t, J=8.2 Hz, 1H), 6.96 (d, J=8.1 Hz, 1H), 6.71 (br, exchangeable with D₂O, 2H), 4.52-4.35 (m, 1H), 4.25-4.16 and 3.10 (2m, 2H), 3.98 (s, 3H), 3.81 (m, 4H), 3.70 (m, 4H), 3.28 (m, 2H), 1.99 and 1.81 (2m, 2H), 1.55 (m, 2H).

Reaction of the amine with chloroacetyl chloride as before gave 2-chloro-N-{(3S)-1-[4-[2-(difluoromethyl)-4-methoxy-1H-benzimidazol-1-yl]-6-(4-morpholinyl)-1,3,5-triazin-2-yl]piperidinyl}acetamide in 80% yield; mp (CH₂Cl₂/hexanes) 227-229° C.; ¹H NMR (DMSO-d₆) (rotamers) δ 8.26 and 8.17 (2d, J=7.3, 7.1 Hz, exchangeable with D₂O, 1H), 7.94 and 7.90 (2d, J=8.4 Hz, 1H), 7.69 (t, J_{HF}=52.9 Hz, 1H), 7.43-7.37 (m, 1H), 6.94 (d, J=8.0 Hz, 1H), 4.27-4.24 and 4.12-4.00 (2m, 2H), 4.05 and 4.01 (ABq, J=12.8, 12.9 Hz, 2H), 3.97 (s, 3H), 3.79-3.69 (m, 9H), 3.48-3.37 (m, 2H), 1.92-1.88 and 1.83-1.80 (2m, 2H), 1.64-1.53 (m, 2H); Anal. Calcd. for C₂₃H₂₇ClF₂N₈O₃: C, 51.5; H, 5.1; N, 20.9. Found: C, 51.5; H, 5.1; N, 20.8%.

### Example 35

### Synthesis of 2-chloro-N-{(3R)-1-[4-[2-(difluoromethyl)-4-methoxy-1H-benzimidazol-1-yl]-6-(4-morpholinyl)-1,3,5-triazin-2-yl]piperidinyl}-N-methylacetamide

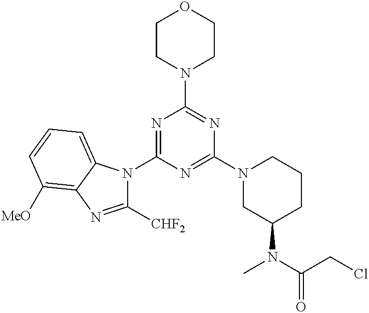

The compound was prepared according to Scheme 1 using Method A.

Methylation of tert-butyl (3R)-1-[4-[2-(difluoromethyl)-4-methoxy-1H-benzimidazol-1-yl]-6-(4-morpholinyl)-1,3,5-triazin-2-yl]piperidinylcarbamate (Example 33) with NaH and MeI in DMF gave tert-butyl (3R)-1-[4-[2-(difluoromethyl)-4-methoxy-1H-benzimidazol-1-yl]-6-(4-morpholinyl)-1,3,5-triazin-2-yl]piperidinyl(methyl)carbamate in 72% yield: ¹H NMR (DMSO-d₆) (rotamers) δ 7.89 (d, J=8.3 Hz, 1H), 7.69 and 7.66 (2t, J_{HF}=52.9, 52.8 Hz, 1H), 7.43-7.34 (m, 1H), 6.94 (d, J=8.0 Hz, 1H), 4.71 and 4.56 (m, 2H), 3.97 (s, 3H), 3.80-3.69 (m, 9H), 3.07-2.83 (m, 2H), 2.79 and 2.78 (2s, 3H), 1.88-1.80 (m, 3H), 1.52-1.25 (m, 1H), 1.41 (s, 9H); Anal. Calcd. for C₂₇H₃₆F₂N₈O₄: C, 56.7; H, 6.3; N, 19.5. Found: C, 56.7; H, 6.4; N, 19.7%.

Deprotection of the above carbamate with TFA in CH₂Cl₂ gave (3S)-1-[4-[2-(difluoromethyl)-4-methoxy-1H-benzimidazol-1-yl]-6-(4-morpholinyl)-1,3,5-triazin-2-yl]-N-methyl-3-piperidinamine ¹H NMR (DMSO-d₆) (rotamers) δ 7.94 and 7.89 (2d, J=8.3, 8.4 Hz, 1H), 7.71 and 7.68 (2t, J_{HF}=52.9 Hz, 1H), 7.42-7.36 (m, 1H), 6.94 (d, J=7.9 Hz, 1H), 4.52-4.49, 4.34-4.30 and 4.15-4.10 (m, 2H), 3.97 (s, 3H), 3.79 (m, 4H), 3.69 (m, 4H), 3.43-3.17 and 2.99-2.94 (2m, 3H), 2.45 (s, 3H), 1.94-1.90 and 1.76-1.74 (2m, 2H), 1.46-1.36 (m, 2H).

Reaction of the above crude amine with chloroacetyl chloride gave 2-chloro-N-{(3R)-1-[4-[2-(difluoromethyl)-4-methoxy-1H-benzimidazol-1-yl]-6-(4-morpholinyl)-1,3,5-triazin-2-yl]piperidinyl}-N-methylacetamide in 95% yield: mp (CH₂Cl₂/hexanes) 201-203° C.; ¹H NMR (DMSO-d₆) (rotamers) δ 7.90 (t, J=6.6 Hz, 1H), 7.69 and 7.65 (2t, J_{HF}=53.0 Hz, 1H), 6.95 and 6.94 (2d, J=8.0, 7.9 Hz, 1H), 4.74-4.20 (m, 4H), 3.98 (2s, 3H), 3.80 (m, 4H), 3.69 (m, 4H), 3.22-3.01 (m, 1H), 2.98-2.85 (m, 5H), 1.96-151 (m, 4H); Anal. Calcd. for C₂₄H₂₉ClF₂N₈O₃: C, 52.3; H, 5.3; N, 20.3. Found: C, 52.2; H, 5.4; N, 20.6%.

### Example 36

### Synthesis of 2-chloro-N-{(3S)-1-[4-[2-(difluoromethyl)-4-methoxy-1H-benzimidazol-1-yl]-6-(4-morpholinyl)-1,3,5-triazin-2-yl]piperidinyl}-N-methylacetamide

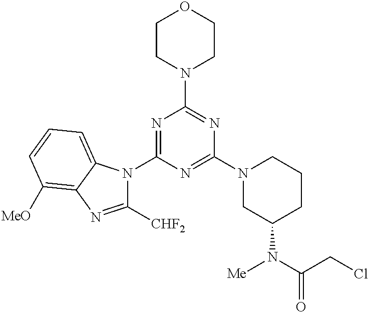

The compound was prepared according to Scheme 1 using Method A.

Similar methylation of tert-butyl (3S)-1-[4-[2-(difluoromethyl)-4-methoxy-1H-benzimidazol-1-yl]-6-(4-morpholinyl)-1,3,5-triazin-2-yl]piperidinylcarbamate (Example 34) with NaH and MeI gave tert-butyl (3S)-1-[4-[2-(difluoromethyl)-4-methoxy-1H-benzimidazol-1-yl]-6-(4-morpholinyl)-1,3,5-triazin-2-yl]piperidinyl(methyl)carbamate in 83% yield: ¹H NMR (DMSO-d₆) (rotamers) δ 7.89 (d, J=8.4 Hz, 1H), 7.69 and 7.66 (2t, J_{HF}=52.9, 52.8 Hz, 1H), 7.43-7.34 (m, 1H), 6.94 (d, J=8.0 Hz, 1H), 4.71-4.56 (m, 2H), 3.97 (s, 3H), 3.80-3.69 (m, 9H), 3.07-2.78 (m, 5H), 1.88-1.79 (m, 3H), 1.50-1.25 (m, 1H), 1.41 (s, 9H); Anal. Calcd. for C₂₇H₃₆F₂N₈O₄: C, 56.4; H, 6.3; N, 19.5. Found: C, 56.5; H, 6.3; N, 19.5%.

Deprotection of the above carbamate with TFA in CH₂Cl₂ gave (3S)-1-[4-[2-(difluoromethyl)-4-methoxy-1H-benzimidazol-1-yl]-6-(4-morpholinyl)-1,3,5-triazin-2-yl]-N-methyl-3-piperidinamine in 100% yield: ¹H NMR (DMSO-d₆) (rotamers) δ 8.42 (br, exchangeable with D₂O, 1H), 7.89 (d, J=8.3 Hz, 1H), 7.72 and 7.69 (2t, J_{HF}=52.5, 53.1 Hz, 1H), 7.42 (t, J=8.2, 1H), 6.96 (d, J=8.0 Hz, 1H), 4.40-4.30, 4.16-4.09 and 3.97-3.92 (m, 2H), 3.98 (s, 3H), 3.82-3.49 (m, 10H), 3.22-3.17 (m, 1H), 2.64 and 2.60 (2s, 3H), 2.11-2.06 (m, 1H), 1.81-1.72 (m, 2H), 1.56-1.55 (m, 1H).

Reaction of the crude amine with chloroacetyl chloride as before gave 2-chloro-N-{(3S)-1-[4-[2-(difluoromethyl)-4-methoxy-1H-benzimidazol-1-yl]-6-(4-morpholinyl)-1,3,5-triazin-2-yl]piperidinyl}-N-methylacetamide in 77% yield: ¹H NMR (DMSO-d₆) (rotamers) δ 7.90 (t, J=6.8 Hz, 1H), 7.69 and 7.65 2 (2t, J_{HF}=53.0, 52.6 Hz, 1H), 7.43-7.33 (m, 1H), 6.95 and 6.94 (2d, J=7.9 Hz, 1H), 4.74-4.49 and 4.33-4.21 (2m, 3H), 4.44 and 4.41 (ABq, J=13.6, 13.0 Hz, 2H), 3.98 and 3.97 (2s, 3H), 3.80 (m, 4H), 3.69 (m, 4H), 3.20-3.05 (m, 1H), 2.98 and 2.96 (2s, 3H), 2.93-2.85 (m, 1H), 1.96-1.52 (m, 4H); Anal. Calcd. for C₂₄H₂₉ClF₂N₈O₃: C, 52.3; H, 5.3; N, 20.3. Found C, 52.3, H, 5.6; N, 20.6%.

### Example 37

### Synthesis of N-[(3R)-1-(chloroacetyl)piperidinyl]-4-[2-(difluoromethyl)-4-methoxy-1H-benzimidazol-1-yl]-N-methyl-6-(4-morpholinyl)-1,3,5-triazin-2-amine

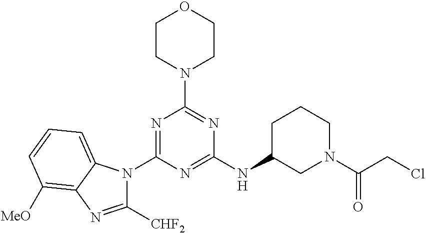

Similarly, reaction of 1-[4-chloro-6-(4-morpholinyl)-1,3,5-triazin-2-yl]-2-(difluoromethyl)-4-methoxy-1H-benzimidazole with tert-butyl (3R)-3-amino-1-piperidinecarboxylate gave tert-butyl (3R)-3-{[4-[2-(difluoromethyl)-4-methoxy-1H-benzimidazol-1-yl]-6-(4-morpholinyl)-1,3,5-triazin-2-yl]amino}-1-piperidinecarboxylate in 88% yield: ¹H NMR (DMSO-d₆) (rotamers) δ 8.11 and 8.00 (2d, J=8.1, 8.2 Hz, 1H), 7.89 and 7.71 (2t, J_{HF}=53.1 Hz, 1H), 7.38 (t, J=8.2 Hz, 1H), 6.95 and 6.94 (2d, J=8.0, 7.8 Hz, 1H), 3.97 (s, 3H), 3.79-3.69 (m, 10H), 3.02-2.80 (m, 2H), 1.97-1.93 (m, 1H), 1.76 (m, 1H), 1.59-1.20 (m, 2H), 1.35 (s, 9H); Anal. Calcd. for C₂₆H₃₄F₂N₈O₄: C, 55.7; H, 6.1; N, 20.0. Found: C, 55.9; H, 6.1; N, 20.1%.

Deprotection of the above carbamate with TFA in CH₂Cl₂ gave 4-[2-(difluoromethyl)-4-methoxy-1H-benzimidazol-1-yl]-6-(4-morpholinyl)-N-[(3R)-piperidinyl]-1,3,5-triazin-2-amine in 98% yield: ¹H NMR (DMSO-d₆) (rotamers) δ 8.10 and 7.93 (2d, J=8.3 Hz, 1H), 7.88 and 7.73 (2t, J_{HF}=53.0 Hz, 1H), 7.84 and 7.91 (2d, J=7.7, 7.9 Hz, 1H), 7.43-7.36 (m, 1H), 6.95 and 6.94 (2d, J=7.9 Hz, 1H), 3.98 and 3.97 (2s, 3H), 3.93-3.91 (m, 1H), 3.79 (m, 4H), 3.69 (m, 4H), 3.18-3.09 (m, 1H), 2.93-2.89 (m, 1H), 2.60-2.52 (m, 2H), 1.96-1.94 and 1.73-1.70 (2m, 2H), 1.56-1.42 (m, 2H).

Reaction of the crude amine with chloroacetyl chloride as before gave N-[(3R)-1-(chloroacetyl)piperidinyl]-4-[2-(difluoromethyl)-4-methoxy-1H-benzimidazol-1-yl]-6-(4-morpholinyl)-1,3,5-triazin-2-amine in 96% yield: mp (CH₂Cl₂/hexanes) 190-192° C.; ¹H NMR (DMSO-d₆) (rotamers) δ 8.11-7.56 (m, 3H), 7.38 (t, J=8.2 Hz, 1H), 6.94 (d, J=8.1 Hz, 1H), 4.59-4.25 (m, 3H), 4.09-3.69 (m, 1H), 3.17-3.03 (m, 1H), 2.88-2.79 and 2.60-2.59 (2m, 1H), 2.0-1.99 (m, 1H), 1.80-1.77 (m, 1H), 1.67-1.41 (m, 2H); Anal. Calcd. for C₂₃H₂₇ClF₂N₈O₃: C, 51.5; H, 5.1; N, 20.9. Found: C, 51.5; H, 5.2; N, 20.6%.

### Example 38

### Synthesis of N-[(3S)-1-(chloroacetyl)piperidinyl]-4-[2-(difluoromethyl)-4-methoxy-1H-benzimidazol-1-yl]-6-(4-morpholinyl)-1,3,5-triazin-2-amine

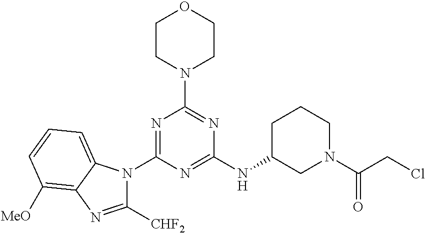

Similarly, reaction of 1-[4-chloro-6-(4-morpholinyl)-1,3,5-triazin-2-yl]-2-(difluoromethyl)-4-methoxy-1H-benzimidazole with tert-butyl (3S)-3-amino-1-piperidinecarboxylate gave tert-butyl (3S)-3-{[4-[2-(difluoromethyl)-4-methoxy-1H-benzimidazol-1-yl]-6-(4-morpholinyl)-1,3,5-triazin-2-yl]amino}-1-piperidinecarboxylate in 90% yield: ¹H NMR (DMSO-d₆) (rotamers) δ 8.12-7.58 (m, 3H), 7.38 and 7.38 (2t, J=8.2 Hz, 1H), 6.95 and 6.94 (2d, J=7.8 Hz, 1H), 3.97 (s, 3H), 3.78-3.69 (m, 10H), 2.99-2.94 and 2.85-2.83 (2m, 2H), 1.97-1.93 and 1.78-1.76 (2m, 2H), 1.58-1.19 (m, 11H).

Deprotection of the carbamate with TFA in CH₂Cl₂ gave 4-[2-(difluoromethyl)-4-methoxy-1H-benzimidazol-1-yl]-6-(4-morpholinyl)-N-[(3S)-piperidinyl]-1,3,5-triazin-2-amine in 100% yield: ¹H NMR (DMSO-d₆) (rotamers) δ 8.78 (m, 2H), 8.11-7.59 (m, 3H), 7.43-7.37 (m, 1H), 6.98-6.94 (m, 1H), 4.20-4.15 (m, 1H), 3.99 and 3.97 (2s, 3H), 3.80 (m, 4H), 3.70 (m, 4H), 3.44-3.22 (m, 2H), 2.90-2.76 (m, 2H), 2.04-1.91 (m, 2H), 1.77-1.53 (m, 2H).

Reaction of the crude amine with chloroacetyl chloride as before gave N-[(3S)-1-(chloroacetyl)piperidinyl]-4-[2-(difluoromethyl)-4-methoxy-1H-benzimidazol-1-yl]-6-(4-morpholinyl)-1,3,5-triazin-2-amine in 83% yield: mp (CH₂Cl₂/hexanes) 191-192° C.; ¹H NMR (DMSO-d₆) (rotamers) δ 8.11-7.56 (m, 3H), 7.38 (t, J=8.2 Hz, 1H), 6.94 (d, J=8.1 Hz, 1H), 4.59-4.25 (m, 3H), 4.09-3.69 (m, 10H), 3.97 (s, 3H), 3.16-3.02, 2.88-2.79 and 2.64-2.58 (3m, 2H), 2.01-1.98 and 1.80-1.77 (2m, 2H), 1.68-1.41 (m, 2H); Anal. Calcd. for C₂₃H₂₇F₂N₈O₃: C, 51.5; H, 5.1; N, 20.9. Found C, 51.4; H, 5.0; N, 20.4%.

### Example 39

### Synthesis of N-[(3R)-1-(chloroacetyl)piperidinyl]-4-[2-(difluoromethyl)-4-methoxy-1H-benzimidazol-1-yl]-N-methyl-6-(4-morpholinyl)-1,3,5-triazin-2-amine

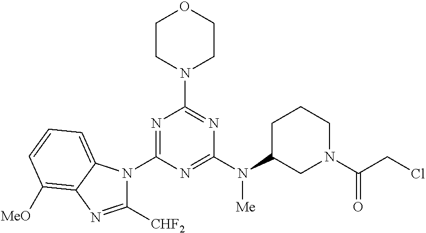

Methylation of tert-butyl (3R)-3-{[4-[2-(difluoromethyl)-4-methoxy-1H-benzimidazol-1-yl]-6-(4-morpholinyl)-1,3,5-triazin-2-yl]amino}-1-piperidinecarboxylate (Example 37) with NaH and MeI in DMF gave tert-butyl (3R)-3-[[4-[2-(difluoromethyl)-4-methoxy-1H-benzimidazol-1-yl]-6-(4-morpholinyl)-1,3,5-triazin-2-yl](methyl)amino]-1-piperidinecarboxylate in 89% yield: mp (CH₂Cl₂/hexanes) 201-203° C.; ¹H NMR (DMSO-d₆) (rotamers) δ 7.97 and 7.89 (2d, J=8.3, 8.1 Hz, 1H), 7.74 and 7.63 (2t, J_{HF}=52.6, 52.9 Hz, 1H), 7.43-7.34 (m, 1H), 6.95 and 6.94 (2d, J=8.1 Hz, 1H), 4.55-4.40 (m, 1H), 3.97 (s, 3H), 3.82-3.70 (m, 9H), 3.11 and 3.07 (2s, 3H), 2.98-2.81 and 2.71-2.66 (2m, 2H), 1.87-1.76 (3H), 1.45-1.24 (m, 1H), 1.40 (s, 9H); Anal. Calcd. for C₂₇H₃₆F₂N₈O₃: C, 56.4; H, 6.4; N, 19.5. Found: C, 56.9: H, 6.3; N, 19.4%.

Deprotection of the carbamate with TFA in CH₂Cl₂ gave 4-[2-(difluoromethyl)-4-methoxy-1H-benzimidazol-1-yl]-N-methyl-6-(4-morpholinyl)-N-[(3R)-piperidinyl]-1,3,5-triazin-2-amine in 93% yield: ¹H NMR (DMSO-d₆) (rotamers) δ 7.97 and 7.93 (2d, J=8.4 Hz, 1H), 7.74 and 7.71 (2t, J_{HF}=52.9, 53.1 Hz, 1H), 7.40 (t, J=8.2 Hz, 1H), 6.95 (d, J=8.1 Hz, 1H), 4.53-4.46 (m, 1H), 3.98 (s, 3H), 3.80 (m, 4H), 3.70-3.69 (m, 4H), 3.08 and 3.05 (2s, 3H), 2.93-2.88 (m, 2H), 2.72-2.63 (m, 1H), 2.43-2.37 (m, 1H), 1.80-1.65 (m, 3H), 1.56-1.47 (m, 1H).

Reaction of the crude amine with chloroacetyl chloride as before gave N-[(3R)-1-(chloroacetyl)piperidinyl]-4-[2-(difluoromethyl)-4-methoxy-1H-benzimidazol-1-yl]-N-methyl-6-(4-morpholinyl)-1,3,5-triazin-2-amine in 97% yield: mp (CH₂Cl₂/hexanes) 194-197° C.; ¹H NMR (DMSO-d₆) (rotamers) δ 8.00 and 7.86 (2d J=8.2, 8.8 Hz, 1H), 7.74 and 7.68 (2t, J_{HF}=51.5, 52.3 Hz, 1H), 7.43-7.35 (m, 1H), 6.95-6.94 (m, 1H), 4.65-4.24 (m, 4H), 3.98 and 3.97 (2s, 3H), 3.81 (m, 4H), 3.70 (m, 4H), 3.14 and 3.10 (2s, 3H), 3.10-3.02, 2.93-2.87, 2.83-2.78 and 2.63-2.53 (4m, 2H), 1.93-1.84 (m, 3H), 1.67-1.39 (m, 1H); Anal. Calcd. for C₂₂H₂₈F₂N₈O₂: C, 52.3, H, 5.31; N, 20.3. Found C, 52.8; H, 5.5, 20.1%.

### Example 40

### Synthesis of N-[(3S)-1-(chloroacetyl)piperidinyl]-4-[2-(difluoromethyl)-4-methoxy-1H-benzimidazol-1-yl]-N-methyl-6-(4-morpholinyl)-1,3,5-triazin-2-amine

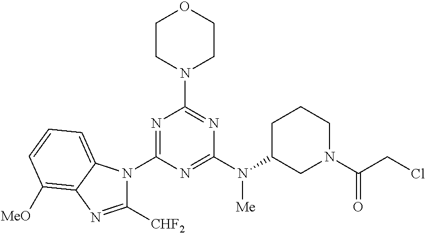

Similarly, methylation of tert-butyl (3S)-3-{[4-[2-(difluoromethyl)-4-methoxy-1H-benzimidazol-1-yl]-6-(4-morpholinyl)-1,3,5-triazin-2-yl]amino}-1-piperidinecarboxylate (Example 38) with NaH and MeI in DMF gave tert-butyl (3S)-3-[[4-[2-(difluoromethyl)-4-methoxy-1H-benzimidazol-1-yl]-6-(4-morpholinyl)-1,3,5-triazin-2-yl](methyl)amino]-1-piperidinecarboxylate: ¹H NMR (DMSO-d₆) (rotamers) δ 7.97 and 7.88 (2d, J=8.3, 8.1 Hz, 1H), 7.74 and 7.63 (2t, J_{HF}=52.5, 52.9 Hz, 1H), 7.43-7.34 (m, 1H), 6.95 and 6.95 (2d, J=8.1 Hz, 1H), 4.57-4.40 (m, 1H), 3.97 (s, 3H), 3.94-3.67 (m, 10H), 3.12 and 3.07 (2s, 3H), 2.98-2.94 (m, 1H), 2.74-2.66 (m, 1H), 1.82-1.76 (m, 3H), 1.40 (s, 9H), 1.45-1.24 (m, 1H); Anal. Calcd. for C₂₇H₃₆F₂N₈O₃: C, 56.4; H, 6.3; N, 19.5. Found C, 56.3, 6.5; N, 19.4%.

Deprotection of the above carbamate with TFA in CH₂Cl₂ gave 4-[2-(difluoromethyl)-4-methoxy-1H-benzimidazol-1-yl]-N-methyl-6-(4-morpholinyl)-N-[(3S)-piperidinyl]-1,3,5-triazin-2-amine in 99% yield: ¹H NMR (DMSO-d₆) (rotamers) δ 7.96 and 7.92 (2d, J=8.4 Hz, 1H), 7.74 and 7.71 (2t, J_{HF}=52.9, 53.1 Hz, 1H), 7.40 (t, J=8.2 Hz, 1H), 6.95 (d, J=8.1 Hz, 1H), 4.61-4.55 (m, 1H), 3.98 (s, 3H), 3.80 (m, 4H), 3.70 (m, 4H), 3.09 and 3.05 (2s, 3H), 2.97-2.95 (s, 2H), 2.78-2.67 and 2.52-2.43 (2m, 2H), 1.79-1.52 (m, 4H).

Reaction of the crude amine with chloroacetyl chloride as before gave N-[(3S)-1-(chloroacetyl)piperidinyl]-4-[2-(difluoromethyl)-4-methoxy-1H-benzimidazol-1-yl]-N-methyl-6-(4-morpholinyl)-1,3,5-triazin-2-amine in 83% yield: ¹H NMR (DMSO-d₆) (rotamers) δ 7.97 and 7.86 (2d, J=8.4, 8.8 Hz, 1H), 7.74 and 7.68 (2t, J_{HF}=50.8, 52.9 Hz, 1H), 7.43-7.35 (m, 1H), 6.96-6.92 (m, 1H), 4.66-4.79 (m, 4H), 3.98 and 3.99 (2s, 3H), 3.81 (m, 4H), 3.70 (m, 4H), 3.40-3.20 and 3.10-3.02 (2m, 1H), 3.14 and 3.10 (2s, 3H), 3.95-2.78 (m, 1H), 2.63-2.45 (m, 1H), 1.96-1.78 (m, 3H), 1.53-1.40 (m, 1H); Anal. Calcd. for C₂₂H₂₉F₂N₈O₂: C, 52.3; H, 5.3; N, 20.3. Found: C, 52.5; H, 5.3; N, 20.3%.

### Example 41

### Synthesis of 2-chloro-N-{1-[4-[2-(difluoromethyl)-4-methoxy-1H-benzimidazol-1-yl]-6-(4-morpholinyl)-1,3,5-triazin-2-yl]-3-azetidinyl}acetamide

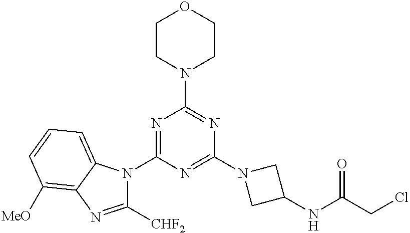

Reaction of 1-[4-chloro-6-(4-morpholinyl)-1,3,5-triazin-2-yl]-2-(difluoromethyl)-4-methoxy-1H-benzimidazole and tert-butyl 3-azetidinylcarbamate gave tert-butyl 1-[4-[2-(difluoromethyl)-4-methoxy-1H-benzimidazol-1-yl]-6-(4-morpholinyl)-1,3,5-triazin-2-yl]-3-azetidinylcarbamate in 90% yield: mp (CH₂Cl₂/hexanes) 217-220° C.; ¹H NMR (DMSO-d₆) δ 7.98 (d, J=8.0 Hz, 1H), 7.73 (t, J_{HF}=53.0 Hz, 1H), 8.24 (t, J=8.2 Hz, 1H), 6.95 (d, J=7.8 Hz, 1H), 4.42 and 4.36-4.32 (2m, 3H), 4.01-3.98 (m, 2H), 3.79-3.77 (m, 4H), 3.68 (m, 4H), 1.40 (s, 9H); Anal. Calcd. for C₂₄H₃₀F₂N₈O₄: C, 54.1; H, 5.7; N, 21.0. Found C, 54.4; H, 5.8; N, 21.2%.

Deprotection of the above carbamate with TFA in CH₂Cl₂ gave 1-[4-[2-(difluoromethyl)-4-methoxy-1H-benzimidazol-1-yl]-6-(4-morpholinyl)-1,3,5-triazin-2-yl]-3-azetidinamine in 100% yield: ¹H NMR (DMSO-d₆) δ 7.99 (d, J=8.4 Hz, 1H), 7.74 (t, J_{HF}=53.1 Hz, 1H), 7.40 (t, J=8.2 Hz, 1H), 6.94 (d, J=7.8 Hz, 1H), 4.35-4.32 and 4.27-4.23 (2m, 2H), 3.97 (s, 3H), 3.87-3.68 (m, 10H), 2.21 (br, exchangeable with D₂O, 2H).

Reaction of the crude amine with chloroacetyl chloride as before gave 2-chloro-N-{1-[4-[2-(difluoromethyl)-4-methoxy-1H-benzimidazol-1-yl]-6-(4-morpholinyl)-1,3,5-triazin-2-yl]-3-azetidinyl}acetamide in 95% yield: mp (CH₂Cl₂/hexanes) 272-275° C.; ¹H NMR (DMSO-d₆) δ 7.99 (d, J=8.0 Hz, 1H), 7.74 (t, J_{HF}=53.0 Hz, 1H), 7.40 (t, J=8.2 Hz, 1H), 6.95 (d, J=7.8 Hz, 1H), 4.67-4.59 (m, 1H), 4.49-4.45 and 4.40-4.36 (2m, 2H), 4.10 (s, 2H), 4.10-3.98 (m, 2H), 3.98 (s, 3H), 3.80-3.78 (m, 4H), 3.68 (m, 4H); Anal. Calcd. for C₂₁H₂₃ClF₂N₈O₃: C, 49.6; H, 4.6: N, 22.0. Found C, 49.8; H, 4.7; N, 21.9%.

### Example 42

### Synthesis of 2-chloro-N-{1-[4-[2-(difluoromethyl)-4-methoxy-1H-benzimidazol-1-yl]-6-(4-morpholinyl)-1,3,5-triazin-2-yl]-3-azetidinyl}-N-methylacetamide

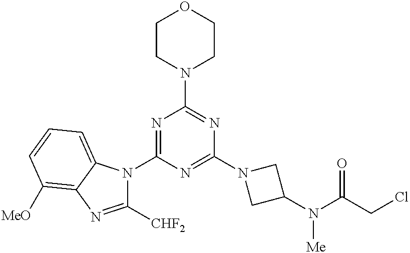

Methylation of tert-butyl 1-[4-[2-(difluoromethyl)-4-methoxy-1H-benzimidazol-1-yl]-6-(4-morpholinyl)-1,3,5-triazin-2-yl]-3-azetidinylcarbamate (Example 41) with NaH and MeI in DMF gave tert-butyl 1-[4-[2-(difluoromethyl)-4-methoxy-1H-benzimidazol-1-yl]-6-(4-morpholinyl)-1,3,5-triazin-2-yl]-3-azetidinyl(methyl)carbamate in 90% yield: mp (CH₂Cl₂/hexanes) 174-177° C.; ¹H NMR (DMSO-d₆) δ 8.00 (d, J=8.0 Hz, 1H), 7.75 (t, J_{HF}=53.0 Hz, 1H), 7.39 (t, J=8.2 Hz, 1H), 6.95 (d, J=7.8 Hz, 1H), 4.88 (br, 1H), 4.40-4.15 (m, 4H), 3.97 (s, 3H), 3.80-3.78 (m, 4H), 3.69 (m, 4H), 2.89 (s, 3H); Anal. Calcd. for C₂₅H₃₂F₂N8O₄: C, 54.9, H, 5.9, H, 20.5. Found: C, 55.2; H, 6.0; N, 20.6%.

Deprotection of the above carbamate with TFA in CH₂Cl₂ gave 1-[4-[2-(difluoromethyl)-4-methoxy-1H-benzimidazol-1-yl]-6-(4-morpholinyl)-1,3,5-triazin-2-yl]-N-methyl-3-azetidinamine in 94% yield: ¹H NMR (DMSO-d₆) δ 7.98 (d, J=8.3 Hz, 1H), 7.74 (t, J_{HF}=53.0 Hz, 1H), 7.41 (t, J=8.2 Hz, 1H), 7.17 (br, exchangeable with D₂O, 1H), 6.96 (d, J=8.0 Hz, 1H), 4.46-4.43 (m, 2H), 4.22-4.06 (m, 3H), 3.98 (s, 3H), 3.82-3.79 (m, 4H), 3.69 (m, 4H), 2.59 (s, 3H).

Reaction of the crude amine with chloroacetyl chloride as before gave 2-chloro-N-{1-[4-[2-(difluoromethyl)-4-methoxy-1H-benzimidazol-1-yl]-6-(4-morpholinyl)-1,3,5-triazin-2-yl]-3-azetidinyl}-N-methylacetamide in 85% yield: mp (CH₂Cl₂/hexanes) 233-236° C.; ¹H NMR (DMSO-d₆) δ 8.00 (d, J=8.3 Hz, 1H), 7.75 (t, J_{HF}=53.0 Hz, 1H), 7.40 (t, J=8.2 Hz, 1H), 6.95 (d, J=8.0 Hz, 1H), 5.26-5.20 and 5.08-5.01 (2m, 1H), 4.48 and 4.45 (2s, 2H), 4.43-4.14 (m, 2H), 3.98 (s, 3H), 3.80-3.78 (m, 4H), 3.69 (m, 4H) 3.09 and 2.99 (2s, 3H); Anal. Calcd. for C₂₂H₂₅ClF₂N₈O₃: C, 50.5; H, 4.8; N, 21.4. Found: C, 51.2, H, 5.0; N, 21.4%.

### Example 43

### Synthesis of N-[1-(chloro acetyl)-3-azetidinyl]-4-[2-(difluoromethyl)-4-methoxy-1H-benzimidazol-1-yl]-6-(4-morpholinyl)-1,3,5-triazin-2-amine

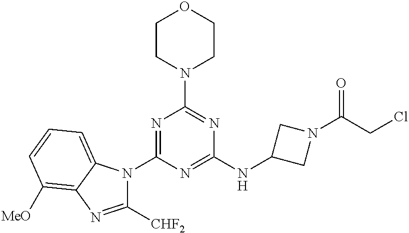

Reaction of 1-[4-chloro-6-(4-morpholinyl)-1,3,5-triazin-2-yl]-2-(difluoromethyl)-4-methoxy-1H-benzimidazole with tert-butyl 3-amino-1-azetidinecarboxylate gave tert-butyl 3-{[4-[2-(difluoromethyl)-4-methoxy-1H-benzimidazol-1-yl]-6-(4-morpholinyl)-1,3,5-triazin-2-yl]amino}-1-azetidinecarboxylate in 86% yield: mp (CH₂Cl₂/hexanes) 201-203° C.; ¹H NMR (DMSO-d₆) (rotamers) δ 8.50 and 8.45 (2d, J=6.3, 6.5 Hz, 1H), 8.01 and 7.93 (2d, J=8.2, 8.3 Hz, 1H), 7.83 and 7.44 (2t, J_{HF}=53.1, 52.9 Hz, 1H), 7.44-7.37 (m, 1H), 6.96 and 6.95 (2d, J=8.1, 8.0 Hz, 1H), 4.73-4.58 (m, 1H), 4.78 (t, J=7.8 Hz, 2H), 3.97 (s, 3H), 3.87-3.82 (m, 2H), 3.78 (m, 4H), 3.69, (m, 4H), 1 40 and 1.39 (2 s, 9H); Anal. Calcd. for C₂₄H₃₀F₂N₈O₄: C, 54.1; H, 5.7; N, 21.0. Found: C, 54.0; 5.8; N, 21.0%.

Deprotection of the above carbamate with TFA in CH₂Cl₂ gave N-(3-azetidinyl)-4-[2-(difluoromethyl)-4-methoxy-1H-benzimidazol-1-yl]-6-(4-morpholinyl)-1,3,5-triazin-2-amine in 100% yield: ¹H NMR (DMSO-d₆) (rotamers) δ 8.58 and 8.54 (2d, J=5.9, 6.6 Hz, 1H), 8.07 and 7.91 (2d, J=8.4, 8.3 Hz, 1H), 7.82 and 7.70 (2t, J_{HF}=51.3 and 52.9 Hz, 1H), 7.44-7.38 (m, 1H), 6.97 and 6.96 (2d, J=7.9, 8.0 Hz, 1H), 4.98-4.88 and 4.88-4.80 (2m, 1H), 4.23-4.16 (m, 2H), 4.06-3.98 (m, 2H), 3.98 (s, 3H), 3.80-3.78 (m, 4H), 3.70 (m, 4H).

Reaction of the crude amine with chloroacetyl chloride as before gave N-[1-(chloroacetyl)-3-azetidinyl]-4-[2-(difluoromethyl)-4-methoxy-1H-benzimidazol-1-yl]-6-(4-morpholinyl)-1,3,5-triazin-2-amine in 95% yield: mp (CH₂Cl₂/hexanes) 219-221° C.; ¹H NMR (DMSO-d₆) (rotamers) δ 8.57 and 8.53 (2d, J=5.9, 6.4 Hz, 1H), 8.09 and 7.95 (2d J=8.3, 7.1 Hz, 1H) 7.82 and 7.71 (2t, J_{HF}=41.6, 52.9 Hz, 1H), 7.45-7.37 (m, 1H), 6.96 and 6.95 (2d, J=7.9, 8.0 Hz, 1H), 4.82-4.70 and 4.72-4.53 (2m, 1H), 4.58-4.52 (m, 1H), 4.28-4.11 (m, 4H), 3.98 (s, 3H), 3.79 (m, 4H), 3.69 (m, 4H); Anal. Calcd. for C₂₁H₂₃ClF₂N₈O₃: C, 49.6; H, 4.6: N, 22.0. Found: C, 49.5; H, 4.5; N, 21.7%.

### Example 44

### Synthesis of N-[1-(chloroacetyl)-3-azetidinyl]-4-[2-(difluoromethyl)-4-methoxy-1H-benzimidazol-1-yl]-N-methyl-6-(4-morpholinyl)-1,3,5-triazin-2-amine

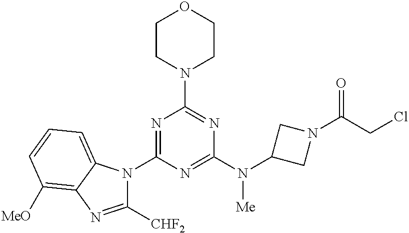

Methylation of N-(3-azetidinyl)-4-[2-(difluoromethyl)-4-methoxy-1H-benzimidazol-1-yl]-6-(4-morpholinyl)-1,3,5-triazin-2-amine (Example 43) with NaH and MeI in DMF gave tert-butyl 3-[[4-[2-(difluoromethyl)-4-methoxy-1H-benzimidazol-1-yl]-6-(4-morpholinyl)-1,3,5-triazin-2-yl](methyl)amino]-1-azetidinecarboxylate in 91% yield: mp (CH₂Cl₂/hexanes) 221-223° C.; ¹H NMR (DMSO-d₆) δ 7.97-7.95 (m, 2H), 7.42 (t, J=8.2 Hz, 1H), 6.95 (d, J=7.9 Hz, 1H), 5.52-5.48 and 5.36-5.34 (2m, 1H), 4.16-4.06 (m, 4H), 3.98 (s, 3H), 3.81 (m, 4H), 3.70-3.69 (m, 4H), 3.23 (s, 3H), 1.41 (s, 9H); Anal. Calcd. for C₂₅H₃₂F₂N₈O₄: C, 54.9; H, 5.9; N, 20.5. Found: C, 55.0, H, 6.0; N, 20.4%.

Deprotection of the above carbamate with TFA in CH₂Cl₂ gave N-(3-azetidinyl)-4-[2-(difluoromethyl)-4-methoxy-1H-benzimidazol-1-yl]-N-methyl-6-(4-morpholinyl)-1,3,5-triazin-2-amine in 96% yield: ¹H NMR (DMSO-d₆) δ 7.95-7.56 (m, 2H), 7.42 (t, J=8.2 Hz, 1H), 6.96 (d, J=8.0 Hz, 1H), 5.60 and 5.38 (2 m, 1H), 4.27-4.22 (m, 2H), 4.17-4.12 (m, 2H), 3.98 (s, 3H), 3.81 (m, 4H), 3.71 (m, 4H), 3.24 (s, 3H).

Reaction of the crude amine with chloroacetyl chloride as before gave N-[1-(chloroacetyl)-3-azetidinyl]-4-[2-(difluoromethyl)-4-methoxy-1H-benzimidazol-1-yl]-N-methyl-6-(4-morpholinyl)-1,3,5-triazin-2-amine in 52% yield: mp (CH₂Cl₂/hexanes) 220-222° C.; ¹H NMR (DMSO-d₆) (rotamers) δ 7.96 and 7.87 (2d, J=8.2, 7.7 Hz, 1H), 7.73 and 7.71 (2t, J_{HF}=52.7, 53.6 Hz, 1H), 7.42 (t, J=8.2 Hz, 1H), 6.96 (d, J=7.8 Hz, 1H), 5.64-5.61 and 5.46-5.38 (2m, 1H), 4.54-4.35 (m, 2H), 4.26-4.12 (m, 2H), 4.21 (s, 2H), 3.97 (s, 3H), 3.80 (m, 4H), 3.69 (m, 4H), 3.24 (s, 3H); Anal. Calcd. for C₂₂H₂₅ClF₂N₈O₃: C, 50.5; H, 4.8; N, 21.4. Found C, 50.3, H, 4.8; N, 21.1%.

### Example 45

### Synthesis of 1-[4-{[1-(chloroacetyl)-4-piperidinyl]oxy}-6-(4-morpholinyl)-1,3,5-triazin-2-yl]-2-(difluoromethyl)-4-methoxy-1H-benzimidazole

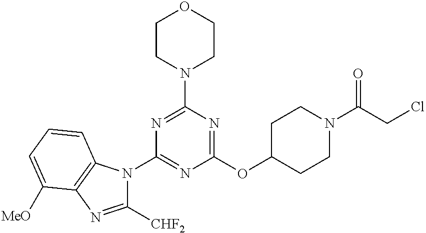

The compound was prepared according to Scheme 1 using Method A.

A mixture of 0.47 g (2 mmol) of 2,4-dichloro-6-(4-morpholinyl)-1,3,5-triazine, 0.40 g of tert-butyl 4-hydroxy-1-piperidinecarboxylate (2 mmol) and a small excess of NaH in 20 mL of THF was stirred at room temperature overnight, before being quenched with water and worked up in EtOAc. Chromatography on silica, eluting with CH₂Cl₂/EtOAc (9:1) gave 0.65 g (81% yield) of tert-butyl 4-(4-chloro-6-morpholino-1,3,5-triazin-2-yloxy)piperidine-1-carboxylate, as a white solid: mp (i-Pr₂O) 150-152° C.; ¹H NMR (CDCl₃) δ 5.18 (tt, J=7.7, 3.8 Hz, 1H), 3.87 (m, 2H), 3.83 (m, 2H), 3.78-3.71 (m, 6H), 3.30 (ddd, J=13.5, 8.3, 3.8 Hz, 2H), 1.95 (m, 2H), 1.78 (m, 2H), 1.46 (s, 9H); Anal. Calcd. for C₁₇H₂₆ClN₅O₄: C, 51.06; H, 6.55; N, 17.51. Found: C, 51.21; H, 6.28; N, 17.4%.

A mixture of 175 mg (0.44 mmol) of the above chloro compound, 100 mg (0.505 mmol) of 2-(difluoromethyl)-4-methoxy-1H-benzimidazole and 0.28 g (2 mmol) of powdered K₂CO₃ in 10 mL DMSO was stirred at room temperature for 3 days, and diluted with water. The resulting precipitate was collected, washed with water, and dried. Chromatography on silica eluting with CH₂Cl₂/EtOAc (4:1) gave 200 mg (81% yield) of tert-butyl 4-(4-(2-(difluoromethyl)-4-methoxy-1H-benzimidazol-1-yl)-6-morpholino-1,3,5-triazin-2-yloxy)piperidine-1-carboxylate: mp (CH₂Cl₂-MeOH) 191-193° C.; ¹H NMR (CDCl₃) δ 7.96 (dd, J=8.4, 0.5 Hz, 1H), 7.49 (t, J_{HF}=53.5 Hz, 1H), 7.38 (t, J=8.3 Hz, 1H), 6.85 (d, J=7.8 Hz, 1H), 5.25 (m, 1H), 4.06 (s, 3H), 3.96-3.78 (m, 10H), 3.28 (m, 2H), 2.50 (m, 2H), 1.85 (m, 2H), 1.48 (s, 9H); Anal. Calcd. for C₂₆H₃₃F₂N₇O₅: C, 55.61; H, 5.92; N, 17.46. Found: C, 55.77; H, 5.92; N, 17.40%.

Treatment of 112 mg (0.2 mmol) of the above carbamate with TFA (5 mL) in CH₂Cl₂ (10 mL), followed by quenching with aq. NH₃ gave 2-(difluoromethyl)-4-methoxy-1-(4-morpholinyl)-6-(4-piperidinyloxy)-1,3,5-triazin-2-yl]-1H-benzimidazole: ¹H NMR (DMSO-d₆) δ 7.96 (dd, J=8.4, 0.5 Hz, 1H), 7.71 (t, J=52.8 Hz, 1H), 7.44 (t, J=8.3 Hz, 1H), 6.98 (d, J=7.7 Hz, 1H), 5.11 (m, 1H), 3.98 (s, 3H), 3.83 (m, 4H), 3.71 (m, 4H), 3.00 (m, 2H), 2.62 (m, 2H), 2.00 (m, 2H), 1.58 (m, 2H).

Treatment of the crude amine with chloroacetyl chloride as before, followed by chromatography on silica eluting with CH₂Cl₂/EtOAc (4:1) gave 61 mg (57% yield) of 1-[4-{[1-(chloroacetyl)-4-piperidinyl]oxy}-6-(4-morpholinyl)-1,3,5-triazin-2-yl]-2-(difluoromethyl)-4-methoxy-1H-benzimidazole: mp (aq. MeOH) 170-172° C.; ¹H NMR (CDCl₃) δ 7.95 (d, J=8.3 Hz, 1H), 7.47 (t, J_{HF}=53.5 Hz, 1H), 7.38 (t, J=8.3 Hz, 1H), 6.85 (d, J=8.0 Hz, 1H), 5.37 (tt, J=7.1, 3.6 Hz, 1H), 4.11 (d, J=1.6 Hz, 2H), 4.06 (s, 3H), 3.94 (m, 5H), 3.80 (m, 5H), 3.65 (m, 1H), 3.53 (m, 1H), 2.18-1.92 (m, 4H); Anal. Calcd. for C₂₃H₂₆ClF₂N₇O₄: C, 51.35; H, 4.87; N, 18.23. Found: C, 51.62; H, 4.88; N, 17.99%.

### Example 46

### Synthesis of 3-{[1-[4-{[1-(chloroacetyl)-4-piperidinyl]oxy}-6-(4-morpholinyl)-1,3,5-triazin-2-yl]-2-(difluoromethyl)-1H-benzimidazol-4-yl]oxy}-N,N-dimethyl-1-propanamine

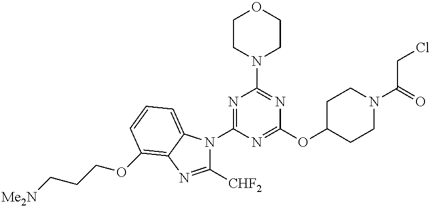

The compound was prepared according to Scheme 1 using Method A.

A mixture of 7.2 g (46.7 mmol) of 2-amino-3-nitrophenol, 6.6 g (70 mmol) 3-chloro-1-propanol, and 19.3 g (0.14 mol) dry powdered K₂CO₃ in 50 mL acetone was heated and stirred under reflux for 20 hrs. The solvent was removed under vacuum and the residue was diluted with water, extracted into ethyl acetate, and dried (Na₂SO₄). Removal of the solvent and chromatography of the residue on silica eluting with CH₂Cl₂/EtOAc (4:1) gave 10.0 g (100% yield) of 2-(2-amino-3-nitrophenoxy)ethanol as a yellower solid: mp (CH₂Cl₂/hexanes) 72-74° C.; ¹H NMR (CDCl₃) δ 7.71 (dd, J=8.9, 1.2 Hz, 1H), 6.92 (d, J=7.7 Hz, 1H), 6.60 (dd, J=8.9, 7.8 Hz, 1H), 6.42 (s, exchangeable with D₂O, 2H), 4.20 (t, J=6.1 Hz, 2H), 3.89 (q, J=5.7 Hz, 2H), 2.12 (pentet, J=6.1 Hz, 2H), 1.48 (t, J=5.0 Hz, exchangeable with D₂O, 1H); Anal. Calcd. for C₉H₁₂N₂O₄: C, 50.9; H, 5.7; N, 13.3. Found: C, 51.0; H, 5.7; N, 13.3%.

A solution of 4.3 g (20.26 mmol) of the above alcohol in 50 mL MeOH was hydrogenated over 10% Pd on C and then filtered into a solution of 5 mL conc. HCl in MeOH. After removal of the solvents the residue was dissolved in a mixture of 60 mL 4 M HCl and 5 mL of difluoroacetic acid, and the resulting mixture was heated under reflux for 6 hrs. After cooling to 20° C., the mixture was diluted with 100 mL water, neutralized with aq. NH₃, extracted into EtOAc, and dried (Na₂SO₄). Evaporation of the solvent gave crude 2-{[2-(difluoromethyl)-1H-benzimidazol-4-yl]oxy}ethanol which was combined with 6.1 g (40 mmol) TBDMSCl in 15 mL pyridine. The mixture was stirred at 20° C. for 3 hrs before being poured into water and extracted with EtOAc. After drying (Na₂SO₄) and evaporation of the solvent, the residue was purified by chromatography on silica eluting with CH₂Cl₂/EtOAc (9:1) to give 5.79 g (84% yield) of 4-(2-{[tert-butyl(dimethyl)silyl]oxy}ethoxy)-2-(difluoromethyl)-1H-benzimidazole: mp (CH₂Cl₂/hexanes) 133-135° C.; ¹H NMR (DMSO-d₆) (tautomers) δ 13.40 and 13.23 (s, exchangeable with D₂O, 1H), 7.36-7.05 (m, 3H), 6.86 and 6.75 (2d, J=7.8 Hz and 7.4 Hz, 1H), 4.27 and 4.22 (2 t, J=6.3 Hz, 2H), 3.87 and 3.81 (2t, J=6.1 Hz, 1H), 2.05-1.95 (m, 2H), 0.85 and 0.84 (2s, 9H), 0.02 and 0.01 (2s, 6H).

A mixture of 942 mg (2.64 mmol) of the above benzimidazole, 148 mg (0.37 mmol) of tert-butyl 4-{[4-chloro-6-(4-morpholinyl)-1,3,5-triazin-2-yl]oxy}-1-piperidinecarboxylate (Example 45), and 1.09 g (7.92 mmol) K₂CO₃ in dry DMSO (2 mL) was heated at 130° C. for 2 hrs, before being cooled and diluted with water. The resulting precipitate was filtered, washed with water and dried. Recrystallization from CH₂Cl₂/hexanes gave 1.47 g (85% yield) of tert-butyl 4-{[4-[4-(3-{[tert-butyl(dimethyl)silyl]oxy}propoxy)-2-(difluoromethyl)-1H-benzimidazol-1-yl]-6-(4-morpholinyl)-1,3,5-triazin-2-yl]oxy}-1-piperidinecarboxylate as a white solid: ¹H NMR (CDCl₃) δ 7.95 (d, J=8.1 Hz, exchangeable with D₂O, 1H), 7.48 (t, J_{HF}=53.5 Hz, 1H), 7.35 (t, J=8.3 Hz, 1H), 6.89 (d, J=8.0 Hz, 1H), 5.29-5.23 (m, 1H), 4.37 (t, J=6.6 Hz, 2H), 3.93-3.91 (m, 4H), 3.86 (d, J=5.9 Hz, 4H), 3.81-3.76 (m, 4H), 3.31-3.25 (m, 2H), 2.15 (q, J=6.3 Hz, 2H), 2.07-2.03 (m, 2H), 1.88-1.80 (m, 2H), 1.48 (s, 9H), 0.89 (s, 9H) 0.05 (s, 6H).

To a suspension of the above silyl ether (1.20 g, 1.67 mmol) in CH₃CN (15 mL) at 0° C. was added BF₃.OEt₂ (3.5 mL). The reaction was stirred for 15 min and diluted with water. The resulting precipitate was collected by filtration, washed with water, and dried to give 3-({2-(difluoromethyl)-1-[4-(4-morpholinyl)-6-(4-piperidinyloxy)-1,3,5-triazin-2-yl]-1H-benzimidazol-4-yl}oxy)-1-propanol as a white solid: ¹H NMR (DMSO-d₆) δ 8.29 (br s, 1H, exchangeable with D₂O), 7.95 (d, J=8.3 Hz, 1H), 7.72 (t, J_{HF}=52.8 Hz, 1H), 7.42 (t, J=8.2 Hz, 1H), 6.99 (d, J=8.0 Hz, 1H), 5.33-5.28 (m, 1H), 4.56 (t, J=5.1 Hz, exchangeable with D₂O, 1H), 4.29 (t, J=6.4 Hz, 2H), 3.85 (m, 4H), 3.74-3.71 (m, 4H), 3.65-3.61 (m, 2H), 3.28 (m, 2H), 3.17-3.11 (m, 2H), 2.21-2.16 (m, 2H), 2.01-1.89 (m, 4H).

To a mixture of the above amino-alcohol (885 mg, 1.75 mmol) and K₂CO₃ (483 mg, 3.50 mmol) in THF (15 mL) and H₂O (5 mL) was added Boc₂O (458 mg, 2.1 mmol). The reaction mixture was stirred at 20° C. for 3 hrs, and then diluted with water. The resulting precipitate was collected by filtration, washed with water, and dried, to give 853 mg (80% yield) of tert-butyl 4-{[4-[2-(difluoromethyl)-4-(3-hydroxypropoxy)-1H-benzimidazol-1-yl]-6-(4-morpholinyl)-1,3,5-triazin-2-yl]oxy}-1-piperidinecarboxylate as a white solid: ¹H NMR (DMSO-d₆) δ 7.95 (d, J=8.37 Hz, 1H), 7.71 (t, J_{HF}=52.8 Hz, 1H), 7.42 (t, J=8.3 Hz, 1H), 6.98 (d, J=8.0 Hz, 1H), 4.56 (t, J=5.1 Hz, 1H), 4.29 (t, J=6.4 Hz, 2H), 3.84 (m, 4H), 3.73-3.60 (m, 6H), 3.64-3.60 (m, 2H), 3.24-3.18 (m, 2H), 2.04-1.92 (m, 4H), 1.69-1.61 (m, 2H), 1.42 (s, 9H).

Methanesulphonyl chloride (4.2 mmol) was added to a solution of the above alcohol (850 mg, 1.4 mmol) and Et₃N (0.4 mL, 2.8 mmol) in THF (10 mL) at 0° C., and the resulting mixture was stirred at 0° C. for 2 hrs. A 40% aq. solution of dimethylamine (2 mL) was then added and the mixture was stirred at 20° C. for 20 hrs. The solvents were removed under vacuum and the residue was diluted with water to give a precipitate, which was collected by filtration, washed with water, and dried to give 834 mg (94% yield) of tert-butyl 4-{[4-{2-(difluoromethyl)-4-[3-(dimethylamino)propoxy]-1H-benzimidazol-1-yl}-6-(4-morpholinyl)-1,3,5-triazin-2-yl]oxy}-1-piperidinecarboxylate as a white solid: ¹H NMR (DMSO-d₆) δ 7.95 (d, J=8.1 Hz, 1H), 7.72 (t, J_{HF}=52.8 Hz, 1H), 7.42 (t, J=8.3 Hz, 1H), 6.97 (d, J=7.9 Hz, 1H), 5.26-5.21 (m, 1H), 4.26 (t, J=6.5 Hz, 2H), 3.84 (m, 4H), 3.74-3.70 (m, 6H), 2.26-3.18 (m, 4H), 2.23 (s, 6H), 2.04-1.94 (m, 4H), 1.69-1.57 (m, 2H), 1.42 (s, 9H).

Deprotection of the above carbamate with TFA in CH₂Cl₂ in similar manner as before gave N-[3-({2-(difluoromethyl)-1-[4-(4-morpholinyl)-6-(4-piperidinyloxy)-1,3,5-triazin-2-yl]-1H-benzimidazol-4-yl}oxy)propyl]-N,N-dimethylamine as a white solid in 97% yield: ¹H NMR (DMSO-d₆) δ 7.95 (d, J=8.1 Hz, 1H), 7.71 (t, J=52.8 Hz, 1H), 7.41 (t, J=8.2 Hz, 1H), 7.37 (br s, exchangeable with D₂O, 1H), 6.97 (d, J=8.0 Hz, 1H), 5.14-5.07 (m, 1H), 4.25 (t, J=6.5 Hz, 2H), 3.83 (m, 4H), 3.83-3.70 (m, 4H), 3.01-2.96 (m, 2H), 2.62-2.56 (m, 2H), 2.41 (t, J=7.1 Hz, 2H), 2.16 (s, 6H), 2.02-1.92 (m, 4H), 1.61-1.52 (m, 2H).

Reaction of the above amine with chloroacetyl chloride and K₂CO₃ in CH₂Cl₂, in a similar manner as before gave 3-{[1-[4-{[1-(chloroacetyl)-4-piperidinyl]oxy}-6-(4-morpholinyl)-1,3,5-triazin-2-yl]-2-(difluoromethyl)-1H-benzimidazol-4-yl]oxy}-N,N-dimethyl-1-propanamine hydrochloride as a white solid in 49% yield: mp (CH₂Cl₂/MeOH/hexanes/EtOAc) 194-197° C.; ¹H NMR (DMSO-d₆) δ 7.99 (d, J=8.3 Hz, 1H), 7.73 (t, J_{HF}=52.7 Hz, 1H), 7.45 (t, J=8.2 Hz, 1H), 7.01 (d, J=8.0 Hz, 1H), 5.35-5.29 (m, 1H), 4.42 (s, 2H), 4.33 (t, J=6.1 Hz, 2H), 3.85 (m, 4H), 3.73-3.71 (m, 4H), 3.45-3.34 (m, 2H), 3.28 (m, 2H), 2.82 (s, 6H), 2.28-2.21 (m, 2H), 2.19-1.19 (m, 2H), 1.86-1.67 (m, 2H); Anal. Calcd. for C₂₇H₃₆O₂F₂N₈O₄.H₂O: C, 48.9; H, 5.7; Cl; 10.7; N, 16.9. Found: C, 49.2; H, 5.7; Cl, 10.8; N, 16.9%.

### Example 47

### Synthesis of 3-{[1-[4-({1-[(chloromethyl)sulfonyl]-4-piperidinyl}oxy)-6-(4-morpholinyl)-1,3,5-triazin-2-yl]-2-(difluoromethyl)-1H-benzimidazol-4-yl]oxy}-N,N-dimethyl-1-propanamine

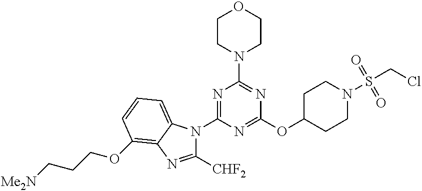

The compound was prepared according to Scheme 1 using Method A.

Reaction of N-[3-({2-(difluoromethyl)-1-[4-(4-morpholinyl)-6-(4-piperidinyloxy)-1,3,5-triazin-2-yl]-1H-benzimidazol-4-yl}oxy)propyl]-N,N-dimethylamine (Example 46) with chloromethanesulphonyl chloride gave 3-{[1-[4-({1-[(chloromethyl)sulfonyl]-4-piperidinyl}oxy)-6-(4-morpholinyl)-1,3,5-triazin-2-yl]-2-(difluoromethyl)-1H-benzimidazol-4-yl]oxy}-N,N-dimethyl-1-propanamine in 16% yield: ¹H NMR (DMSO-d₆) δ 7.95 (d, J=8.4 Hz, 1H), 7.72 (t, J_{HF}=52.8 Hz, 1H), 7.42 (t, J=8.3 Hz, 1H), 6.98 (d, J=8.0 Hz, 1H), 4.25 (t, J=6.5 Hz, 2H), 3.84 (m, 4H), 3.73-3.70 (m, 4H), 3.63-3.58 (m, 4H), 3.63-3.58 (m, 2H), 3.41-3.34 (m, 2H), 2.41 (t, J=7.1 Hz, 2H), 2.16 (s, 6H), 2.12-2.07 (m, 2H), 1.99-1.92 (m, 2H), 1.89-1.80 (m, 2H); Anal. Calcd. for C₂₆H₃₅ClF₂N₈O₅S 0.5H₂O: C, 47.7; H, 5.55; N, 17.1. Found: C, 47.8; H, 5.5; N, 17.1%.

### Example 48

### Synthesis of 1-{1-[(chloromethyl)sulfonyl]-4-piperidinyl}-6-[2-(difluoromethyl)-4-methoxy-1H-benzimidazol-1-yl]-4-(4-morpholinyl)-1H-pyrazolo[3,4-d]pyrimidine

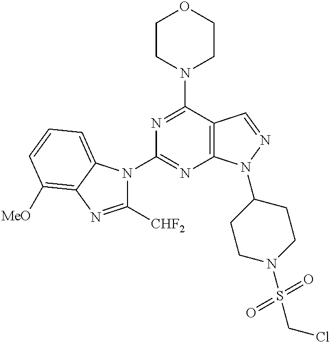

The compound was prepared according to Scheme 1 using Method A.

A stirred mixture of 0.44 g (2.2 mmol) of 2-(difluoromethyl)-4-methoxy-1H-benzimidazole, 0.47 g (1.1 mmol) of tert-butyl 4-[6-chloro-4-(4-morpholinyl)-1H-pyrazolo[3,4-d]pyrimidin-1-yl]-1-piperidinecarboxylate (WO 2008/115974), and 0.61 g (4.4 mmol) of powdered K₂CO₃ in 10 mL of DMSO was heated at 160° C. for 20 hrs. After cooling, the mixture was diluted with water, and the precipitate was collected by filtration, washed with water, and dried. Chromatography on silica eluting with CH₂Cl₂/EtOAc (17:3) gave 0.20 g (31% yield) of tert-butyl 4-[6-[2-(difluoromethyl)-4-methoxy-1H-benzimidazol-1-yl]-4-(4-morpholinyl)-1H-pyrazolo[3,4-d]pyrimidin-1-yl]-1-piperidinecarboxylate: mp (MeOH) 245-247° C.; ¹H NMR (CDCl₃) δ 7.99 (s, 1H), 7.84 (dd, J=8.4, 0.6 Hz, 1H), 7.47 (t, J_{HF}=53.6 Hz, 1H), 7.37 (t, J=8.2 Hz, 1H), 6.83 (d, J=7.8 Hz, 1H), 4.87 (tt, J=11.4, 4.1 Hz, 1H), 4.32 (m, 2H), 4.07 (s, 3H), 4.06 (m, 4H), 3.91 (m, 4H), 2.99 (m, 2H), 2.23 (dq, J=12.3, 4.5 Hz, 2H), 2.02 (m, 2H), 1.49 (s, 9H); Anal. Calcd. for C₂₈H₃₄F₂N₈O₄: C, 57.5; H, 5.9; N, 19.2. Found: C, 57.2; H, 6.0; N, 19.0%.

Treatment of the above carbamate with TFA in CH₂Cl₂ as before gave 6-[2-(difluoromethyl)-4-methoxy-1H-benzimidazol-1-yl]-4-(4-morpholinyl)-1-(4-piperidinyl)-1H-pyrazolo[3,4-d]pyrimidine in 97% yield: ¹H NMR (CDCl₃) δ 8.00 (s, 1H), 7.86 (dd, J=8.4, 0.6 Hz, 1H), 7.51 (t, J_{HF}=53.6 Hz, 1H), 7.38 (t, J=8.2 Hz, 1H), 6.83 (d, J=7.7 Hz, 1H), 4.83 (tt, J=11.6, 4.1 Hz, 1H), 4.07 (s, 3H), 4.06 (m, 4H), 3.91 (m, 4H), 3.31 (m, 2H), 2.87 (dt, J=12.7, 2.5 Hz, 2H), 2.21 (dq, J=12.3, 4.2 Hz, 2H), 2.04 (m, 2H).

A mixture of 85 mg (175 mmol) of the above amine and 73 mg (525 mmol) of powdered K₂CO₃ in CH₂Cl₂ was cooled to 0° C. and 78 mg (525 mmol) of chloromethanesulfonyl chloride was added. The mixture was allowed to warm to room temperature, and after 3 hrs it was diluted with water and washed with aq. NH₃. The organic layer was dried, and removed under vacuum, and the residue was recrystallized from MeOH to give 76 mg (73% yield) of 1-{1-[(chloromethyl)sulfonyl]-4-piperidinyl}-6-[2-(difluoromethyl)-4-methoxy-1H-benzimidazol-1-yl]-4-(4-morpholinyl)-1H-pyrazolo[3,4-d]pyrimidine: mp 250-253° C.; ¹H NMR (CDCl₃) δ 8.01 (s, 1H), 7.82 (dd, J=8.4, 0.6 Hz, 1H), 7.45 (t, J_{HF}=53.6 Hz, 1H), 7.39 (t, J=8.2 Hz, 1H), 6.83 (d, J=7.8 Hz, 1H), 4.90 (tt, J=11.1, 4.1 Hz, 1H), 4.57 (s, 2H), 4.15 (m, 1H), 4.12 (m, 1H), 4.07 (s, 3H), 4.06 (m, 4H), 3.92 (m, 4H), 3.32 (dt J=12.5, 2.6 Hz, 2H), 2.42 (dq, J=12.4, 4.3 Hz, 2H), 2.15 (m, 2H); Anal. Calcd. for C₂₄H₂₇ClF₂N₈O₄S: C, 48.3; H, 4.6; N, 18.8. Found: C, 48.1; H, 4.6; N, 18.6%.

### Example 49

### Biological Activity Determination

### A. Reversible Inhibition of Isolated Enzyme

Compounds were evaluated for their ability to inhibit Class I PI 3-kinase enzymes p110δ/p85, p110α/p85, and p110β/p85. Reaction mixtures comprising 0.1 μg of a recombinant enzyme, 10 μg of L-α-phosphatidylinositol, and 2× Lipid Kinase Buffer (40 mM Tris-HCl, pH 7.4, 200 mM NaCl, 1 mM EDTA), which contains either DMSO only as a control or the test compound in DMSO (the final DMSO concentration is 1%), were activated by the addition of an ATP mix (5 mM MgCl₂, 100 μM ATP, and 0.1 μL [γ³³P]ATP). Reactions were incubated at room temperature for 1 hr, and then stopped by the addition of 1M HCl. The lipids were then extracted using a two step procedure. Firstly, 200 μL of chloroform/methanol (1:1) was added, the biphasic reactions mixed and centrifuged briefly, and the inorganic phase was removed and discarded. Following this, 80 μL of methanol:HCl (1:1) was added and the same procedure followed. The organic phase (70 μL) was then transferred to a clean 1.6 mL tube and the reactions were dried using a Speedvac, with no heating, for 30 min. The reactions were spotted onto TLC plates (Merck Ltd) and developed for 1 hr in propanol-1:2 M acetic acid (13:7). The TLC plates were then dried at room temperature and quantified using a phosphorimager (StormImager, Amersham). Nine compound concentrations were used for each test compound to determine its IC₅₀ value. Each experiment was performed twice and the average IC₅₀ value is used herein. The results are summarised in Table 2.

B. Irreversible and Selective Inhibition of p110α Isoform

The compounds were evaluated for their ability to irreversibly inhibit the p110α isoform of PI3K in both isolated enzyme and cell culture assays.

a. Isolated Enzyme Assay:

Recombinant PI3K (p110/p85) was first subjected to immunoprecipitation. Protein A-Sepharose beads were washed twice with a lysis buffer (20 mM Tris-HCl, 138 mM NaCl, 2.7 mM KCl, 1 mM MgCl₂, 1 mM CaCl₂, 5% (v/v) glycerol, 1% (v/v) Nonidet P-40 (NP40), 5 mM EDTA, 20 μM leupeptin, 18 μM pepstatin, 1 mM AEBSF, 4 μg/mL aprotinin, 2 mM Na₃VO₄, 20 mM NaF, 1 mM DTT (pH7.4)) and then pre-incubated with polyclonal antibodies specific to the N—SH2 domain of p85 alpha (Shepherd et al., J. Biol. Chem. 1997, 272, 19000) for 30 min at 4° C. Recombinant PI3K (p110/p85) was then added for further incubation overnight at 4° C. The following day, immune complexes were washed twice with the lysis buffer, resuspended in the same buffer, and aliquoted. After incubation with the test compounds (1 μM, 30 min at room temperature), immune complexes were then either 1) directly tested for Lipid kinase activity or 2) washed three times with the lysis buffer and then tested for Lipid kinase activity (same protocol as for reversible inhibition of isolated enzyme). Wortmannin (100 nM) and LY 294002 (5 μM) were used as the positive and negative controls, respectively.

b. Cellular Assay:

Fully differentiated 3T3L1 adipocytes were starved overnight without serum. The following day, cells were incubated for 60 min with wortmannin (100 nM) as the positive control, LY 294002 (5 μM) as the negative control, the test compounds (1 μM), or DMSO.

To test for irreversibility, cells were then either: 1) rinsed 3 times with warm medium and incubated without an inhibitor for 30 min; or 2) maintained in the same medium containing an inhibitor.

Cells were subsequently stimulated or not with insulin (100 nM, 10 min). Finally, cells were washed twice with ice-cold Phosphate Buffer Saline (PBS) (140 mM NaCl, 3 mM KCl, 6 mM Na₂HPO₄, 1 mM KH₂PO₄ (pH 7.4)) and solubilized with a lysis buffer (20 mM Tris-HCl, 138 mM NaCl, 2.7 mM KCl, 1 mM MgCl₂, 1 mM CaCl₂, 5% (v/v) glycerol, 1% (v/v) Nonidet P-40 (NP40), 5 mM EDTA, 20 μM leupeptin, 18 μM pepstatin, 1 mM AEBSF, 4 μg/mL aprotinin, 2 mM Na₃VO₄, 20 mM NaF, 1 mM DTT (pH7.4)). Lysates were kept on ice for 20 min and insoluble material was removed by centrifugation at 14,000 g for 10 min. Protein concentration was determined by colorimetric assay (BCA, Pierce).

Proteins were separated by SDS-PAGE and transferred to polyvinylidene difluoride (PVDF) membranes (Pall Corporation, USA). The membranes were incubated for 1 hr in a blocking buffer (20 mM Tris (pH 7.4), 137 mM NaCl, 0.5% (v/v) Tween 20) containing 3% (w/v) BSA (ICP Bio, Auckland, New Zealand) or non-fat milk, and then incubated overnight in the blocking buffer containing specific antibodies (phosphoSer473 PKB and total PKB) Immunoreactive proteins were detected using horseradish peroxidase-linked secondary antibodies (Dako) and enhanced chemiluminescence (ECL) according to the manufacturer's instructions (GE Healthcare, Inc.). Signals were analyzed and quantified using Fuji LAS-3000 phosphorimager and Fuji Image Gauge software.

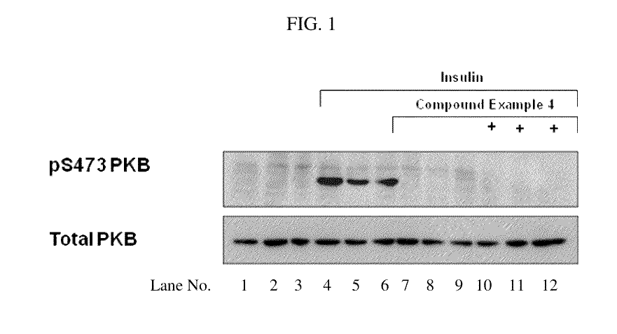

As an example, the results for Compound Example 4 are shown in FIG. 1. Overnight-starved 3T3L1 adipocytes cells were incubated for 60 min with compound of Example 4 at 1 μM (lanes 7-12). The cells in lanes 10-12 were then rinsed 3 times with warm cell culture medium and incubated without the compound for 30 min, whereas compound of Example 4 was left continuously on the other drug exposed cells (lanes 7-9). Cells were then stimulated (lanes 4-12) or not (lanes 1-3) with insulin (100 nM, 10 min) and then lysed. Whole-cell lysates were then analysed by Western blotting using either an antibody that specifically recognised residue 473 of PKB only when it is phosphorylated (phosphoSer⁴⁷³ PKB) or with an antibody that recognizes all forms of PKB (Total PKB). The results demonstrate that insulin stimulates the phosphorylation of PKB in these cells (lanes 4-6) and that this is attenuated by compound 4 (lanes 7-12), even when unbound compound 4 had been washed away prior to exposure of the cells to insulin (lanes 10-12). This indicates that compound 4 has irreversibly bound PI 3-kinase in the cells.

### C. Cellular Growth Inhibition

The compounds were evaluated against two early passage human cell lines NZB5 and NZOV9 (Marshall et al., Oncol. Res. 2004, 14, 297). The cells were grown in ITS medium (α-modified minimal essential medium supplemented insulin, transferrin, selenite, and 5% fetal bovine serum) and grown on 96-well tissue culture plates under an atmosphere of 5% O₂, 5% CO₂, and 90% N₂. Individual wells contained 500-1,000 cells (depending on the growth rate) in a volume of 150 μL. Compounds were added at 10-fold concentration steps to a maximum of 20 μM and plates were incubated for five days, with ³H-thymidine being added over the last 6 hrs. Cells were harvested and incorporated radioactivity measured. Duplicate samples were analyzed for each compound dose with multiple control samples. Data were fitted by a least-squares method to an exponential of the form y=y₀+ae^{-bx}, where y is the radioactivity (corrected for background and normalized to 100% of the control), x is the radiation dose, and y₀, a, and b are variables, and the IC₅₀ value defined as the compound concentration reducing ³H-thymidine levels by 50%. The results are summarised in Table 2.

| | | | | | |
|---|---|---|---|---|---|
| 8 | A | | | | |
| 9 | A | A | A | | |
| 10 | A | B | A | | |
| 11 | A | C | A | B | A |
| 12 | A | | A | | |
| 13 | C | C | B | B | B |
| 14 | A | B | A | A | A |
| 15 | B | B | B | B | B |
| 16 | A | B | A | A | A |
| 17 | A | B | A | A | A |
| 18 | A | B | A | B | B |
| 19 | A | A | A | B | A |
| 20 | B | C | A | B | A |
| 21 | A | B | A | A | A |
| 22 | A | B | B | B | A |
| 23 | A | B | B | B | A |
| 24 | B | B | B | B | A |
| 25 | A | A | A | B | A |
| 26 | A | B | A | B | A |
| 27 | A | B | A | B | A |
| 28 | A | B | A | B | A |
| 29 | A | B | A | B | A |
| 30 | A | A | A | B | A |
| 31 | A | B | A | B | A |
| 32 | A | A | A | B | A |
| 33 | A | B | A | B | A |
| 34 | A | A | A | B | A |
| 35 | A | A | A | B | A |
| 36 | A | A | A | C | A |
| 37 | A | B | A | B | A |
| 38 | A | B | A | B | A |
| 39 | A | A | A | B | A |
| 40 | A | A | A | B | A |
| 41 | A | B | A | B | A |
| 42 | A | B | A | B | A |
| 43 | A | A | A | B | A |
| 44 | A | B | A | B | B |
| 45 | A | B | A | B | A |
| 46 | B | C | A | C | B |
| 47 | | | | B | B |
| 48 | A | C | A | | |

*A. <0.1 μM; B. 0.1-1.0 μM; C. >1.0 μM

It is noted that whereas the compounds show both reversible and irreversible inhibition of the p110α isoform of PI3K, only reversible activity is exhibited against the p110β and p110δ isoforms.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A compound of Formula Ia, Ib, Ic, or Id: 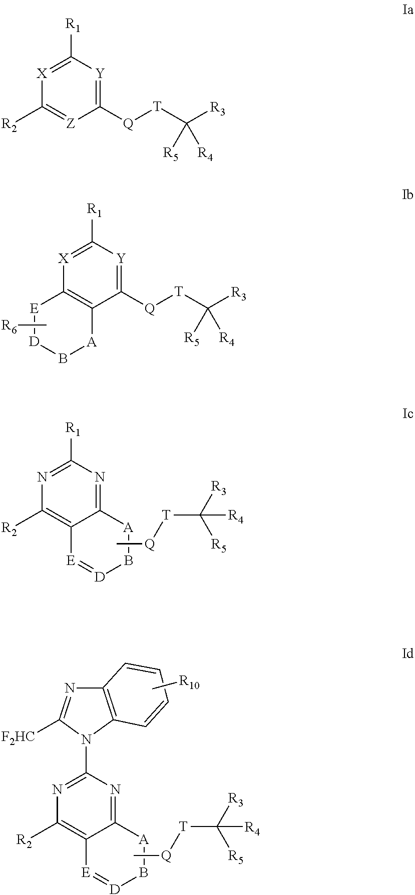
or an enantiomer, a mixture of enantiomers, or a mixture of two or more diastereomers thereof; or a pharmaceutically acceptable salt, solvate, hydrate, or prodrug thereof; wherein:
each R₁ is independently C₆₋₁₄ aryl, heteroaryl, or heterocyclyl;
each R₂ is independently C₆₋₁₄ aryl, heteroaryl, or heterocyclyl;
each R₃ and R₄ is independently hydrogen, lower alkyl, C₂₋₆ alkenyl, C₂₋₆ alkynyl, or R₅;
each R₅ is independently halogen or —OSO₂R₇;
R₆ is C₃₋₇ cycloalkyl, C₆₋₁₄ aryl, heteroaryl, or heterocyclyl;
R₇ is lower alkyl, C₂₋₆ alkenyl, C₂₋₆ alkynyl, C₃₋₇ cycloalkyl, C₆₋₁₄ aryl, heteroaryl, or heterocyclyl;
R₁₀ is (a) hydrogen, amino, or hydroxyl; or (b) lower alkyl, lower alkylamino, di(lower alkyl)amino, lower alkoxy, or carboxamido;
each Q is independently absent or a linker group;
each T is independently —CO—, —CS—, or —SO₂—;
X, Y, and Z are each independently a nitrogen atom or CR₈, with the proviso that at least two of X, Y, and Z are nitrogen atoms; wherein R₈ is hydrogen or lower alkyl; and
each A, B, D, and E is independently (i) a direct bond; (ii) a nitrogen, oxygen, or sulfur atom; or (iii) CR₉, where R₉ is hydrogen, halogen, or lower alkyl; wherein the bonds between A, B, D, and E may be saturated or unsaturated; with the proviso that no more than one of A, B, D, and E are a direct bond;
wherein each alkyl, alkenyl, alkynyl, alkoxy, alkylamino, dialkylamino, carboxamido, cycloalkyl, aryl, heteroaryl, and heterocyclyl is optionally substituted with one or more groups, each independently selected from (a) cyano, halo, and nitro; (b) C₁₋₆ alkyl, C₂₋₆ alkenyl, alkynyl, C₃₋₇ cycloalkyl, C₆₋₁₄ aryl, C₇₋₁₅ aralkyl, heteroaryl, and heterocyclyl, each optionally substituted with one or more, in one embodiment, one, two, three, or four, substituents Q¹; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiment, one, two, three, or four, substituents Q¹; or (iii) R^{b} and R^{c} together with the N atom to which they are attached form heterocyclyl, optionally substituted with one or more, in one embodiment, one, two, three, or four, substituents Q¹;
wherein each Q¹ is independently selected from the group consisting of (a) cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. The compound of claim 1, having the structure of Formula IV: 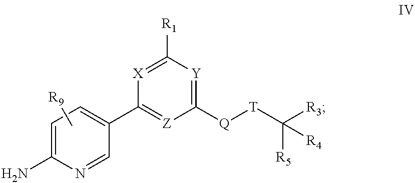
wherein R₉ is hydrogen, lower alkyl, fluoromethyl, difluoromethyl, trifluoromethyl, cyano, amino, lower alkylamino, hydroxyl, lower alkoxy, or halogen.

3. The compound of claim 1, having the structure of Formula V: 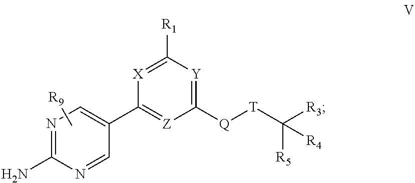
wherein R₉ is hydrogen, lower alkyl, fluoromethyl, difluoromethyl, trifluoromethyl, cyano, amino, lower alkylamino, hydroxyl, lower alkoxy, or halogen.

4. The compound of claim 1, having the structure of Formula VII:

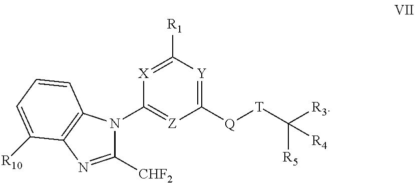

5. The compound of claim 1, having the structure of Formula VIII: 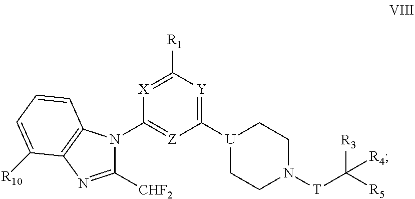
wherein U is CH or N.

6. The compound of claim 1, having the structure of Formula IX: 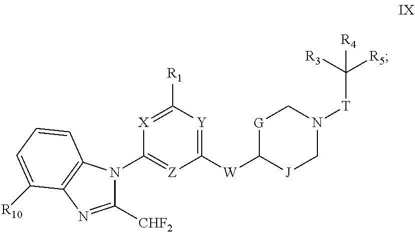
wherein:
G and J are each independently a direct bond or —CH₂—; and
W is a direct bond; or oxygen, sulfur, or NR₁₁; where R₁₁ is hydrogen, or substituted or unsubstituted lower alkyl.

7. The compound of claim 6, wherein W is NR₁₁.

8. The compound of claim 1, having the structure of Formula X: 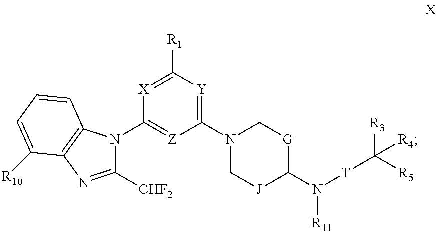
wherein:
R₁₁ is hydrogen, or substituted or unsubstituted lower alkyl; and
G and J are each independently a direct bond or —CH₂—.

9. The compound of claim 6, wherein G is a direct bond.

10. The compound of claim 6, wherein G is —CH₂—.

11. The compound of claim 6, wherein J is a direct bond.

12. The compound of claim 6, wherein J is —CH₂—.

13. The compound of claim 1, having the structure of Formula XI: 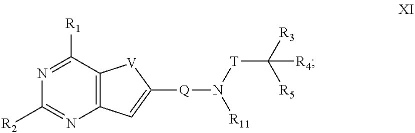
wherein:
R₁₁ is hydrogen, or substituted or unsubstituted lower alkyl; and
V is oxygen or sulfur.

14. The compound of claim 6, wherein R₁₁ is hydrogen or methyl.

15. The compound of claim 1, having the structure of Formula XII:

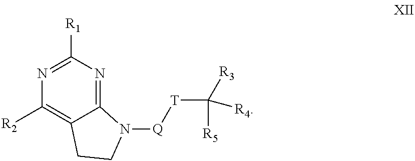

16. The compound of claim 1, having the structure of Formula XIII: 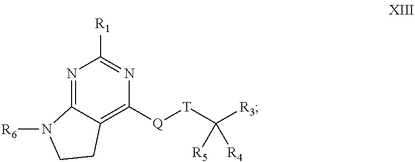
wherein R₆ is substituted or unsubstituted aryl or heteroaryl.

17. The compound of claim 1, having the structure of Formula XIII:

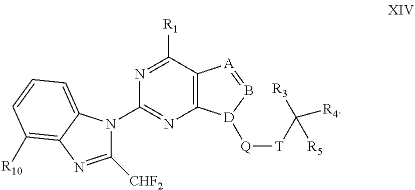

18. The compound of claim 17, wherein A is CH.

19. The compound of claim 17, wherein B is N.

20. The compound of claim 17, wherein D is N.

21. The compound of claim 1, wherein R₂ is substituted or unsubstituted aryl or substituted heteroaryl.

22. The compound of claim 21, wherein R₂ is hydroxyphenyl, hydroxymethylphenyl, aminopyridyl, aminopyrimidyl, indazolyl, difluoromethyl-1H-benzimidazolyl, difluoromethyl-hydroxy-1H-benzimidazolyl, difluoromethyl-methoxy-1H-benzimidazolyl, difluoromethyl-ethoxy-1H-benzimidazolyl, or difluoromethyl-((N,N-dimethylamino)propoxy)-1H-benzimidazolyl.

23. The compound of claim 22, wherein R₂ is 3-hydroxyphenyl, 3-hydroxymethylphenyl, 2-amino-pyrid-5-yl, 2-amino-pyrimid-5-yl, indazol-4-yl, 2-difluoromethyl-1H-benzimidazolyl, 2-difluoromethyl-4-hydroxy-1H-benzimidazolyl, 2-difluoromethyl-4-methoxy-1H-benzimidazolyl, 2-difluoromethyl-4-ethoxy-1H-benzimidazolyl, or 2-difluoromethyl-4-(3-(N,N-dimethylamino)propoxy)-1H-benzimidazolyl;

24. The compound of claim 1, wherein R₁₀ is hydrogen, hydroxy, or substituted or unsubstituted lower alkoxy.

25. The compound of claim 24, wherein R₁₀ is hydrogen, hydroxy, methoxy, ethoxy, or 3-(N,N-dimethylamino)propoxy.

26. The compound of claim 1, wherein R₁ is substituted or unsubstituted morpholino or pyridyl.

27. The compound of claim 26, wherein R₁ is substituted or unsubstituted 4-morpholino or 4-pyridyl.

28. The compound of claim 26, wherein R₁ is 4-morpholino or 4-pyridyl.

29. The compound of claim 1, wherein R₃ is hydrogen, chloro, or methyl.

30. The compound of claim 1, wherein R₄ is hydrogen, chloro, or methyl.

31. The compound of claim 1, wherein R₅ is chloro, bromo, or —OSO₂-methyl.

32. The compound of claim 1, wherein Q is substituted or unsubstituted heterocyclylene.

33. The compound of claim 32, wherein Q is a divalent linker selected from the group consisting of azetidinyleneamino, azetidinylene(methylamino), piperidyleneoxy, piperazinylene, piperidylene, piperidyleneamino, piperidylene(methylamino), pyrrolidinyleneamino, pyrrolidinylene(methylamino), and piperidylenethio.

34. The compound of claim 33, wherein Q is a divalent linker selected from the group consisting of azetidinylene-4-amino, azetidinylene-4-methylamino, piperidylene-4-oxy, 1,4-piperazinylene, 1,4-piperidylene, piperidylene-3-amino, (R)-piperidylene-3-amino, (S)-piperidylene-3-amino, piperidylene-3-methylamino, (R)-piperidylene-3-methylamino, (S)-piperidylene-3-methylamino, piperidylene-4-amino, piperidylene-4-methylamino, pyrrolidinylene-3-amino, (R)-pyrrolidinylene-3-amino, (S)-pyrrolidinylene-3-amino, pyrrolidinylene-3-methylamino, (R)-pyrrolidinylene-3-methylamino, (S)-pyrrolidinylene-3-methylamino, and piperidylene-4-thio.

35. The compound of claim 1, wherein T is —CO— or —SO₂—.

36. The compound of claim 1, wherein X is N or CH.

37. The compound of claim 1, wherein Y is N or CH.

38. The compound of claim 1, wherein Z is N or CH.

39. The compound of claim 1 selected from the group consisting of:
3-[4-[4-(chloroacetyl)-1-piperazinyl]-6-(4-morpholinyl)-1,3,5-triazin-2-yl]phenol;
1-[4-[4-(chloroacetyl)-1-piperazinyl]-6-(4-morpholinyl)-1,3,5-triazin-2-yl]-2-(difluoromethyl)-4-ethoxy-1H-benzimidazole;
1-[4-[4-(chloroacetyl)-1-piperazinyl]-6-(4-morpholinyl)-1,3,5-triazin-2-yl]-2-(difluoromethyl)-1H-benzimidazole;
1-[4-[4-(chloroacetyl)-1-piperazinyl]-6-(4-morpholinyl)-1,3,5-triazin-2-yl]-2-(difluoromethyl)-4-methoxy-1H-benzimidazole;
1-[4-[4-(chloroacetyl)-1-piperazinyl]-6-(4-morpholinyl)-1,3,5-triazin-2-yl]-2-(difluoromethyl)-1H-benzimidazol-4-ol;
1-[4-[4-(chloroacetyl)-1-piperazinyl]-6-(4-pyridinyl)-1,3,5-triazin-2-yl]-2-(difluoromethyl)-4-methoxy-1H-benzimidazole;
1-[4-[4-(dichloroacetyl)-1-piperazinyl]-6-(4-morpholinyl)-1,3,5-triazin-2-yl]-2-(difluoromethyl)-4-methoxy-1H-benzimidazole;
2-(difluoromethyl)-1-{4-(4-morpholinyl)-6-[4-(trichloroacetyl)-1-piperazinyl]-1,3,5-triazin-2-yl}-1H-benzimidazol-4-yl methyl ether;
2-{-4-[4-[2-(difluoromethyl)-4-methoxy-1H-benzimidazol-1-yl]-6-(4-morpholinyl)-1,3,5-triazin-2-yl]-1-piperazinyl}-2-oxoethyl methanesulfonate;
1-[4-[4-(2-chloropropanoyl)-1-piperazinyl]-6-(4-morpholinyl)-1,3,5-triazin-2-yl]-2-(difluoromethyl)-4-methoxy-1H-benzimidazole;
1-[4-{4-[(chloromethyl)sulfonyl]-1-piperazinyl}-6-(4-morpholinyl)-1,3,5-triazin-2-yl]-2-(difluoromethyl)-4-methoxy-1H-benzimidazole;
1-[4-{4-[(bromomethyl)sulfonyl]-1-piperazinyl}-6-(4-morpholinyl)-1,3,5-triazin-2-yl]-2-(difluoromethyl)-4-methoxy-1H-benzimidazole;
N-(3-{[1-[4-{4-[(chloromethyl)sulfonyl]-1-piperazinyl}-6-(4-morpholinyl)-1,3,5-triazin-2-yl]-2-(difluoromethyl)-1H-benzimidazol-4-yl]oxy}propyl)-N,N-dimethylamine;
1-[4-{4-[(chloromethyl)sulfonyl]-1-piperazinyl}-6-(4-morpholinyl)-2-pyrimidinyl]-2-(difluoromethyl)-4-methoxy-1H-benzimidazole;
1-[6-{4-[(chloromethyl)sulfonyl]-1-piperazinyl}-2-(4-morpholinyl)-4-pyrimidinyl]-2-(difluoromethyl)-4-methoxy-1H-benzimidazole;
1-[2-{4-[(chloromethyl)sulfonyl]-1-piperazinyl}-6-(4-morpholinyl)-4-pyrimidinyl]-2-(difluoromethyl)-4-methoxy-1H-benzimidazole;
1-[4-[1-(chloroacetyl)-4-piperidinyl]-6-(4-morpholinyl)-1,3,5-triazin-2-yl]-2-(difluoromethyl)-4-methoxy-1H-benzimidazole;
N-[1-(chloroacetyl)-4-piperidinyl]-4-[2-(difluoromethyl)-4-methoxy-1H-benzimidazol-1-yl]-6-(4-morpholinyl)-1,3,5-triazin-2-amine;
N-[1-(chloroacetyl)-4-piperidinyl]-4-[2-(difluoromethyl)-4-methoxy-1H-benzimidazol-1-yl]-N-methyl-6-(4-morpholinyl)-1,3,5-triazin-2-amine;
N-[1-(chloroacetyl)-4-piperidinyl]-4-[2-(difluoromethyl)-4-[3-(dimethylamino)propoxy]-1H-benzimidazol-1-yl]-N-methyl-6-(4-morpholinyl)-1,3,5-triazin-2-amine;
N-{1-[(chloromethyl)sulfonyl]-4-piperidinyl}-4-[2-(difluoromethyl)-4-methoxy-1H-benzimidazol-1-yl]-N-methyl-6-(4-morpholinyl)-1,3,5-triazin-2-amine;
2-chloro-N-{1-[4-[2-(difluoromethyl)-4-methoxy-1H-benzimidazol-1-yl]-6-(4-morpholinyl)-1,3,5-triazin-2-yl]-4-piperidinyl}acetamide;
2-chloro-N-{1-[4-[2-(difluoromethyl)-4-methoxy-1H-benzimidazol-1-yl]-6-(4-morpholinyl)-1,3,5-triazin-2-yl]-4-piperidinyl}-N-methylacetamide;
2-chloro-N-{1-[4-{2-(difluoromethyl)-4-[3-(dimethylamino)propoxy]-1H-benzimidazol-1-yl}-6-(4-morpholinyl)-1,3,5-triazin-2-yl]-4-piperidinyl}-N-methylacetamide;
2-chloro-N-{(3R)-1-[4-[2-(difluoromethyl)-4-methoxy-1H-benzimidazol-1-yl]-6-(4-morpholinyl)-1,3,5-triazin-2-yl]pyrrolidinyl}acetamide;
2-chloro-N-{(3S)-1-[4-[2-(difluoromethyl)-4-methoxy-1H-benzimidazol-1-yl]-6-(4-morpholinyl)-1,3,5-triazin-2-yl]pyrrolidinyl}acetamide;
2-chloro-N-{(3R)-1-[4-[2-(difluoromethyl)-4-methoxy-1H-benzimidazol-1-yl]-6-(4-morpholinyl)-1,3,5-triazin-2-yl]pyrrolidinyl}-N-methylacetamide;
2-chloro-N-{(3S)-1-[4-[2-(difluoromethyl)-4-methoxy-1H-benzimidazol-1-yl]-6-(4-morpholinyl)-1,3,5-triazin-2-yl]pyrrolidinyl}-N-methylacetamide;
N-[(3R)-1-(chloroacetyl)pyrrolidinyl]-4-[2-(difluoromethyl)-4-methoxy-1H-benzimidazol-1-yl]-6-(4-morpholinyl)-1,3,5-triazin-2-amine;
N-[(3S)-1-(chloroacetyl)pyrrolidinyl]-4-[2-(difluoromethyl)-4-methoxy-1H-benzimidazol-1-yl]-6-(4-morpholinyl)-1,3,5-triazin-2-amine;
N-[(3R)-1-(chloroacetyl)pyrrolidinyl]-4-[2-(difluoromethyl)-4-methoxy-1H-benzimidazol-1-yl]-N-methyl-6-(4-morpholinyl)-1,3,5-triazin-2-amine;
N-[(3S)-1-(chloroacetyl)pyrrolidinyl]-4-[2-(difluoromethyl)-4-methoxy-1H-benzimidazol-1-yl]-N-methyl-6-(4-morpholinyl)-1,3,5-triazin-2-amine;
2-chloro-N-{(3R)-1-[4-[2-(difluoromethyl)-4-methoxy-1H-benzimidazol-1-yl]-6-(4-morpholinyl)-1,3,5-triazin-2-yl]piperidinyl}acetamide;
2-chloro-N-{(3S)-1-[4-[2-(difluoromethyl)-4-methoxy-1H-benzimidazol-1-yl]-6-(4-morpholinyl)-1,3,5-triazin-2-yl]piperidinyl}acetamide;
2-chloro-N-{(3R)-1-[4-[2-(difluoromethyl)-4-methoxy-1H-benzimidazol-1-yl]-6-(4-morpholinyl)-1,3,5-triazin-2-yl]piperidinyl}-N-methylacetamide;
2-chloro-N-{(3S)-1-[4-[2-(difluoromethyl)-4-methoxy-1H-benzimidazol-1-yl]-6-(4-morpholinyl)-1,3,5-triazin-2-yl]piperidinyl}-N-methylacetamide;
N-[(3R)-1-(chloroacetyl)piperidinyl]-4-[2-(difluoromethyl)-4-methoxy-1H-benzimidazol-1-yl]-N-methyl-6-(4-morpholinyl)-1,3,5-triazin-2-amine;
N-[(3S)-1-(chloroacetyl)piperidinyl]-4-[2-(difluoromethyl)-4-methoxy-1H-benzimidazol-1-yl]-6-(4-morpholinyl)-1,3,5-triazin-2-amine;
N-[(3R)-1-(chloroacetyl)piperidinyl]-4-[2-(difluoromethyl)-4-methoxy-1H-benzimidazol-1-yl]-N-methyl-6-(4-morpholinyl)-1,3,5-triazin-2-amine;
N-[(3S)-1-(chloroacetyl)piperidinyl]-4-[2-(difluoromethyl)-4-methoxy-1H-benzimidazol-1-yl]-N-methyl-6-(4-morpholinyl)-1,3,5-triazin-2-amine;
2-chloro-N-{1-[4-[2-(difluoromethyl)-4-methoxy-1H-benzimidazol-1-yl]-6-(4-morpholinyl)-1,3,5-triazin-2-yl]-3-azetidinyl}acetamide;
2-chloro-N-{1-[4-[2-(difluoromethyl)-4-methoxy-1H-benzimidazol-1-yl]-6-(4-morpholinyl)-1,3,5-triazin-2-yl]-3-azetidinyl}-N-methyl acetamide;
N-[1-(chloroacetyl)-3-azetidinyl]-4-[2-(difluoromethyl)-4-methoxy-1H-benzimidazol-1-yl]-6-(4-morpholinyl)-1,3,5-triazin-2-amine;
N-[1-(chloroacetyl)-3-azetidinyl]-4-[2-(difluoromethyl)-4-methoxy-1H-benzimidazol-1-yl]-N-methyl-6-(4-morpholinyl)-1,3,5-triazin-2-amine;
1-[4-{[1-(chloroacetyl)-4-piperidinyl]oxy}-6-(4-morpholinyl)-1,3,5-triazin-2-yl]-2-(difluoromethyl)-4-methoxy-1H-benzimidazole;
3-{[1-[4-{[1-(chloroacetyl)-4-piperidinyl]oxy}-6-(4-morpholinyl)-1,3,5-triazin-2-yl]-2-(difluoromethyl)-1H-benzimidazol-4-yl]oxy}-N,N-dimethyl-1-propanamine;
3-{[1-[4-{[1-[(chloromethyl)sulfonyl]-4-piperidinyl]oxy}-6-(4-morpholinyl)-1,3,5-triazin-2-yl]-2-(difluoromethyl)-1H-benzimidazol-4-yl]oxy}-N,N-dimethyl-1-propanamine; and
1-{1-[(chloromethyl)sulfonyl]-4-piperidinyl}-6-[2-(difluoromethyl)-4-methoxy-1H-benzimidazol-1-yl]-4-(4-morpholinyl)-1H-pyrazolo[3,4-d]pyrimidine;
and enantiomers, mixtures of enantiomers, or mixtures of two or more diastereomers thereof;
and pharmaceutically acceptable salts, solvates, hydrates, and prodrugs thereof.

40. The compound of claim 1 selected from the group consisting of:
1-[4-[4-(chloroacetyl)-1-piperazinyl]-6-(4-pyridinyl)-1,3,5-triazin-2-yl]-2-(difluoromethyl)-1H-benzimidazole;
1-[4-[4-(chloroacetyl)-1-piperazinyl]-6-(4-pyridinyl)-1,3,5-triazin-2-yl]-2-(difluoromethyl)-4-ethoxy-1H-benzimidazole;
1-[4-[4-(chloroacetyl)-1-piperazinyl]-6-(4-pyridinyl)-1,3,5-triazin-2-yl]-2-(difluoromethyl)-1H-benzimidazol-4-ol;
3-[4-[4-(chloroacetyl)-1-piperazinyl]-6-(4-pyridinyl)-1,3,5-triazin-2-yl]phenol;
[3-[4-[4-(chloroacetyl)-1-piperazinyl]-6-(4-morpholinyl)-1,3,5-triazin-2-yl]phenyl]methanol;
[3-[4-[4-(chloroacetyl)-1-piperazinyl]-6-(4-pyridinyl)-1,3,5-triazin-2-yl]phenyl]methanol;
5-[4-[4-(chloroacetyl)-1-piperazinyl]-6-(4-morpholinyl)-1,3,5-triazin-2-yl]-2-pyridinamine;
5-[4-[4-(chloroacetyl)-1-piperazinyl]-6-(4-pyridinyl)-1,3,5-triazin-2-yl]-2-pyridinamine;
5-[4-[4-(chloroacetyl)-1-piperazinyl]-6-(4-morpholinyl)-1,3,5-triazin-2-yl]-2-pyrimidinamine;
5-[4-[4-(chloroacetyl)-1-piperazinyl]-6-(4-pyridinyl)-1,3,5-triazin-2-yl]-2-pyrimidinamine;
4-[4-[4-(chloroacetyl)-1-piperazinyl]-6-(4-morpholinyl)-1,3,5-triazin-2-yl]-1H-indazole;
4-[4-[4-(chloroacetyl)-1-piperazinyl]-6-(4-pyridinyl)-1,3,5-triazin-2-yl]-1H-indazole;
1-[4-[1-(chloroacetyl)-4-piperidinyl]-6-(4-morpholinyl)-1,3,5-triazin-2-yl]-2-(difluoromethyl)-1H-benzimidazole;
1-[4-[1-(chloroacetyl)-4-piperidinyl]-6-(4-morpholinyl)-1,3,5-triazin-2-yl]-2-(difluoromethyl)-1H-benzimidazol-4-ol;
1-[4-[1-(chloroacetyl)-4-piperidinyl]-6-(4-morpholinyl)-1,3,5-triazin-2-yl]-2-(difluoromethyl)-4-ethoxy-1H-benzimidazole;
N-[1-(chloroacetyl)-4-piperidinyl]-4-[2-(difluoromethyl)-1H-benzimidazol-1-yl]-6-(4-morpholinyl)-1,3,5-triazin-2-amine;
1-[4-{[1-(chloroacetyl)-4-piperidinyl]amino}-6-(4-morpholinyl)-1,3,5-triazin-2-yl]-2-(difluoromethyl)-1H-benzimidazol-4-ol;
N-[1-(chloroacetyl)-4-piperidinyl]-4-[2-(difluoromethyl)-4-ethoxy-1H-benzimidazol-1-yl]-6-(4-morpholinyl)-1,3,5-triazin-2-amine;
1-[4-{[1-(chloroacetyl)-4-piperidinyl]oxy}-6-(4-morpholinyl)-1,3,5-triazin-2-yl]-2-(difluoromethyl)-1H-benzimidazole;
1-[4-{[1-(chloroacetyl)-4-piperidinyl]oxy}-6-(4-morpholinyl)-1,3,5-triazin-2-yl]-2-(difluoromethyl)-1H-benzimidazol-4-ol;
1-[4-{[1-(chloroacetyl)-4-piperidinyl]oxy}-6-(4-morpholinyl)-1,3,5-triazin-2-yl]-2-(difluoromethyl)-4-ethoxy-1H-benzimidazole;
1-[4-{[1-(chloroacetyl)-4-piperidinyl]sulfanyl}-6-(4-morpholinyl)-1,3,5-triazin-2-yl]-2-(difluoromethyl)-1H-benzimidazole;
1-[4-{[1-(chloroacetyl)-4-piperidinyl]sulfanyl}-6-(4-morpholinyl)-1,3,5-triazin-2-yl]-2-(difluoromethyl)-1H-benzimidazol-4-ol;
1-[4-{[1-(chloroacetyl)-4-piperidinyl]sulfanyl}-6-(4-morpholinyl)-1,3,5-triazin-2-yl]-2-(difluoromethyl)-4-methoxy-1H-benzimidazole;
1-[4-{[1-(chloroacetyl)-4-piperidinyl]sulfanyl}-6-(4-morpholinyl)-1,3,5-triazin-2-yl]-2-(difluoromethyl)-4-ethoxy-1H-benzimidazole;
2-chloro-N-{1-[4-[2-(difluoromethyl)-1H-benzimidazol-1-yl]-6-(4-morpholinyl)-1,3,5-triazin-2-yl]-4-piperidinyl}acetamide;
2-chloro-N-{1-[4-[2-(difluoromethyl)-4-hydroxy-1H-benzimidazol-1-yl]-6-(4-morpholinyl)-1,3,5-triazin-2-yl]-4-piperidinyl}acetamide; and
2-chloro-N-{1-[4-[2-(difluoromethyl)-4-ethoxy-1H-benzimidazol-1-yl]-6-(4-morpholinyl)-1,3,5-triazin-2-yl]-4-piperidinyl}acetamide;
and enantiomers, mixtures of enantiomers, or mixtures of two or more diastereomers thereof;
and pharmaceutically acceptable salts, solvates, hydrates, and prodrugs thereof.

41. A pharmaceutical composition comprising a compound of claim 1, or an enantiomer, a mixture of enantiomers, or a mixture of two or more diastereomers thereof; or a pharmaceutically acceptable salt, solvate, hydrate, or prodrug thereof; in combination with one or more pharmaceutically acceptable carriers.

42. The pharmaceutical composition of claim 41, further comprising a second therapeutic agent.

43. The pharmaceutical composition of claim 41, wherein the composition is formulated for single dose administration.

44. The pharmaceutical composition of claim 43, wherein the composition is formulated as oral, parenteral, or intravenous dosage form.

45. The pharmaceutical composition of claim 44, wherein the oral dosage form is a tablet or capsule.

46. A method for the treatment, prevention, or amelioration of one or more symptoms of a PI3K-mediated disorder, disease, or condition in a subject, which comprises administering to the subject the compound of claim 1.

47. A method for cancer treatment, which comprises administering to a subject the compound of claim 1.

48. The method of claim 46, wherein the compound is administered in combination with a second therapeutic agent.

49. A method for modulating PI3K enzymatic activity, comprising contacting a PI3K enzyme with the compound of claim 1.
